SEQUENCE LISTING (1115 sequences)

SEQ ID NO: 1 - protein, 7 aa, synthetic construct
NNSTRGG

SEQ ID NO: 2 - protein, 7 aa, synthetic construct
GNSARNI

SEQ ID NO: 3 - protein, 7 aa, synthetic construct
GNSVRDF

SEQ ID NO: 4 - protein, 15 aa, synthetic construct
GGGGGGGGGGGGGGG

SEQ ID NO: 5 - protein, 15 aa, synthetic construct
GGGGSGGGGSGGGGS

SEQ ID NO: 6 - protein, 7 aa, synthetic construct
ANAAKNY

SEQ ID NO: 7 - protein, 7 aa, synthetic construct
FNGIKEM

SEQ ID NO: 8 - protein, 7 aa, synthetic construct
NNHTREV

SEQ ID NO: 9 - protein, 7 aa, synthetic construct
TNKIRDP

SEQ ID NO: 10 - protein, 7 aa, synthetic construct
ANALKNQ

SEQ ID NO: 11 - protein, 7 aa, synthetic construct
FNKDKED

SEQ ID NO: 12 - protein, 7 aa, synthetic construct
NNMIRAV

SEQ ID NO: 13 - protein, 7 aa, synthetic construct
TNNTRQN

SEQ ID NO: 14 - protein, 7 aa, synthetic construct
ANATRLQ

SEQ ID NO: 15 - protein, 7 aa, synthetic construct
FNSTRPD

SEQ ID NO: 16 - protein, 7 aa, synthetic construct
NNNTKNI

SEQ ID NO: 17 - protein, 7 aa, synthetic construct
TNNTRVG

SEQ ID NO: 18 - protein, 7 aa, synthetic construct
ANATRYQ

SEQ ID NO: 19 - protein, 7 aa, synthetic construct
GNATKAG

SEQ ID NO: 20 - protein, 7 aa, synthetic construct
NNNTRNH

SEQ ID NO: 21 - protein, 7 aa, synthetic construct
TNNVKSG

SEQ ID NO: 22 - protein, 7 aa, synthetic construct
ANEYKAH

SEQ ID NO: 23 - protein, 7 aa, synthetic construct
GNDVKDI

SEQ ID NO: 24 - protein, 7 aa, synthetic construct
NNQTRMM

SEQ ID NO: 25 - protein, 7 aa, synthetic construct
TNPDRSA

SEQ ID NO: 26 - protein, 7 aa, synthetic construct
ANGTKNG

SEQ ID NO: 27 - protein, 7 aa, synthetic construct
GNETRYT

SEQ ID NO: 28 - protein, 7 aa, synthetic construct
NNSDKVK

SEQ ID NO: 29 - protein, 7 aa, synthetic construct
TNQTKVH

SEQ ID NO: 30 - protein, 7 aa, synthetic construct
ANIVKLQ

SEQ ID NO: 31 - protein, 7 aa, synthetic construct
GNFVKSG

SEQ ID NO: 32 - protein, 7 aa, synthetic construct
NNSIKSQ

SEQ ID NO: 33 - protein, 7 aa, synthetic construct
TNSAKPY

SEQ ID NO: 34 - protein, 7 aa, synthetic construct
ANLAKVM

SEQ ID NO: 35 - protein, 7 aa, synthetic construct
GNHSKIQ

SEQ ID NO: 36 - protein, 7 aa, synthetic construct
NNSTRTA

SEQ ID NO: 37 - protein, 7 aa, synthetic construct
TNSTKGA

SEQ ID NO: 38 - protein, 7 aa, synthetic construct
ANLVKTT

SEQ ID NO: 39 - protein, 7 aa, synthetic construct
GNIIRVH

SEQ ID NO: 40 - protein, 7 aa, synthetic construct
NNTIKNG

SEQ ID NO: 41 - protein, 7 aa, synthetic construct
TNSTRIG

SEQ ID NO: 42 - protein, 7 aa, synthetic construct
ANPNKSV

SEQ ID NO: 43 - protein, 7 aa, synthetic construct
GNLPKIA

SEQ ID NO: 44 - protein, 7 aa, synthetic construct
NNVVKNI

SEQ ID NO: 45 - protein, 7 aa, synthetic construct
TNSVKAH

SEQ ID NO: 46 - protein, 7 aa, synthetic construct
ANSERFL

SEQ ID NO: 47 - protein, 7 aa, synthetic construct
GNNIRTQ

SEQ ID NO: 48 - protein, 7 aa, synthetic construct
NNYTKHQ

SEQ ID NO: 49 - protein, 7 aa, synthetic construct
TNTLKNV

SEQ ID NO: 50 - protein, 7 aa, synthetic construct
ANSIRDV

SEQ ID NO: 51 - protein, 7 aa, synthetic construct
GNNSKPP

SEQ ID NO: 52 - protein, 7 aa, synthetic construct
NNYTRPI

SEQ ID NO: 53 - protein, 7 aa, synthetic construct
TNTTRSP

SEQ ID NO: 54 - protein, 7 aa, synthetic construct
ANSTRTW

SEQ ID NO: 55 - protein, 7 aa, synthetic construct
GNNTKMG

SEQ ID NO: 56 - protein, 7 aa, synthetic construct
PNALRMV

SEQ ID NO: 57 - protein, 7 aa, synthetic construct
VNAIRDY

SEQ ID NO: 58 - protein, 7 aa, synthetic construct
ANTTKIE

SEQ ID NO: 59 - protein, 7 aa, synthetic construct
GNPEKND

SEQ ID NO: 60 - protein, 7 aa, synthetic construct
PNEQKVI

SEQ ID NO: 61 - protein, 7 aa, synthetic construct
VNATRVY

SEQ ID NO: 62 - protein, 7 aa, synthetic construct
ANVVKQF

SEQ ID NO: 63 - protein, 7 aa, synthetic construct
GNQTKNG

SEQ ID NO: 64 - protein, 7 aa, synthetic construct
PNIIKQN

SEQ ID NO: 65 - protein, 7 aa, synthetic construct
VNGGKFM

SEQ ID NO: 66 - protein, 7 aa, synthetic construct
CNEGRSC

SEQ ID NO: 67 - protein, 7 aa, synthetic construct
GNQTRGG

SEQ ID NO: 68 - protein, 7 aa, synthetic construct
PNLSKER

SEQ ID NO: 69 - protein, 7 aa, synthetic construct
VNITKNF

SEQ ID NO: 70 - protein, 7 aa, synthetic construct
DNDHRDI

SEQ ID NO: 71 - protein, 7 aa, synthetic construct
GNQVKGG

SEQ ID NO: 72 - protein, 7 aa, synthetic construct
PNMQKQI

SEQ ID NO: 73 - protein, 7 aa, synthetic construct
VNITKSW

SEQ ID NO: 74 - protein, 7 aa, synthetic construct
DNDSKSS

SEQ ID NO: 75 - protein, 7 aa, synthetic construct
GNRSKDY

SEQ ID NO: 76 - protein, 7 aa, synthetic construct
PNSDKLK

SEQ ID NO: 77 - protein, 7 aa, synthetic construct
VNMTKPI

SEQ ID NO: 78 - protein, 7 aa, synthetic construct
DNDVKGG

SEQ ID NO: 79 - protein, 7 aa, synthetic construct
GNSVKNG

SEQ ID NO: 80 - protein, 7 aa, synthetic construct
PNVAKQQ

SEQ ID NO: 81 - protein, 7 aa, synthetic construct
VNMVRTF

SEQ ID NO: 82 - protein, 7 aa, synthetic construct
DNGTKTF

SEQ ID NO: 83 - protein, 7 aa, synthetic construct
GNTIRQA

SEQ ID NO: 84 - protein, 7 aa, synthetic construct
QNALRNI

SEQ ID NO: 85 - protein, 7 aa, synthetic construct
VNQVKNH

SEQ ID NO: 86 - protein, 7 aa, synthetic construct
DNGVRGY

SEQ ID NO: 87 - protein, 7 aa, synthetic construct
GNTVKGS

SEQ ID NO: 88 - protein, 7 aa, synthetic construct
QNEVRNG

SEQ ID NO: 89 - protein, 7 aa, synthetic construct
VNVERKL

SEQ ID NO: 90 - protein, 7 aa, synthetic construct
DNHIKPS

SEQ ID NO: 91 - protein, 7 aa, synthetic construct
GNVVKST

SEQ ID NO: 92 - protein, 7 aa, synthetic construct
QNFVKIS

SEQ ID NO: 93 - protein, 7 aa, synthetic construct
VNVGKGP

SEQ ID NO: 94 - protein, 7 aa, synthetic construct
DNIFKEF

SEQ ID NO: 95 - protein, 7 aa, synthetic construct
HNANKML

SEQ ID NO: 96 - protein, 7 aa, synthetic construct
QNLGKFI

SEQ ID NO: 97 - protein, 7 aa, synthetic construct
WNGNRAA

SEQ ID NO: 98 - protein, 7 aa, synthetic construct
DNKIKST

SEQ ID NO: 99 - protein, 7 aa, synthetic construct
HNEPKNH

SEQ ID NO: 100 - protein, 7 aa, synthetic construct
QNMDKPA

SEQ ID NO: 101 - protein, 7 aa, synthetic construct
YNATRGG

SEQ ID NO: 102 - protein, 7 aa, synthetic construct
DNKLRPI

SEQ ID NO: 103 - protein, 7 aa, synthetic construct
HNLDKTH

SEQ ID NO: 104 - protein, 7 aa, synthetic construct
QNMVRGN

SEQ ID NO: 105 - protein, 7 aa, synthetic construct
YNSTRNG

SEQ ID NO: 106 - protein, 7 aa, synthetic construct
DNMIRKV

SEQ ID NO: 107 - protein, 7 aa, synthetic construct
HNLNKFT

SEQ ID NO: 108 - protein, 7 aa, synthetic construct
QNQTRIQ

SEQ ID NO: 109 - protein, 7 aa, synthetic construct
YNTQKEI

SEQ ID NO: 110 - protein, 7 aa, synthetic construct
DNPSRSV

SEQ ID NO: 111 - protein, 7 aa, synthetic construct
HNPIRFP

SEQ ID NO: 112 - protein, 7 aa, synthetic construct
QNTIRMY

SEQ ID NO: 113 - protein, 7 aa, synthetic construct
YNTTKDK

SEQ ID NO: 114 - protein, 7 aa, synthetic construct
DNQMKFV

SEQ ID NO: 115 - protein, 7 aa, synthetic construct
INKEKFT

SEQ ID NO: 116 - protein, 7 aa, synthetic construct
QNTTKAG

SEQ ID NO: 117 - protein, 7 aa, synthetic construct
DNTERIP

SEQ ID NO: 118 - protein, 7 aa, synthetic construct
INSDKSL

SEQ ID NO: 119 - protein, 7 aa, synthetic construct
QNYTKNN

SEQ ID NO: 120 - protein, 7 aa, synthetic construct
DNTERPA

SEQ ID NO: 121 - protein, 7 aa, synthetic construct
KNDLRNE

SEQ ID NO: 122 - protein, 7 aa, synthetic construct
RNDTKGG

SEQ ID NO: 123 - protein, 7 aa, synthetic construct
DNTNKEE

SEQ ID NO: 124 - protein, 7 aa, synthetic construct
KNEPKNL

SEQ ID NO: 125 - protein, 7 aa, synthetic construct
SNAIKLS

SEQ ID NO: 126 - protein, 7 aa, synthetic construct
ENADKGR

SEQ ID NO: 127 - protein, 7 aa, synthetic construct
KNEVKNA

SEQ ID NO: 128 - protein, 7 aa, synthetic construct
SNAPRIV

SEQ ID NO: 129 - protein, 7 aa, synthetic construct
ENDPKAV

SEQ ID NO: 130 - protein, 7 aa, synthetic construct
LNAMRPL

SEQ ID NO: 131 - protein, 7 aa, synthetic construct
SNAPRQV

SEQ ID NO: 132 - protein, 7 aa, synthetic construct
ENGLKYP

SEQ ID NO: 133 - protein, 7 aa, synthetic construct
LNGERQI

SEQ ID NO: 134 - protein, 7 aa, synthetic construct
SNAVKSA

SEQ ID NO: 135 - protein, 7 aa, synthetic construct
ENHVKGQ

SEQ ID NO: 136 - protein, 7 aa, synthetic construct
LNKDKLI

SEQ ID NO: 137 - protein, 7 aa, synthetic construct
SNDIRNN

SEQ ID NO: 138 - protein, 7 aa, synthetic construct
ENKERDV

SEQ ID NO: 139 - protein, 7 aa, synthetic construct
LNLNRLE

SEQ ID NO: 140 - protein, 7 aa, synthetic construct
SNDKRPL

SEQ ID NO: 141 - protein, 7 aa, synthetic construct
ENKIKLV

SEQ ID NO: 142 - protein, 7 aa, synthetic construct
MNGSKTA

SEQ ID NO: 143 - protein, 7 aa, synthetic construct
SNHIKSA

SEQ ID NO: 144 - protein, 7 aa, synthetic construct
ENLERLK

SEQ ID NO: 145 - protein, 7 aa, synthetic construct
MNQQKLV

SEQ ID NO: 146 - protein, 7 aa, synthetic construct
SNHTKNF

SEQ ID NO: 147 - protein, 7 aa, synthetic construct
ENNVKAV

SEQ ID NO: 148 - protein, 7 aa, synthetic construct
MNQTRGG

SEQ ID NO: 149 - protein, 7 aa, synthetic construct
SNLQKFI

SEQ ID NO: 150 - protein, 7 aa, synthetic construct
ENPIKST

SEQ ID NO: 151 - protein, 7 aa, synthetic construct
MNSTKNG

SEQ ID NO: 152 - protein, 7 aa, synthetic construct
SNLQKVE

SEQ ID NO: 153 - protein, 7 aa, synthetic construct
ENQIRSL

SEQ ID NO: 154 - protein, 7 aa, synthetic construct
MNSVRFM

SEQ ID NO: 155 - protein, 7 aa, synthetic construct
SNQVRAL

SEQ ID NO: 156 - protein, 7 aa, synthetic construct
ENQVKQY

SEQ ID NO: 157 - protein, 7 aa, synthetic construct
MNVTKQT

SEQ ID NO: 158 - protein, 7 aa, synthetic construct
SNSTKSA

SEQ ID NO: 159 - protein, 7 aa, synthetic construct
ENQVRME

SEQ ID NO: 160 - protein, 7 aa, synthetic construct
NNATRAA

SEQ ID NO: 161 - protein, 7 aa, synthetic construct
SNSTRAP

SEQ ID NO: 162 - protein, 7 aa, synthetic construct
ENQYKAI

SEQ ID NO: 163 - protein, 7 aa, synthetic construct
NNATRPA

SEQ ID NO: 164 - protein, 7 aa, synthetic construct
SNTTRPI

SEQ ID NO: 165 - protein, 7 aa, synthetic construct
ENTSRTQ

SEQ ID NO: 166 - protein, 7 aa, synthetic construct
NNGTRLS

SEQ ID NO: 167 - protein, 7 aa, synthetic construct
SNVNRLV

SEQ ID NO: 168 - protein, 7 aa, synthetic construct
ENVERLK

SEQ ID NO: 169 - protein, 7 aa, synthetic construct
NNHDKSK

SEQ ID NO: 170 - protein, 7 aa, synthetic construct
SNVTKWV

SEQ ID NO: 171 - protein, 7 aa, synthetic construct
ENVIRSV

SEQ ID NO: 172 - protein, 7 aa, synthetic construct
NNHIRET

SEQ ID NO: 173 - protein, 7 aa, synthetic construct
TNATKGG

SEQ ID NO: 174 - protein, 7 aa, synthetic construct
FNAERMT

SEQ ID NO: 175 - protein, 7 aa, synthetic construct
NNHIRYH

SEQ ID NO: 176 - protein, 7 aa, synthetic construct
TNDDKYQ

SEQ ID NO: 177 - protein, 7 aa, synthetic construct
FNDAKGT

SEQ ID NO: 178 - protein, 7 aa, synthetic construct
NNHTKDI

SEQ ID NO: 179 - protein, 7 aa, synthetic construct
TNDVRRL

SEQ ID NO: 180 - protein, 7 aa, synthetic construct
FNDAKQW

SEQ ID NO: 181 - protein, 7 aa, synthetic construct
NNHTKGV

SEQ ID NO: 182 - protein, 7 aa, synthetic construct
TNEDKGL

SEQ ID NO: 183 - protein, 7 aa, synthetic construct
ANAVRAI

SEQ ID NO: 184 - protein, 7 aa, synthetic construct
GNSIRDT

SEQ ID NO: 185 - protein, 7 aa, synthetic construct
LNSSRIP

SEQ ID NO: 186 - protein, 7 aa, synthetic construct
SNAIKST

SEQ ID NO: 187 - protein, 7 aa, synthetic construct
ANDIRPW

SEQ ID NO: 188 - protein, 7 aa, synthetic construct
GNSIRTI

SEQ ID NO: 189 - protein, 7 aa, synthetic construct
LNSVRHV

SEQ ID NO: 190 - protein, 7 aa, synthetic construct
SNATRQV

SEQ ID NO: 191 - protein, 7 aa, synthetic construct
ANHIRSL

SEQ ID NO: 192 - protein, 7 aa, synthetic construct
GNSLRAY

SEQ ID NO: 193 - protein, 7 aa, synthetic construct
LNTIRST

SEQ ID NO: 194 - protein, 7 aa, synthetic construct
SNATRSI

SEQ ID NO: 195 - protein, 7 aa, synthetic construct
ANTGRHN

SEQ ID NO: 196 - protein, 7 aa, synthetic construct
GNSMRHM

SEQ ID NO: 197 - protein, 7 aa, synthetic construct
LNTIRTN

SEQ ID NO: 198 - protein, 7 aa, synthetic construct
SNEIRLV

SEQ ID NO: 199 - protein, 7 aa, synthetic construct
ANYARND

SEQ ID NO: 200 - protein, 7 aa, synthetic construct
GNSSRNL

SEQ ID NO: 201 - protein, 7 aa, synthetic construct
LNTTRPI

SEQ ID NO: 202 - protein, 7 aa, synthetic construct
SNFIRSA

SEQ ID NO: 203 - protein, 7 aa, synthetic construct
DNAVRPW

SEQ ID NO: 204 - protein, 7 aa, synthetic construct
GNSTRDH

SEQ ID NO: 205 - protein, 7 aa, synthetic construct
LNTTRQV

SEQ ID NO: 206 - protein, 7 aa, synthetic construct
SNHIRLA

SEQ ID NO: 207 - protein, 7 aa, synthetic construct
DNEIRRW

SEQ ID NO: 208 - protein, 7 aa, synthetic construct
GNSVKPS

SEQ ID NO: 209 - protein, 7 aa, synthetic construct
LNTVRDI

SEQ ID NO: 210 - protein, 7 aa, synthetic construct
SNHIRTL

SEQ ID NO: 211 - protein, 7 aa, synthetic construct
DNHIRSQ

SEQ ID NO: 212 - protein, 7 aa, synthetic construct
GNSVKQF

SEQ ID NO: 213 - protein, 7 aa, synthetic construct
LNTVREP

SEQ ID NO: 214 - protein, 7 aa, synthetic construct
SNHVRAI

SEQ ID NO: 215 - protein, 7 aa, synthetic construct
DNHVKSL

SEQ ID NO: 216 - protein, 7 aa, synthetic construct
GNSVRDF

SEQ ID NO: 217 - protein, 7 aa, synthetic construct
LNTVRHI

SEQ ID NO: 218 - protein, 7 aa, synthetic construct
SNHVRFM

SEQ ID NO: 219 - protein, 7 aa, synthetic construct
DNKVKPT

SEQ ID NO: 220 - protein, 7 aa, synthetic construct
GNSVRGW

SEQ ID NO: 221 - protein, 7 aa, synthetic construct
LNVIRDP

SEQ ID NO: 222 - protein, 7 aa, synthetic construct
SNHVRTS

SEQ ID NO: 223 - protein, 7 aa, synthetic construct
DNNTRSV

SEQ ID NO: 224 - protein, 7 aa, synthetic construct
GNSVRPV

SEQ ID NO: 225 - protein, 7 aa, synthetic construct
LNYTRSS

SEQ ID NO: 226 - protein, 7 aa, synthetic construct
SNNGRYM

SEQ ID NO: 227 - protein, 7 aa, synthetic construct
DNRTKAL

SEQ ID NO: 228 - protein, 7 aa, synthetic construct
GNTIRDI

SEQ ID NO: 229 - protein, 7 aa, synthetic construct
MNDVRSV

SEQ ID NO: 230 - protein, 7 aa, synthetic construct
SNNVRHY

SEQ ID NO: 231 - protein, 7 aa, synthetic construct
DNRVRSP

SEQ ID NO: 232 - protein, 7 aa, synthetic construct
GNTTKST

SEQ ID NO: 233 - protein, 7 aa, synthetic construct
MNHIRTT

SEQ ID NO: 234 - protein, 7 aa, synthetic construct
SNQTRSV

SEQ ID NO: 235 - protein, 7 aa, synthetic construct
DNSTRAT

SEQ ID NO: 236 - protein, 7 aa, synthetic construct
GNTVKDP

SEQ ID NO: 237 - protein, 7 aa, synthetic construct
MNNARPY

SEQ ID NO: 238 - protein, 7 aa, synthetic construct
SNSTKIL

SEQ ID NO: 239 - protein, 7 aa, synthetic construct
DNSTRWS

SEQ ID NO: 240 - protein, 7 aa, synthetic construct
GNTVKLL

SEQ ID NO: 241 - protein, 7 aa, synthetic construct
MNQARNP

SEQ ID NO: 242 - protein, 7 aa, synthetic construct
SNSTRSV

SEQ ID NO: 243 - protein, 7 aa, synthetic construct
DNSVRLT

SEQ ID NO: 244 - protein, 7 aa, synthetic construct
GNTVRDT

SEQ ID NO: 245 - protein, 7 aa, synthetic construct
MNTIRSY

SEQ ID NO: 246 - protein, 7 aa, synthetic construct
SNSVRAM

SEQ ID NO: 247 - protein, 7 aa, synthetic construct
DNTVRGS

SEQ ID NO: 248 - protein, 7 aa, synthetic construct
GNVIKTW

SEQ ID NO: 249 - protein, 7 aa, synthetic construct
MNTVRDY

SEQ ID NO: 250 - protein, 7 aa, synthetic construct
SNSVRDR

SEQ ID NO: 251 - protein, 7 aa, synthetic construct
DNTWKAA

SEQ ID NO: 252 - protein, 7 aa, synthetic construct
GNVIRSH

SEQ ID NO: 253 - protein, 7 aa, synthetic construct
NNALKPY

SEQ ID NO: 254 - protein, 7 aa, synthetic construct
SNTIKNL

SEQ ID NO: 255 - protein, 7 aa, synthetic construct
ENATRSL

SEQ ID NO: 256 - protein, 7 aa, synthetic construct
GNVTRST

SEQ ID NO: 257 - protein, 7 aa, synthetic construct
NNASKGW

SEQ ID NO: 258 - protein, 7 aa, synthetic construct
SNTIRTI

SEQ ID NO: 259 - protein, 7 aa, synthetic construct
ENHLRNT

SEQ ID NO: 260 - protein, 7 aa, synthetic construct
GNYGRAQ

SEQ ID NO: 261 - protein, 7 aa, synthetic construct
NNATRNG

SEQ ID NO: 262 - protein, 7 aa, synthetic construct
SNTTKAG

SEQ ID NO: 263 - protein, 7 aa, synthetic construct
ENHVRNM

SEQ ID NO: 264 - protein, 7 aa, synthetic construct
HNMSRIA

SEQ ID NO: 265 - protein, 7 aa, synthetic construct
NNFTRGT

SEQ ID NO: 266 - protein, 7 aa, synthetic construct
TNAVRGT

SEQ ID NO: 267 - protein, 7 aa, synthetic construct
ENNTKIT

SEQ ID NO: 268 - protein, 7 aa, synthetic construct
INDARTV

SEQ ID NO: 269 - protein, 7 aa, synthetic construct
NNNGRSL

SEQ ID NO: 270 - protein, 7 aa, synthetic construct
TNELRSY

SEQ ID NO: 271 - protein, 7 aa, synthetic construct
ENPIRGR

SEQ ID NO: 272 - protein, 7 aa, synthetic construct
INESRNR

SEQ ID NO: 273 - protein, 7 aa, synthetic construct
NNSARPL

SEQ ID NO: 274 - protein, 7 aa, synthetic construct
TNEVRLS

SEQ ID NO: 275 - protein, 7 aa, synthetic construct
ENRIRDN

SEQ ID NO: 276 - protein, 7 aa, synthetic construct
INHLKSA

SEQ ID NO: 277 - protein, 7 aa, synthetic construct
NNSTRAG

SEQ ID NO: 278 - protein, 7 aa, synthetic construct
TNFTKMT

SEQ ID NO: 279 - protein, 7 aa, synthetic construct
ENSIKPL

SEQ ID NO: 280 - protein, 7 aa, synthetic construct
INNERSK

SEQ ID NO: 281 - protein, 7 aa, synthetic construct
NNSTRGG

SEQ ID NO: 282 - protein, 7 aa, synthetic construct
TNGTKPT

SEQ ID NO: 283 - protein, 7 aa, synthetic construct
ENSVRSA

SEQ ID NO: 284 - protein, 7 aa, synthetic construct
INNVKSA

SEQ ID NO: 285 - protein, 7 aa, synthetic construct
NNSTRGL

SEQ ID NO: 286 - protein, 7 aa, synthetic construct
TNGTRNL

SEQ ID NO: 287 - protein, 7 aa, synthetic construct
ENVVRSK

SEQ ID NO: 288 - protein, 7 aa, synthetic construct
INNVRSV

SEQ ID NO: 289 - protein, 7 aa, synthetic construct
NNTIRYS

SEQ ID NO: 290 - protein, 7 aa, synthetic construct
TNHIRFT

SEQ ID NO: 291 - protein, 7 aa, synthetic construct
GNATKAT

SEQ ID NO: 292 - protein, 7 aa, synthetic construct
INPRKDN

SEQ ID NO: 293 - protein, 7 aa, synthetic construct
NNTTRGM

SEQ ID NO: 294 - protein, 7 aa, synthetic construct
TNHIRHL

SEQ ID NO: 295 - protein, 7 aa, synthetic construct
GNAVKST

SEQ ID NO: 296 - protein, 7 aa, synthetic construct
INQIRAA

SEQ ID NO: 297 - protein, 7 aa, synthetic construct
NNTVRGM

SEQ ID NO: 298 - protein, 7 aa, synthetic construct
TNHIRNL

SEQ ID NO: 299 - protein, 7 aa, synthetic construct
GNAVRTG

SEQ ID NO: 300 - protein, 7 aa, synthetic construct
INQIRQV

SEQ ID NO: 301 - protein, 7 aa, synthetic construct
NNVIRGF

SEQ ID NO: 302 - protein, 7 aa, synthetic construct
TNHSRPV

SEQ ID NO: 303 - protein, 7 aa, synthetic construct
GNAVRTV

SEQ ID NO: 304 - protein, 7 aa, synthetic construct
INSIRDL

SEQ ID NO: 305 - protein, 7 aa, synthetic construct
PNDIRLR

SEQ ID NO: 306 - protein, 7 aa, synthetic construct
TNMIRNA

SEQ ID NO: 307 - protein, 7 aa, synthetic construct
GNDIRVR

SEQ ID NO: 308 - protein, 7 aa, synthetic construct
INSIRMT

SEQ ID NO: 309 - protein, 7 aa, synthetic construct
PNGIKGV

SEQ ID NO: 310 - protein, 7 aa, synthetic construct
TNMPRNS

SEQ ID NO: 311 - protein, 7 aa, synthetic construct
GNDIRYY

SEQ ID NO: 312 - protein, 7 aa, synthetic construct
INTVRDR

SEQ ID NO: 313 - protein, 7 aa, synthetic construct
PNNLRTP

SEQ ID NO: 314 - protein, 7 aa, synthetic construct
TNPSRFA

SEQ ID NO: 315 - protein, 7 aa, synthetic construct
GNDTRST

SEQ ID NO: 316 - protein, 7 aa, synthetic construct
INYQKPA

SEQ ID NO: 317 - protein, 7 aa, synthetic construct
PNNVRQY

SEQ ID NO: 318 - protein, 7 aa, synthetic construct
TNSIRDQ

SEQ ID NO: 319 - protein, 7 aa, synthetic construct
GNESRPT

SEQ ID NO: 320 - protein, 7 aa, synthetic construct
INYVKHH

SEQ ID NO: 321 - protein, 7 aa, synthetic construct
PNSIRRD

SEQ ID NO: 322 - protein, 7 aa, synthetic construct
TNSTKAG

SEQ ID NO: 323 - protein, 7 aa, synthetic construct
GNESRYM

SEQ ID NO: 324 - protein, 7 aa, synthetic construct
KNLLRSE

SEQ ID NO: 325 - protein, 7 aa, synthetic construct
PNSMRQV

SEQ ID NO: 326 - protein, 7 aa, synthetic construct
TNSTRFT

SEQ ID NO: 327 - protein, 7 aa, synthetic construct
GNEVRYY

SEQ ID NO: 328 - protein, 7 aa, synthetic construct
LNDLRSR

SEQ ID NO: 329 - protein, 7 aa, synthetic construct
PNSTRNV

SEQ ID NO: 330 - protein, 7 aa, synthetic construct
TNSTRGV

SEQ ID NO: 331 - protein, 7 aa, synthetic construct
GNFTRDL

SEQ ID NO: 332 - protein, 7 aa, synthetic construct
LNEIRAV

SEQ ID NO: 333 - protein, 7 aa, synthetic construct
PNTIRNV

SEQ ID NO: 334 - protein, 7 aa, synthetic construct
TNSVRPV

SEQ ID NO: 335 - protein, 7 aa, synthetic construct
GNGTRVY

SEQ ID NO: 336 - protein, 7 aa, synthetic construct
LNEPRRV

SEQ ID NO: 337 - protein, 7 aa, synthetic construct
PNTTRYL

SEQ ID NO: 338 - protein, 7 aa, synthetic construct
TNTVRGG

SEQ ID NO: 339 - protein, 7 aa, synthetic construct
GNHVRDA

SEQ ID NO: 340 - protein, 7 aa, synthetic construct
LNEVKLY

SEQ ID NO: 341 - protein, 7 aa, synthetic construct
QNDVRYP

SEQ ID NO: 342 - protein, 7 aa, synthetic construct
TNVVKQT

SEQ ID NO: 343 - protein, 7 aa, synthetic construct
GNLGRSS

SEQ ID NO: 344 - protein, 7 aa, synthetic construct
LNHMRNT

SEQ ID NO: 345 - protein, 7 aa, synthetic construct
QNEIKTY

SEQ ID NO: 346 - protein, 7 aa, synthetic construct
TNYTKTL

SEQ ID NO: 347 - protein, 7 aa, synthetic construct
GNLTKGY

SEQ ID NO: 348 - protein, 7 aa, synthetic construct
LNNIRNV

SEQ ID NO: 349 - protein, 7 aa, synthetic construct
QNKHREM

SEQ ID NO: 350 - protein, 7 aa, synthetic construct
VNATRGG

SEQ ID NO: 351 - protein, 7 aa, synthetic construct
GNMIRND

SEQ ID NO: 352 - protein, 7 aa, synthetic construct
LNNIRQV

SEQ ID NO: 353 - protein, 7 aa, synthetic construct
QNNIKAW

SEQ ID NO: 354 - protein, 7 aa, synthetic construct
VNATRST

SEQ ID NO: 355 - protein, 7 aa, synthetic construct
GNNTKAV

SEQ ID NO: 356 - protein, 7 aa, synthetic construct
LNNVRPT

SEQ ID NO: 357 - protein, 7 aa, synthetic construct
QNRMRND

SEQ ID NO: 358 - protein, 7 aa, synthetic construct
VNEVRFQ

SEQ ID NO: 359 - protein, 7 aa, synthetic construct
GNNTRSA

SEQ ID NO: 360 - protein, 7 aa, synthetic construct
LNNVRSP

SEQ ID NO: 361 - protein, 7 aa, synthetic construct
QNRQRDT

SEQ ID NO: 362 - protein, 7 aa, synthetic construct
VNHDRAR

SEQ ID NO: 363 - protein, 7 aa, synthetic construct
GNNVKGL

SEQ ID NO: 364 - protein, 7 aa, synthetic construct
LNNVRSV

SEQ ID NO: 365 - protein, 7 aa, synthetic construct
RNDGRVA

SEQ ID NO: 366 - protein, 7 aa, synthetic construct
VNHIRLQ

SEQ ID NO: 367 - protein, 7 aa, synthetic construct
GNNVKQF

SEQ ID NO: 368 - protein, 7 aa, synthetic construct
LNQYRAA

SEQ ID NO: 369 - protein, 7 aa, synthetic construct
RNDTRHI

SEQ ID NO: 370 - protein, 7 aa, synthetic construct
VNHLREV

SEQ ID NO: 371 - protein, 7 aa, synthetic construct
GNQTKPF

SEQ ID NO: 372 - protein, 7 aa, synthetic construct
LNRVRGD

SEQ ID NO: 373 - protein, 7 aa, synthetic construct
RNERRDV

SEQ ID NO: 374 - protein, 7 aa, synthetic construct
VNHVRLT

SEQ ID NO: 375 - protein, 7 aa, synthetic construct
GNRVKET

SEQ ID NO: 376 - protein, 7 aa, synthetic construct
LNSARSI

SEQ ID NO: 377 - protein, 7 aa, synthetic construct
RNEVRFA

SEQ ID NO: 378 - protein, 7 aa, synthetic construct
VNNERSK

SEQ ID NO: 379 - protein, 7 aa, synthetic construct
GNSARNI

SEQ ID NO: 380 - protein, 7 aa, synthetic construct
LNSGRSA

SEQ ID NO: 381 - protein, 7 aa, synthetic construct
RNTVKDP

SEQ ID NO: 382 - protein, 7 aa, synthetic construct
VNTIRNV

SEQ ID NO: 383 - protein, 7 aa, synthetic construct
GNSIRAL

SEQ ID NO: 384 - protein, 7 aa, synthetic construct
LNSPRNV

SEQ ID NO: 385 - protein, 7 aa, synthetic construct
SNAIKAT

SEQ ID NO: 386 - protein, 7 aa, synthetic construct
VNTIRSV

SEQ ID NO: 387 - protein, 7 aa, synthetic construct
YNQMRNT

SEQ ID NO: 388 - protein, 7 aa, synthetic construct
ANAERQP

SEQ ID NO: 389 - protein, 7 aa, synthetic construct
GNETKSV

SEQ ID NO: 390 - protein, 7 aa, synthetic construct
NNMVRTP

SEQ ID NO: 391 - protein, 7 aa, synthetic construct
VNTVREF

SEQ ID NO: 392 - protein, 7 aa, synthetic construct
ANDMRSG

SEQ ID NO: 393 - protein, 7 aa, synthetic construct
GNETRML

SEQ ID NO: 394 - protein, 7 aa, synthetic construct
NNSIKVG

SEQ ID NO: 395 - protein, 7 aa, synthetic construct
VNTVREF

SEQ ID NO: 396 - protein, 7 aa, synthetic construct
ANDSRAT

SEQ ID NO: 397 - protein, 7 aa, synthetic construct
GNGTRLN

SEQ ID NO: 398 - protein, 7 aa, synthetic construct
NNSLKEK

SEQ ID NO: 399 - protein, 7 aa, synthetic construct
WNANRIN

SEQ ID NO: 400 - protein, 7 aa, synthetic construct
ANGTKII

SEQ ID NO: 401 - protein, 7 aa, synthetic construct
GNHVKQD

SEQ ID NO: 402 - protein, 7 aa, synthetic construct
NNTVKNW

SEQ ID NO: 403 - protein, 7 aa, synthetic construct
WNENKSM

SEQ ID NO: 404 - protein, 7 aa, synthetic construct
ANITKSI

SEQ ID NO: 405 - protein, 7 aa, synthetic construct
GNISKNI

SEQ ID NO: 406 - protein, 7 aa, synthetic construct
NNVSRDS

SEQ ID NO: 407 - protein, 7 aa, synthetic construct
YNAAKGV

SEQ ID NO: 408 - protein, 7 aa, synthetic construct
ANLSKTT

SEQ ID NO: 409 - protein, 7 aa, synthetic construct
GNLVKEN

SEQ ID NO: 410 - protein, 7 aa, synthetic construct
NNVSRNN

SEQ ID NO: 411 - protein, 7 aa, synthetic construct
YNDDRVF

SEQ ID NO: 412 - protein, 7 aa, synthetic construct
DNAHRTQ

SEQ ID NO: 413 - protein, 7 aa, synthetic construct
GNNIRNY

SEQ ID NO: 414 - protein, 7 aa, synthetic construct
PNASRDW

SEQ ID NO: 415 - protein, 7 aa, synthetic construct
YNERKEV

SEQ ID NO: 416 - protein, 7 aa, synthetic construct
DNDIRGK

SEQ ID NO: 417 - protein, 7 aa, synthetic construct
GNSARAV

SEQ ID NO: 418 - protein, 7 aa, synthetic construct
PNNNKNH

SEQ ID NO: 419 - protein, 7 aa, synthetic construct
YNESRNL

SEQ ID NO: 420 - protein, 7 aa, synthetic construct
DNESKKS

SEQ ID NO: 421 - protein, 7 aa, synthetic construct
GNVVKHM

SEQ ID NO: 422 - protein, 7 aa, synthetic construct
PNTSKFT

SEQ ID NO: 423 - protein, 7 aa, synthetic construct
YNEVKAN

SEQ ID NO: 424 - protein, 7 aa, synthetic construct
DNKTRLT

SEQ ID NO: 425 - protein, 7 aa, synthetic construct
GNYVRDH

SEQ ID NO: 426 - protein, 7 aa, synthetic construct
QNALRST

SEQ ID NO: 427 - protein, 7 aa, synthetic construct
YNHQKHD

SEQ ID NO: 428 - protein, 7 aa, synthetic construct
DNNGRLT

SEQ ID NO: 429 - protein, 7 aa, synthetic construct
HNLDRSN

SEQ ID NO: 430 - protein, 7 aa, synthetic construct
QNALRST

SEQ ID NO: 431 - protein, 7 aa, synthetic construct
YNLSRDD

SEQ ID NO: 432 - protein, 7 aa, synthetic construct
DNNVRNV

SEQ ID NO: 433 - protein, 7 aa, synthetic construct
HNLDRSN

SEQ ID NO: 434 - protein, 7 aa, synthetic construct
QNGDKLP

SEQ ID NO: 435 - protein, 7 aa, synthetic construct
YNMGKDH

SEQ ID NO: 436 - protein, 7 aa, synthetic construct
DNPSRGI

SEQ ID NO: 437 - protein, 7 aa, synthetic construct
HNLERQT

SEQ ID NO: 438 - protein, 7 aa, synthetic construct
QNNIKDK

SEQ ID NO: 439 - protein, 7 aa, synthetic construct
YNSIRNN

SEQ ID NO: 440 - protein, 7 aa, synthetic construct
DNPVRGI

SEQ ID NO: 441 - protein, 7 aa, synthetic construct
HNLERQT

SEQ ID NO: 442 - protein, 7 aa, synthetic construct
QNVARNS

SEQ ID NO: 443 - protein, 7 aa, synthetic construct
YNSNKPN

SEQ ID NO: 444 - protein, 7 aa, synthetic construct
DNRHKEG

SEQ ID NO: 445 - protein, 7 aa, synthetic construct
HNLHREN

SEQ ID NO: 446 - protein, 7 aa, synthetic construct
RNADKGI

SEQ ID NO: 447 - protein, 7 aa, synthetic construct
YNTQKYG

SEQ ID NO: 448 - protein, 7 aa, synthetic construct
DNRIRGD

SEQ ID NO: 449 - protein, 7 aa, synthetic construct
INETRNL

SEQ ID NO: 450 - protein, 7 aa, synthetic construct
RNDARNS

SEQ ID NO: 451 - protein, 7 aa, synthetic construct
DNSARET

SEQ ID NO: 452 - protein, 7 aa, synthetic construct
INGDRAR

SEQ ID NO: 453 - protein, 7 aa, synthetic construct
RNGSKSD

SEQ ID NO: 454 - protein, 7 aa, synthetic construct
ENASKAF

SEQ ID NO: 455 - protein, 7 aa, synthetic construct
INGSRNL

SEQ ID NO: 456 - protein, 7 aa, synthetic construct
RNNVKAD

SEQ ID NO: 457 - protein, 7 aa, synthetic construct
ENAVRSG

SEQ ID NO: 458 - protein, 7 aa, synthetic construct
INKEKTI

SEQ ID NO: 459 - protein, 7 aa, synthetic construct
RNTEKEQ

SEQ ID NO: 460 - protein, 7 aa, synthetic construct
ENHLRNT

SEQ ID NO: 461 - protein, 7 aa, synthetic construct
INMSKSA

SEQ ID NO: 462 - protein, 7 aa, synthetic construct
SNDGRSN

SEQ ID NO: 463 - protein, 7 aa, synthetic construct
ENHTRDK

SEQ ID NO: 464 - protein, 7 aa, synthetic construct
INMSKSA

SEQ ID NO: 465 - protein, 7 aa, synthetic construct
SNDQKDN

SEQ ID NO: 466 - protein, 7 aa, synthetic construct
ENHVKQN

SEQ ID NO: 467 - protein, 7 aa, synthetic construct
INSDRGD

SEQ ID NO: 468 - protein, 7 aa, synthetic construct
SNEARSL

SEQ ID NO: 469 - protein, 7 aa, synthetic construct
ENITKNV

SEQ ID NO: 470 - protein, 7 aa, synthetic construct
KNTSRED

SEQ ID NO: 471 - protein, 7 aa, synthetic construct
SNEPRVL

SEQ ID NO: 472 - protein, 7 aa, synthetic construct
ENLERMV

SEQ ID NO: 473 - protein, 7 aa, synthetic construct
LNATRNL

SEQ ID NO: 474 - protein, 7 aa, synthetic construct
SNGDRSR

SEQ ID NO: 475 - protein, 7 aa, synthetic construct
ENLIKQH

SEQ ID NO: 476 - protein, 7 aa, synthetic construct
LNDIRNT

SEQ ID NO: 477 - protein, 7 aa, synthetic construct
SNGGRLQ

SEQ ID NO: 478 - protein, 7 aa, synthetic construct
ENLIRSN

SEQ ID NO: 479 - protein, 7 aa, synthetic construct
LNDTRYY

SEQ ID NO: 480 - protein, 7 aa, synthetic construct
SNGHKLS

SEQ ID NO: 481 - protein, 7 aa, synthetic construct
ENLIRSN

SEQ ID NO: 482 - protein, 7 aa, synthetic construct
LNGARIV

SEQ ID NO: 483 - protein, 7 aa, synthetic construct
SNMNRIT

SEQ ID NO: 484 - protein, 7 aa, synthetic construct
ENLLRSS

SEQ ID NO: 485 - protein, 7 aa, synthetic construct
LNGARTD

SEQ ID NO: 486 - protein, 7 aa, synthetic construct
SNMPRDT

SEQ ID NO: 487 - protein, 7 aa, synthetic construct
ENLMRSS

SEQ ID NO: 488 - protein, 7 aa, synthetic construct
LNGLRAH

SEQ ID NO: 489 - protein, 7 aa, synthetic construct
SNNIRPI

SEQ ID NO: 490 - protein, 7 aa, synthetic construct
ENLVKDD

SEQ ID NO: 491 - protein, 7 aa, synthetic construct
LNGMKGA

SEQ ID NO: 492 - protein, 7 aa, synthetic construct
SNNMRPA

SEQ ID NO: 493 - protein, 7 aa, synthetic construct
ENNPRAT

SEQ ID NO: 494 - protein, 7 aa, synthetic construct
LNGNRYS

SEQ ID NO: 495 - protein, 7 aa, synthetic construct
SNNTKNF

SEQ ID NO: 496 - protein, 7 aa, synthetic construct
ENNTKNY

SEQ ID NO: 497 - protein, 7 aa, synthetic construct
LNGSRGA

SEQ ID NO: 498 - protein, 7 aa, synthetic construct
SNPDRMK

SEQ ID NO: 499 - protein, 7 aa, synthetic construct
ENQHRTF

SEQ ID NO: 500 - protein, 7 aa, synthetic construct
LNGTRSL

SEQ ID NO: 501 - protein, 7 aa, synthetic construct
SNTIKNL

SEQ ID NO: 502 - protein, 7 aa, synthetic construct
ENTIRNT

SEQ ID NO: 503 - protein, 7 aa, synthetic construct
LNLIKNN

SEQ ID NO: 504 - protein, 7 aa, synthetic construct
TNAVKFT

SEQ ID NO: 505 - protein, 7 aa, synthetic construct
ENTIRNT

SEQ ID NO: 506 - protein, 7 aa, synthetic construct
LNMERTT

SEQ ID NO: 507 - protein, 7 aa, synthetic construct
TNAVKTQ

SEQ ID NO: 508 - protein, 7 aa, synthetic construct
ENTMKAH

SEQ ID NO: 509 - protein, 7 aa, synthetic construct
LNNIKNT

SEQ ID NO: 510 - protein, 7 aa, synthetic construct
TNDHKQY

SEQ ID NO: 511 - protein, 7 aa, synthetic construct
ENYIRDK

SEQ ID NO: 512 - protein, 7 aa, synthetic construct
LNSERSY

SEQ ID NO: 513 - protein, 7 aa, synthetic construct
TNGGKYL

SEQ ID NO: 514 - protein, 7 aa, synthetic construct
ENYIRDK

SEQ ID NO: 515 - protein, 7 aa, synthetic construct
LNSLRGE

SEQ ID NO: 516 - protein, 7 aa, synthetic construct
TNGIRNM

SEQ ID NO: 517 - protein, 7 aa, synthetic construct
ENYVRER

SEQ ID NO: 518 - protein, 7 aa, synthetic construct
LNVDRLI

SEQ ID NO: 519 - protein, 7 aa, synthetic construct
TNGIRPW

SEQ ID NO: 520 - protein, 7 aa, synthetic construct
FNDEKHT

SEQ ID NO: 521 - protein, 7 aa, synthetic construct
MNGGKSL

SEQ ID NO: 522 - protein, 7 aa, synthetic construct
TNGMRNQ

SEQ ID NO: 523 - protein, 7 aa, synthetic construct
FNDGRTN

SEQ ID NO: 524 - protein, 7 aa, synthetic construct
MNGTKAL

SEQ ID NO: 525 - protein, 7 aa, synthetic construct
TNGVRNS

SEQ ID NO: 526 - protein, 7 aa, synthetic construct
FNDNKAF

SEQ ID NO: 527 - protein, 7 aa, synthetic construct
MNHSKSA

SEQ ID NO: 528 - protein, 7 aa, synthetic construct
TNKERFN

SEQ ID NO: 529 - protein, 7 aa, synthetic construct
FNGNKMH

SEQ ID NO: 530 - protein, 7 aa, synthetic construct
MNMDRNT

SEQ ID NO: 531 - protein, 7 aa, synthetic construct
TNMVKDW

SEQ ID NO: 532 - protein, 7 aa, synthetic construct
FNGTRNT

SEQ ID NO: 533 - protein, 7 aa, synthetic construct
MNSIRNT

SEQ ID NO: 534 - protein, 7 aa, synthetic construct
TNQTKSI

SEQ ID NO: 535 - protein, 7 aa, synthetic construct
FNNLKID

SEQ ID NO: 536 - protein, 7 aa, synthetic construct
MNSLRSD

SEQ ID NO: 537 - protein, 7 aa, synthetic construct
TNVVKAG

SEQ ID NO: 538 - protein, 7 aa, synthetic construct
FNQMKNV

SEQ ID NO: 539 - protein, 7 aa, synthetic construct
MNSTKIW

SEQ ID NO: 540 - protein, 7 aa, synthetic construct
TNYTKDP

SEQ ID NO: 541 - protein, 7 aa, synthetic construct
FNSDKSR

SEQ ID NO: 542 - protein, 7 aa, synthetic construct
NNAPKSS

SEQ ID NO: 543 - protein, 7 aa, synthetic construct
TNYTKDP

SEQ ID NO: 544 - protein, 7 aa, synthetic construct
FNTHREG

SEQ ID NO: 545 - protein, 7 aa, synthetic construct
NNGMRSS

SEQ ID NO: 546 - protein, 7 aa, synthetic construct
VNAIRSQ

SEQ ID NO: 547 - protein, 7 aa, synthetic construct
FNVTRVQ

SEQ ID NO: 548 - protein, 7 aa, synthetic construct
NNHMRHD

SEQ ID NO: 549 - protein, 7 aa, synthetic construct
VNEVRNY

SEQ ID NO: 550 - protein, 7 aa, synthetic construct
GNATKDQ

SEQ ID NO: 551 - protein, 7 aa, synthetic construct
NNLTRSL

SEQ ID NO: 552 - protein, 7 aa, synthetic construct
VNSNKHD

SEQ ID NO: 553 - protein, 7 aa, synthetic construct
ANHIRSL

SEQ ID NO: 554 - protein, 7 aa, synthetic construct
INHIRML

SEQ ID NO: 555 - protein, 7 aa, synthetic construct
LNYTRSS

SEQ ID NO: 556 - protein, 7 aa, synthetic construct
SNLRRTE

SEQ ID NO: 557 - protein, 7 aa, synthetic construct
ANLARVT

SEQ ID NO: 558 - protein, 7 aa, synthetic construct
INHLKSA

SEQ ID NO: 559 - protein, 7 aa, synthetic construct
MNEVRYA

SEQ ID NO: 560 - protein, 7 aa, synthetic construct
SNMPRNN

SEQ ID NO: 561 - protein, 7 aa, synthetic construct
ANTGRHN

SEQ ID NO: 562 - protein, 7 aa, synthetic construct
INHMRGA

SEQ ID NO: 563 - protein, 7 aa, synthetic construct
MNHIRTT

SEQ ID NO: 564 - protein, 7 aa, synthetic construct
SNNGRYM

SEQ ID NO: 565 - protein, 7 aa, synthetic construct
ANYKRET

SEQ ID NO: 566 - protein, 7 aa, synthetic construct
INMKRVP

SEQ ID NO: 567 - protein, 7 aa, synthetic construct
MNNARPY

SEQ ID NO: 568 - protein, 7 aa, synthetic construct
SNNVRHY

SEQ ID NO: 569 - protein, 7 aa, synthetic construct
DNARRTL

SEQ ID NO: 570 - protein, 7 aa, synthetic construct
INPRKDN

SEQ ID NO: 571 - protein, 7 aa, synthetic construct
MNQIRAA

SEQ ID NO: 572 - protein, 7 aa, synthetic construct
SNPTRQY

SEQ ID NO: 573 - protein, 7 aa, synthetic construct
DNAVRPW

SEQ ID NO: 574 - protein, 7 aa, synthetic construct
INQIRAA

SEQ ID NO: 575 - protein, 7 aa, synthetic construct
MNQSRGW

SEQ ID NO: 576 - protein, 7 aa, synthetic construct
SNRQREL

SEQ ID NO: 577 - protein, 7 aa, synthetic construct
DNAVRSK

SEQ ID NO: 578 - protein, 7 aa, synthetic construct
INQIRQV

SEQ ID NO: 579 - protein, 7 aa, synthetic construct
MNRSRAE

SEQ ID NO: 580 - protein, 7 aa, synthetic construct
SNSHKGW

SEQ ID NO: 581 - protein, 7 aa, synthetic construct
DNEIRRW

SEQ ID NO: 582 - protein, 7 aa, synthetic construct
INSIRMT

SEQ ID NO: 583 - protein, 7 aa, synthetic construct
MNRTRFE

SEQ ID NO: 584 - protein, 7 aa, synthetic construct
SNTGKSW

SEQ ID NO: 585 - protein, 7 aa, synthetic construct
DNERRPR

SEQ ID NO: 586 - protein, 7 aa, synthetic construct
INSLRLV

SEQ ID NO: 587 - protein, 7 aa, synthetic construct
MNSPRSY

SEQ ID NO: 588 - protein, 7 aa, synthetic construct
SNTIKNL

SEQ ID NO: 589 - protein, 7 aa, synthetic construct
DNHIRSQ

SEQ ID NO: 590 - protein, 7 aa, synthetic construct
INSPRLF

SEQ ID NO: 591 - protein, 7 aa, synthetic construct
MNTIRSY

SEQ ID NO: 592 - protein, 7 aa, synthetic construct
SNTVRTI

SEQ ID NO: 593 - protein, 7 aa, synthetic construct
DNNPRKS

SEQ ID NO: 594 - protein, 7 aa, synthetic construct
INSPRVT

SEQ ID NO: 595 - protein, 7 aa, synthetic construct
MNTVRAW

SEQ ID NO: 596 - protein, 7 aa, synthetic construct
TNDGRSR

SEQ ID NO: 597 - protein, 7 aa, synthetic construct
DNPNKRQ

SEQ ID NO: 598 - protein, 7 aa, synthetic construct
INSQRTP

SEQ ID NO: 599 - protein, 7 aa, synthetic construct
MNTVRDY

SEQ ID NO: 600 - protein, 7 aa, synthetic construct
TNFGRTT

SEQ ID NO: 601 - protein, 7 aa, synthetic construct
DNVRRSN

SEQ ID NO: 602 - protein, 7 aa, synthetic construct
INSTRYP

SEQ ID NO: 603 - protein, 7 aa, synthetic construct
MNVLKKG

SEQ ID NO: 604 - protein, 7 aa, synthetic construct
TNHIRFT

SEQ ID NO: 605 - protein, 7 aa, synthetic construct
ENHLRNT

SEQ ID NO: 606 - protein, 7 aa, synthetic construct
INSYKGA

SEQ ID NO: 607 - protein, 7 aa, synthetic construct
NNALKPY

SEQ ID NO: 608 - protein, 7 aa, synthetic construct
TNHIRNL

SEQ ID NO: 609 - protein, 7 aa, synthetic construct
ENRSKNN

SEQ ID NO: 610 - protein, 7 aa, synthetic construct
INTSRSA

SEQ ID NO: 611 - protein, 7 aa, synthetic construct
NNFSRNG

SEQ ID NO: 612 - protein, 7 aa, synthetic construct
TNMIRNA

SEQ ID NO: 613 - protein, 7 aa, synthetic construct
ENSIRMY

SEQ ID NO: 614 - protein, 7 aa, synthetic construct
INTVRDR

SEQ ID NO: 615 - protein, 7 aa, synthetic construct
NNLNKYL

SEQ ID NO: 616 - protein, 7 aa, synthetic construct
TNMPRNS

SEQ ID NO: 617 - protein, 7 aa, synthetic construct
ENVVRSK

SEQ ID NO: 618 - protein, 7 aa, synthetic construct
INTVRSS

SEQ ID NO: 619 - protein, 7 aa, synthetic construct
NNMPRGG

SEQ ID NO: 620 - protein, 7 aa, synthetic construct
TNPARYG

SEQ ID NO: 621 - protein, 7 aa, synthetic construct
FNGGRMG

SEQ ID NO: 622 - protein, 7 aa, synthetic construct
KNAIKLG

SEQ ID NO: 623 - protein, 7 aa, synthetic construct
NNNVRFL

SEQ ID NO: 624 - protein, 7 aa, synthetic construct
TNPNRSS

SEQ ID NO: 625 - protein, 7 aa, synthetic construct
FNKTRGP

SEQ ID NO: 626 - protein, 7 aa, synthetic construct
KNLLRSE

SEQ ID NO: 627 - protein, 7 aa, synthetic construct
NNPLKRV

SEQ ID NO: 628 - protein, 7 aa, synthetic construct
TNPSRFA

SEQ ID NO: 629 - protein, 7 aa, synthetic construct
FNRERNN

SEQ ID NO: 630 - protein, 7 aa, synthetic construct
LNERKYT

SEQ ID NO: 631 - protein, 7 aa, synthetic construct
NNSARPL

SEQ ID NO: 632 - protein, 7 aa, synthetic construct
TNQLRGY

SEQ ID NO: 633 - protein, 7 aa, synthetic construct
FNTKRDF

SEQ ID NO: 634 - protein, 7 aa, synthetic construct
LNERRYQ

SEQ ID NO: 635 - protein, 7 aa, synthetic construct
NNSTRAG

SEQ ID NO: 636 - protein, 7 aa, synthetic construct
TNQLRRD

SEQ ID NO: 637 - protein, 7 aa, synthetic construct
FNTLKLG

SEQ ID NO: 638 - protein, 7 aa, synthetic construct
LNGGRSW

SEQ ID NO: 639 - protein, 7 aa, synthetic construct
NNSTRGG

SEQ ID NO: 640 - protein, 7 aa, synthetic construct
TNSGKWS

SEQ ID NO: 641 - protein, 7 aa, synthetic construct
FNTPRTA

SEQ ID NO: 642 - protein, 7 aa, synthetic construct
LNHIRLS

SEQ ID NO: 643 - protein, 7 aa, synthetic construct
NNTIRYS

SEQ ID NO: 644 - protein, 7 aa, synthetic construct
TNSTRFT

SEQ ID NO: 645 - protein, 7 aa, synthetic construct
GNARRET

SEQ ID NO: 646 - protein, 7 aa, synthetic construct
LNHIRSL

SEQ ID NO: 647 - protein, 7 aa, synthetic construct
NNVIRGF

SEQ ID NO: 648 - protein, 7 aa, synthetic construct
VNDLRTR

SEQ ID NO: 649 - protein, 7 aa, synthetic construct
GNAVKST

SEQ ID NO: 650 - protein, 7 aa, synthetic construct
LNHMRNT

SEQ ID NO: 651 - protein, 7 aa, synthetic construct
NNYPRNM

SEQ ID NO: 652 - protein, 7 aa, synthetic construct
VNGHRSN

SEQ ID NO: 653 - protein, 7 aa, synthetic construct
GNAWKNA

SEQ ID NO: 654 - protein, 7 aa, synthetic construct
LNLNRYS

SEQ ID NO: 655 - protein, 7 aa, synthetic construct
PNMPRYE

SEQ ID NO: 656 - protein, 7 aa, synthetic construct
VNHVRLT

SEQ ID NO: 657 - protein, 7 aa, synthetic construct
GNDIRVR

SEQ ID NO: 658 - protein, 7 aa, synthetic construct
LNMSKYA

SEQ ID NO: 659 - protein, 7 aa, synthetic construct
PNNLRNV

SEQ ID NO: 660 - protein, 7 aa, synthetic construct
VNMLRDP

SEQ ID NO: 661 - protein, 7 aa, synthetic construct
GNESRYM

SEQ ID NO: 662 - protein, 7 aa, synthetic construct
LNNIRNV

SEQ ID NO: 663 - protein, 7 aa, synthetic construct
PNNVRQY

SEQ ID NO: 664 - protein, 7 aa, synthetic construct
VNMPRSV

SEQ ID NO: 665 - protein, 7 aa, synthetic construct
GNEVRYY

SEQ ID NO: 666 - protein, 7 aa, synthetic construct
LNNIRQV

SEQ ID NO: 667 - protein, 7 aa, synthetic construct
PNRYKDV

SEQ ID NO: 668 - protein, 7 aa, synthetic construct
VNNMRYP

SEQ ID NO: 669 - protein, 7 aa, synthetic construct
GNGVKWI

SEQ ID NO: 670 - protein, 7 aa, synthetic construct
LNNLRSP

SEQ ID NO: 671 - protein, 7 aa, synthetic construct
PNSIRRD

SEQ ID NO: 672 - protein, 7 aa, synthetic construct
VNSPRTP

SEQ ID NO: 673 - protein, 7 aa, synthetic construct
GNKFKQA

SEQ ID NO: 674 - protein, 7 aa, synthetic construct
LNNTKTA

SEQ ID NO: 675 - protein, 7 aa, synthetic construct
PNSYKNI

SEQ ID NO: 676 - protein, 7 aa, synthetic construct
VNTIRNV

SEQ ID NO: 677 - protein, 7 aa, synthetic construct
GNLGRSS

SEQ ID NO: 678 - protein, 7 aa, synthetic construct
LNNVRMI

SEQ ID NO: 679 - protein, 7 aa, synthetic construct
PNTIRNV

SEQ ID NO: 680 - protein, 7 aa, synthetic construct
WNAPRNA

SEQ ID NO: 681 - protein, 7 aa, synthetic construct
GNMIRND

SEQ ID NO: 682 - protein, 7 aa, synthetic construct
LNNVRSP

SEQ ID NO: 683 - protein, 7 aa, synthetic construct
PNTLRYA

SEQ ID NO: 684 - protein, 7 aa, synthetic construct
WNENKRL

SEQ ID NO: 685 - protein, 7 aa, synthetic construct
GNMPRSN

SEQ ID NO: 686 - protein, 7 aa, synthetic construct
LNNVRSV

SEQ ID NO: 687 - protein, 7 aa, synthetic construct
QNNIKAW

SEQ ID NO: 688 - protein, 7 aa, synthetic construct
WNSPRTN

SEQ ID NO: 689 - protein, 7 aa, synthetic construct
GNNTKAV

SEQ ID NO: 690 - protein, 7 aa, synthetic construct
LNPRKIG

SEQ ID NO: 691 - protein, 7 aa, synthetic construct
QNNNRPA

SEQ ID NO: 692 - protein, 7 aa, synthetic construct
YNNFKGN

SEQ ID NO: 693 - protein, 7 aa, synthetic construct
GNNVKQF

SEQ ID NO: 694 - protein, 7 aa, synthetic construct
LNQYRAA

SEQ ID NO: 695 - protein, 7 aa, synthetic construct
QNRMRND

SEQ ID NO: 696 - protein, 7 aa, synthetic construct
YNRNKFE

SEQ ID NO: 697 - protein, 7 aa, synthetic construct
GNSARNI

SEQ ID NO: 698 - protein, 7 aa, synthetic construct
LNRERSA

SEQ ID NO: 699 - protein, 7 aa, synthetic construct
QNRQRDT

SEQ ID NO: 700 - protein, 7 aa, synthetic construct
YNSGRNT

SEQ ID NO: 701 - protein, 7 aa, synthetic construct
GNSIRAL

SEQ ID NO: 702 - protein, 7 aa, synthetic construct
LNRVRDH

SEQ ID NO: 703 - protein, 7 aa, synthetic construct
QNSLRYS

SEQ ID NO: 704 - protein, 7 aa, synthetic construct
YNTGRLV

SEQ ID NO: 705 - protein, 7 aa, synthetic construct
GNSIRDT

SEQ ID NO: 706 - protein, 7 aa, synthetic construct
LNRVRGD

SEQ ID NO: 707 - protein, 7 aa, synthetic construct
RNDTRHI

SEQ ID NO: 708 - protein, 7 aa, synthetic construct
GNSIRTI

SEQ ID NO: 709 - protein, 7 aa, synthetic construct
LNSARSI

SEQ ID NO: 710 - protein, 7 aa, synthetic construct
RNFERAN

SEQ ID NO: 711 - protein, 7 aa, synthetic construct
GNSSRNL

SEQ ID NO: 712 - protein, 7 aa, synthetic construct
LNSGRSA

SEQ ID NO: 713 - protein, 7 aa, synthetic construct
RNGFREL

SEQ ID NO: 714 - protein, 7 aa, synthetic construct
GNSVRGW

SEQ ID NO: 715 - protein, 7 aa, synthetic construct
LNSLRHP

SEQ ID NO: 716 - protein, 7 aa, synthetic construct
RNPAKTG

SEQ ID NO: 717 - protein, 7 aa, synthetic construct
GNTIRDI

SEQ ID NO: 718 - protein, 7 aa, synthetic construct
LNSVRHV

SEQ ID NO: 719 - protein, 7 aa, synthetic construct
RNQDRTT

SEQ ID NO: 720 - protein, 7 aa, synthetic construct
GNVIRSH

SEQ ID NO: 721 - protein, 7 aa, synthetic construct
LNTFKAA

SEQ ID NO: 722 - protein, 7 aa, synthetic construct
RNVDRST

SEQ ID NO: 723 - protein, 7 aa, synthetic construct
GNVPRVF

SEQ ID NO: 724 - protein, 7 aa, synthetic construct
LNTIRST

SEQ ID NO: 725 - protein, 7 aa, synthetic construct
RNYIKSD

SEQ ID NO: 726 - protein, 7 aa, synthetic construct
GNVTRST

SEQ ID NO: 727 - protein, 7 aa, synthetic construct
LNTLRTI

SEQ ID NO: 728 - protein, 7 aa, synthetic construct
SNDGRYY

SEQ ID NO: 729 - protein, 7 aa, synthetic construct
GNYGRAQ

SEQ ID NO: 730 - protein, 7 aa, synthetic construct
LNTSRSW

SEQ ID NO: 731 - protein, 7 aa, synthetic construct
SNHIRLA

SEQ ID NO: 732 - protein, 7 aa, synthetic construct
GNYSRMD

SEQ ID NO: 733 - protein, 7 aa, synthetic construct
LNTVRHI

SEQ ID NO: 734 - protein, 7 aa, synthetic construct
SNHIRTL

SEQ ID NO: 735 - protein, 7 aa, synthetic construct
HNDTKRY

SEQ ID NO: 736 - protein, 7 aa, synthetic construct
LNYNKGY

SEQ ID NO: 737 - protein, 7 aa, synthetic construct
SNHVRAI

SEQ ID NO: 738 - protein, 7 aa, synthetic construct
INAPRTA

SEQ ID NO: 739 - protein, 7 aa, synthetic construct
LNYTRPA

SEQ ID NO: 740 - protein, 7 aa, synthetic construct
SNHVRFM

SEQ ID NO: 741 - protein, 7 aa, synthetic construct
ANEVRRG

SEQ ID NO: 742 - protein, 7 aa, synthetic construct
LNHIRSL

SEQ ID NO: 743 - protein, 7 aa, synthetic construct
QNRMRND

SEQ ID NO: 744 - protein, 7 aa, synthetic construct
ANHIRSL

SEQ ID NO: 745 - protein, 7 aa, synthetic construct
LNHMRNT

SEQ ID NO: 746 - protein, 7 aa, synthetic construct
QNTARFM

SEQ ID NO: 747 - protein, 7 aa, synthetic construct
ANNNRNY

SEQ ID NO: 748 - protein, 7 aa, synthetic construct
LNNIRNV

SEQ ID NO: 749 - protein, 7 aa, synthetic construct
RNDTRHI

SEQ ID NO: 750 - protein, 7 aa, synthetic construct
ANYKRET

SEQ ID NO: 751 - protein, 7 aa, synthetic construct
LNNIRQV

SEQ ID NO: 752 - protein, 7 aa, synthetic construct
RNYIKSD

SEQ ID NO: 753 - protein, 7 aa, synthetic construct
CNGTKRE

SEQ ID NO: 754 - protein, 7 aa, synthetic construct
LNNVRMI

SEQ ID NO: 755 - protein, 7 aa, synthetic construct
SNHIRTL

SEQ ID NO: 756 - protein, 7 aa, synthetic construct
CNRSRDG

SEQ ID NO: 757 - protein, 7 aa, synthetic construct
LNNVRSV

SEQ ID NO: 758 - protein, 7 aa, synthetic construct
SNHVRAI

SEQ ID NO: 759 - protein, 7 aa, synthetic construct
DNARRTL

SEQ ID NO: 760 - protein, 7 aa, synthetic construct
LNPRKIG

SEQ ID NO: 761 - protein, 7 aa, synthetic construct
SNHVRTS

SEQ ID NO: 762 - protein, 7 aa, synthetic construct
DNAVRSK

SEQ ID NO: 763 - protein, 7 aa, synthetic construct
LNPVRNA

SEQ ID NO: 764 - protein, 7 aa, synthetic construct
SNLRRTE

SEQ ID NO: 765 - protein, 7 aa, synthetic construct
DNEIRRW

SEQ ID NO: 766 - protein, 7 aa, synthetic construct
LNQYRAA

SEQ ID NO: 767 - protein, 7 aa, synthetic construct
SNNVRHY

SEQ ID NO: 768 - protein, 7 aa, synthetic construct
DNHIRSQ

SEQ ID NO: 769 - protein, 7 aa, synthetic construct
LNRVRDH

SEQ ID NO: 770 - protein, 7 aa, synthetic construct
SNPTRQY

SEQ ID NO: 771 - protein, 7 aa, synthetic construct
ENHLRNT

SEQ ID NO: 772 - protein, 7 aa, synthetic construct
LNRVRGD

SEQ ID NO: 773 - protein, 7 aa, synthetic construct
SNRQREL

SEQ ID NO: 774 - protein, 7 aa, synthetic construct
ENPPRPR

SEQ ID NO: 775 - protein, 7 aa, synthetic construct
LNSARSI

SEQ ID NO: 776 - protein, 7 aa, synthetic construct
SNSIRFI

SEQ ID NO: 777 - protein, 7 aa, synthetic construct
ENRSKNN

SEQ ID NO: 778 - protein, 7 aa, synthetic construct
LNSGRSA

SEQ ID NO: 779 - protein, 7 aa, synthetic construct
SNTIKNL

SEQ ID NO: 780 - protein, 7 aa, synthetic construct
ENSIRMY

SEQ ID NO: 781 - protein, 7 aa, synthetic construct
LNSRREV

SEQ ID NO: 782 - protein, 7 aa, synthetic construct
SNTIRTI

SEQ ID NO: 783 - protein, 7 aa, synthetic construct
ENYGRGA

SEQ ID NO: 784 - protein, 7 aa, synthetic construct
LNSVRHV

SEQ ID NO: 785 - protein, 7 aa, synthetic construct
SNTVRTI

SEQ ID NO: 786 - protein, 7 aa, synthetic construct
GNAVKST

SEQ ID NO: 787 - protein, 7 aa, synthetic construct
LNTFKAA

SEQ ID NO: 788 - protein, 7 aa, synthetic construct
TNFGRTT

SEQ ID NO: 789 - protein, 7 aa, synthetic construct
GNDIRVR

SEQ ID NO: 790 - protein, 7 aa, synthetic construct
LNTIRST

SEQ ID NO: 791 - protein, 7 aa, synthetic construct
TNHIRFT

SEQ ID NO: 792 - protein, 7 aa, synthetic construct
GNDIRYY

SEQ ID NO: 793 - protein, 7 aa, synthetic construct
LNTIRTN

SEQ ID NO: 794 - protein, 7 aa, synthetic construct
TNHIRNL

SEQ ID NO: 795 - protein, 7 aa, synthetic construct
GNEVRYY

SEQ ID NO: 796 - protein, 7 aa, synthetic construct
LNTIRVV

SEQ ID NO: 797 - protein, 7 aa, synthetic construct
TNMIRNA

SEQ ID NO: 798 - protein, 7 aa, synthetic construct
GNGVKWI

SEQ ID NO: 799 - protein, 7 aa, synthetic construct
LNTVRDI

SEQ ID NO: 800 - protein, 7 aa, synthetic construct
TNSTRFT

SEQ ID NO: 801 - protein, 7 aa, synthetic construct
GNMIRND

SEQ ID NO: 802 - protein, 7 aa, synthetic construct
LNTVRHI

SEQ ID NO: 803 - protein, 7 aa, synthetic construct
TNVLRGF

SEQ ID NO: 804 - protein, 7 aa, synthetic construct
GNNTKAV

SEQ ID NO: 805 - protein, 7 aa, synthetic construct
LNYTRSS

SEQ ID NO: 806 - protein, 7 aa, synthetic construct
VNHIRLQ

SEQ ID NO: 807 - protein, 7 aa, synthetic construct
GNNVKQF

SEQ ID NO: 808 - protein, 7 aa, synthetic construct
MNDVRSV

SEQ ID NO: 809 - protein, 7 aa, synthetic construct
VNHVRLT

SEQ ID NO: 810 - protein, 7 aa, synthetic construct
GNSARNI

SEQ ID NO: 811 - protein, 7 aa, synthetic construct
MNHIRTT

SEQ ID NO: 812 - protein, 7 aa, synthetic construct
VNNERSK

SEQ ID NO: 813 - protein, 7 aa, synthetic construct
GNSIRAL

SEQ ID NO: 814 - protein, 7 aa, synthetic construct
MNHPRMI

SEQ ID NO: 815 - protein, 7 aa, synthetic construct
VNSPRSI

SEQ ID NO: 816 - protein, 7 aa, synthetic construct
GNSIRDT

SEQ ID NO: 817 - protein, 7 aa, synthetic construct
MNNARPY

SEQ ID NO: 818 - protein, 7 aa, synthetic construct
VNTIRNV

SEQ ID NO: 819 - protein, 7 aa, synthetic construct
GNSIRTI

SEQ ID NO: 820 - protein, 7 aa, synthetic construct
MNQSRGW

SEQ ID NO: 821 - protein, 7 aa, synthetic construct
VNTIRSV

SEQ ID NO: 822 - protein, 7 aa, synthetic construct
GNSMRHM

SEQ ID NO: 823 - protein, 7 aa, synthetic construct
MNRTRFE

SEQ ID NO: 824 - protein, 7 aa, synthetic construct
WNSPRTN

SEQ ID NO: 825 - protein, 7 aa, synthetic construct
GNSSRNL

SEQ ID NO: 826 - protein, 7 aa, synthetic construct
MNSPRSL

SEQ ID NO: 827 - protein, 7 aa, synthetic construct
YNRNKFE

SEQ ID NO: 828 - protein, 7 aa, synthetic construct
GNSVRGW

SEQ ID NO: 829 - protein, 7 aa, synthetic construct
MNTIRSY

SEQ ID NO: 830 - protein, 7 aa, synthetic construct
YNSGRNT

SEQ ID NO: 831 - protein, 7 aa, synthetic construct
GNTIRDI

SEQ ID NO: 832 - protein, 7 aa, synthetic construct
MNTVRAW

SEQ ID NO: 833 - protein, 7 aa, synthetic construct
YNTGRLV

SEQ ID NO: 834 - protein, 7 aa, synthetic construct
GNVIRSH

SEQ ID NO: 835 - protein, 7 aa, synthetic construct
MNTVRDY

SEQ ID NO: 836 - protein, 7 aa, synthetic construct
GNVMRTT

SEQ ID NO: 837 - protein, 7 aa, synthetic construct
NNASKGW

SEQ ID NO: 838 - protein, 7 aa, synthetic construct
GNVTRST

SEQ ID NO: 839 - protein, 7 aa, synthetic construct
NNFSRNG

SEQ ID NO: 840 - protein, 7 aa, synthetic construct
GNYGRAQ

SEQ ID NO: 841 - protein, 7 aa, synthetic construct
NNNVRFL

SEQ ID NO: 842 - protein, 7 aa, synthetic construct
HNMSRIA

SEQ ID NO: 843 - protein, 7 aa, synthetic construct
NNPVRIP

SEQ ID NO: 844 - protein, 7 aa, synthetic construct
INHLKSA

SEQ ID NO: 845 - protein, 7 aa, synthetic construct
NNSARPL

SEQ ID NO: 846 - protein, 7 aa, synthetic construct
INHMRGA

SEQ ID NO: 847 - protein, 7 aa, synthetic construct
NNSTRAG

SEQ ID NO: 848 - protein, 7 aa, synthetic construct
INMPRDT

SEQ ID NO: 849 - protein, 7 aa, synthetic construct
NNSTRGG

SEQ ID NO: 850 - protein, 7 aa, synthetic construct
INNARIP

SEQ ID NO: 851 - protein, 7 aa, synthetic construct
NNTIRYS

SEQ ID NO: 852 - protein, 7 aa, synthetic construct
INQIRQV

SEQ ID NO: 853 - protein, 7 aa, synthetic construct
NNVIRGF

SEQ ID NO: 854 - protein, 7 aa, synthetic construct
INSPRLF

SEQ ID NO: 855 - protein, 7 aa, synthetic construct
PNHVRIA

SEQ ID NO: 856 - protein, 7 aa, synthetic construct
INSPRVS

SEQ ID NO: 857 - protein, 7 aa, synthetic construct
PNNLRNV

SEQ ID NO: 858 - protein, 7 aa, synthetic construct
INSPRVT

SEQ ID NO: 859 - protein, 7 aa, synthetic construct
PNNVRQY

SEQ ID NO: 860 - protein, 7 aa, synthetic construct
INTSRSA

SEQ ID NO: 861 - protein, 7 aa, synthetic construct
PNSIRFS

SEQ ID NO: 862 - protein, 7 aa, synthetic construct
INTVRDR

SEQ ID NO: 863 - protein, 7 aa, synthetic construct
PNSIRRD

SEQ ID NO: 864 - protein, 7 aa, synthetic construct
INTVRSS

SEQ ID NO: 865 - protein, 7 aa, synthetic construct
PNTIRNV

SEQ ID NO: 866 - protein, 7 aa, synthetic construct
KNAIKLG

SEQ ID NO: 867 - protein, 7 aa, synthetic construct
PNTLRYA

SEQ ID NO: 868 - protein, 7 aa, synthetic construct
KNNLREY

SEQ ID NO: 869 - protein, 7 aa, synthetic construct
QNLGRYV

SEQ ID NO: 870 - protein, 7 aa, synthetic construct
LNEVKLY

SEQ ID NO: 871 - protein, 7 aa, synthetic construct
QNNIKAW

SEQ ID NO: 872 - protein, 7 aa, synthetic construct
ANGTRGH

SEQ ID NO: 873 - protein, 7 aa, synthetic construct
INHVRCV

SEQ ID NO: 874 - protein, 7 aa, synthetic construct
LNYNKGY

SEQ ID NO: 875 - protein, 7 aa, synthetic construct
SNHIRLA

SEQ ID NO: 876 - protein, 7 aa, synthetic construct
ANHIRSL

SEQ ID NO: 877 - protein, 7 aa, synthetic construct
INIIRVP

SEQ ID NO: 878 - protein, 7 aa, synthetic construct
LNYTRPA

SEQ ID NO: 879 - protein, 7 aa, synthetic construct
SNHIRTL

SEQ ID NO: 880 - protein, 7 aa, synthetic construct
ANKNKPV

SEQ ID NO: 881 - protein, 7 aa, synthetic construct
INITRNH

SEQ ID NO: 882 - protein, 7 aa, synthetic construct
LNYTRSS

SEQ ID NO: 883 - protein, 7 aa, synthetic construct
SNHRRME

SEQ ID NO: 884 - protein, 7 aa, synthetic construct
ANRDRYT

SEQ ID NO: 885 - protein, 7 aa, synthetic construct
INMKRVP

SEQ ID NO: 886 - protein, 7 aa, synthetic construct
MNHIRTT

SEQ ID NO: 887 - protein, 7 aa, synthetic construct
SNHVRAI

SEQ ID NO: 888 - protein, 7 aa, synthetic construct
ANSQRHS

SEQ ID NO: 889 - protein, 7 aa, synthetic construct
INPKRTA

SEQ ID NO: 890 - protein, 7 aa, synthetic construct
MNHPRMI

SEQ ID NO: 891 - protein, 7 aa, synthetic construct
SNHVRFM

SEQ ID NO: 892 - protein, 7 aa, synthetic construct
ANYKRET

SEQ ID NO: 893 - protein, 7 aa, synthetic construct
INQIRAA

SEQ ID NO: 894 - protein, 7 aa, synthetic construct
MNHYRPQ

SEQ ID NO: 895 - protein, 7 aa, synthetic construct
SNHVRTS

SEQ ID NO: 896 - protein, 7 aa, synthetic construct
CNKPKDN

SEQ ID NO: 897 - protein, 7 aa, synthetic construct
INSIRMT

SEQ ID NO: 898 - protein, 7 aa, synthetic construct
INSIRMT

SEQ ID NO: 899 - protein, 7 aa, synthetic construct
SNKNRWE

SEQ ID NO: 900 - protein, 7 aa, synthetic construct
DNARRVT

SEQ ID NO: 901 - protein, 7 aa, synthetic construct
INSKKNA

SEQ ID NO: 902 - protein, 7 aa, synthetic construct
MNMPRTS

SEQ ID NO: 903 - protein, 7 aa, synthetic construct
SNLRRTE

SEQ ID NO: 904 - protein, 7 aa, synthetic construct
DNAVRSK

SEQ ID NO: 905 - protein, 7 aa, synthetic construct
INSPRVT

SEQ ID NO: 906 - protein, 7 aa, synthetic construct
MNNARPY

SEQ ID NO: 907 - protein, 7 aa, synthetic construct
SNNGRYM

SEQ ID NO: 908 - protein, 7 aa, synthetic construct
DNEIRRW

SEQ ID NO: 909 - protein, 7 aa, synthetic construct
INSTRYP

SEQ ID NO: 910 - protein, 7 aa, synthetic construct
MNNSRMP

SEQ ID NO: 911 - protein, 7 aa, synthetic construct
SNNVRHY

SEQ ID NO: 912 - protein, 7 aa, synthetic construct
DNERRPR

SEQ ID NO: 913 - protein, 7 aa, synthetic construct
INSVRIP

SEQ ID NO: 914 - protein, 7 aa, synthetic construct
MNQSRGW

SEQ ID NO: 915 - protein, 7 aa, synthetic construct
SNRDRNS

SEQ ID NO: 916 - protein, 7 aa, synthetic construct
DNHIRSQ

SEQ ID NO: 917 - protein, 7 aa, synthetic construct
INSYKGA

SEQ ID NO: 918 - protein, 7 aa, synthetic construct
MNRSRAE

SEQ ID NO: 919 - protein, 7 aa, synthetic construct
SNRNRDY

SEQ ID NO: 920 - protein, 7 aa, synthetic construct
DNNPRKS

SEQ ID NO: 921 - protein, 7 aa, synthetic construct
INTVRSS

SEQ ID NO: 922 - protein, 7 aa, synthetic construct
MNRTRFE

SEQ ID NO: 923 - protein, 7 aa, synthetic construct
SNSHKGW

SEQ ID NO: 924 - protein, 7 aa, synthetic construct
DNVRRSN

SEQ ID NO: 925 - protein, 7 aa, synthetic construct
INYQKPA

SEQ ID NO: 926 - protein, 7 aa, synthetic construct
MNSPRSL

SEQ ID NO: 927 - protein, 7 aa, synthetic construct
SNSIRFI

SEQ ID NO: 928 - protein, 7 aa, synthetic construct
ENDKKNK

SEQ ID NO: 929 - protein, 7 aa, synthetic construct
INYVKHH

SEQ ID NO: 930 - protein, 7 aa, synthetic construct
MNSPRSY

SEQ ID NO: 931 - protein, 7 aa, synthetic construct
SNTGKSW

SEQ ID NO: 932 - protein, 7 aa, synthetic construct
ENMPRPT

SEQ ID NO: 933 - protein, 7 aa, synthetic construct
KNAIKLG

SEQ ID NO: 934 - protein, 7 aa, synthetic construct
MNTIRSY

SEQ ID NO: 935 - protein, 7 aa, synthetic construct
SNTIKNL

SEQ ID NO: 936 - protein, 7 aa, synthetic construct
ENNGRRN

SEQ ID NO: 937 - protein, 7 aa, synthetic construct
KNALKSS

SEQ ID NO: 938 - protein, 7 aa, synthetic construct
MNTVRAW

SEQ ID NO: 939 - protein, 7 aa, synthetic construct
SNTIRTI

SEQ ID NO: 940 - protein, 7 aa, synthetic construct
ENNRRQM

SEQ ID NO: 941 - protein, 7 aa, synthetic construct
KNDERHR

SEQ ID NO: 942 - protein, 7 aa, synthetic construct
MNVLKKG

SEQ ID NO: 943 - protein, 7 aa, synthetic construct
TNERKYL

SEQ ID NO: 944 - protein, 7 aa, synthetic construct
ENRQKYT

SEQ ID NO: 945 - protein, 7 aa, synthetic construct
KNGGKPN

SEQ ID NO: 946 - protein, 7 aa, synthetic construct
NNFSRNG

SEQ ID NO: 947 - protein, 7 aa, synthetic construct
TNFGRTT

SEQ ID NO: 948 - protein, 7 aa, synthetic construct
ENRSKNN

SEQ ID NO: 949 - protein, 7 aa, synthetic construct
KNHNKPG

SEQ ID NO: 950 - protein, 7 aa, synthetic construct
NNILRVG

SEQ ID NO: 951 - protein, 7 aa, synthetic construct
TNHIRFT

SEQ ID NO: 952 - protein, 7 aa, synthetic construct
ENSLRHR

SEQ ID NO: 953 - protein, 7 aa, synthetic construct
KNLMRID

SEQ ID NO: 954 - protein, 7 aa, synthetic construct
NNMPRGG

SEQ ID NO: 955 - protein, 7 aa, synthetic construct
TNHIRHL

SEQ ID NO: 956 - protein, 7 aa, synthetic construct
FNGGRMG

SEQ ID NO: 957 - protein, 7 aa, synthetic construct
KNPIKSS

SEQ ID NO: 958 - protein, 7 aa, synthetic construct
NNPLKRV

SEQ ID NO: 959 - protein, 7 aa, synthetic construct
TNHIRHP

SEQ ID NO: 960 - protein, 7 aa, synthetic construct
FNKTRGP

SEQ ID NO: 961 - protein, 7 aa, synthetic construct
LNDSRRP

SEQ ID NO: 962 - protein, 7 aa, synthetic construct
NNPVRIP

SEQ ID NO: 963 - protein, 7 aa, synthetic construct
TNHIRNL

SEQ ID NO: 964 - protein, 7 aa, synthetic construct
FNRERGP

SEQ ID NO: 965 - protein, 7 aa, synthetic construct
LNERRYQ

SEQ ID NO: 966 - protein, 7 aa, synthetic construct
NNSTRAG

SEQ ID NO: 967 - protein, 7 aa, synthetic construct
TNKTRPA

SEQ ID NO: 968 - protein, 7 aa, synthetic construct
FNRERNN

SEQ ID NO: 969 - protein, 7 aa, synthetic construct
LNHARYP

SEQ ID NO: 970 - protein, 7 aa, synthetic construct
NNSTRGG

SEQ ID NO: 971 - protein, 7 aa, synthetic construct
TNMPRNS

SEQ ID NO: 972 - protein, 7 aa, synthetic construct
FNTKRDF

SEQ ID NO: 973 - protein, 7 aa, synthetic construct
LNHGRTA

SEQ ID NO: 974 - protein, 7 aa, synthetic construct
NNTIRYS

SEQ ID NO: 975 - protein, 7 aa, synthetic construct
TNPARYG

SEQ ID NO: 976 - protein, 7 aa, synthetic construct
FNTPRTA

SEQ ID NO: 977 - protein, 7 aa, synthetic construct
LNHIRLS

SEQ ID NO: 978 - protein, 7 aa, synthetic construct
NNTYRSS

SEQ ID NO: 979 - protein, 7 aa, synthetic construct
TNQLRRD

SEQ ID NO: 980 - protein, 7 aa, synthetic construct
GNAVKST

SEQ ID NO: 981 - protein, 7 aa, synthetic construct
LNHIRSL

SEQ ID NO: 982 - protein, 7 aa, synthetic construct
NNVIRGF

SEQ ID NO: 983 - protein, 7 aa, synthetic construct
TNSGKWS

SEQ ID NO: 984 - protein, 7 aa, synthetic construct
GNAWKNA

SEQ ID NO: 985 - protein, 7 aa, synthetic construct
LNHIRTS

SEQ ID NO: 986 - protein, 7 aa, synthetic construct
PNEYKAR

SEQ ID NO: 987 - protein, 7 aa, synthetic construct
TNSIRLP

SEQ ID NO: 988 - protein, 7 aa, synthetic construct
GNDIRVR

SEQ ID NO: 989 - protein, 7 aa, synthetic construct
LNHMRNT

SEQ ID NO: 990 - protein, 7 aa, synthetic construct
PNHPRHL

SEQ ID NO: 991 - protein, 7 aa, synthetic construct
TNSLRHP

SEQ ID NO: 992 - protein, 7 aa, synthetic construct
GNKFKQA

SEQ ID NO: 993 - protein, 7 aa, synthetic construct
LNIRRGE

SEQ ID NO: 994 - protein, 7 aa, synthetic construct
PNMPRYE

SEQ ID NO: 995 - protein, 7 aa, synthetic construct
TNSLRSI

SEQ ID NO: 996 - protein, 7 aa, synthetic construct
GNLGRSS

SEQ ID NO: 997 - protein, 7 aa, synthetic construct
LNKLRGP

SEQ ID NO: 998 - protein, 7 aa, synthetic construct
PNNLRTP

SEQ ID NO: 999 - protein, 7 aa, synthetic construct
TNSTRFT

SEQ ID NO: 1000 - protein, 7 aa, synthetic construct
GNLQRYQ

SEQ ID NO: 1001 - protein, 7 aa, synthetic construct
LNLNRYS

SEQ ID NO: 1002 - protein, 7 aa, synthetic construct
PNNVRQY

SEQ ID NO: 1003 - protein, 7 aa, synthetic construct
VNEVRMA

SEQ ID NO: 1004 - protein, 7 aa, synthetic construct
GNMIRND

SEQ ID NO: 1005 - protein, 7 aa, synthetic construct
LNMPRTN

SEQ ID NO: 1006 - protein, 7 aa, synthetic construct
PNRYKDV

SEQ ID NO: 1007 - protein, 7 aa, synthetic construct
VNHIRLQ

SEQ ID NO: 1008 - protein, 7 aa, synthetic construct
GNNCKAT

SEQ ID NO: 1009 - protein, 7 aa, synthetic construct
LNMSKYA

SEQ ID NO: 1010 - protein, 7 aa, synthetic construct
PNSLRAI

SEQ ID NO: 1011 - protein, 7 aa, synthetic construct
VNHVRLT

SEQ ID NO: 1012 - protein, 7 aa, synthetic construct
GNNTKAV

SEQ ID NO: 1013 - protein, 7 aa, synthetic construct
LNNIRNV

SEQ ID NO: 1014 - protein, 7 aa, synthetic construct
PNSLRER

SEQ ID NO: 1015 - protein, 7 aa, synthetic construct
VNLHRSG

SEQ ID NO: 1016 - protein, 7 aa, synthetic construct
GNNVKQF

SEQ ID NO: 1017 - protein, 7 aa, synthetic construct
LNNIRQV

SEQ ID NO: 1018 - protein, 7 aa, synthetic construct
PNSMRQV

SEQ ID NO: 1019 - protein, 7 aa, synthetic construct
VNNLRTL

SEQ ID NO: 1020 - protein, 7 aa, synthetic construct
GNSARNI

SEQ ID NO: 1021 - protein, 7 aa, synthetic construct
LNNVRSP

SEQ ID NO: 1022 - protein, 7 aa, synthetic construct
PNTIRNV

SEQ ID NO: 1023 - protein, 7 aa, synthetic construct
VNNMRYP

SEQ ID NO: 1024 - protein, 7 aa, synthetic construct
GNSIRAL

SEQ ID NO: 1025 - protein, 7 aa, synthetic construct
LNNVRSV

SEQ ID NO: 1026 - protein, 7 aa, synthetic construct
PNTLRFA

SEQ ID NO: 1027 - protein, 7 aa, synthetic construct
VNPNRSG

SEQ ID NO: 1028 - protein, 7 aa, synthetic construct
GNSIRDT

SEQ ID NO: 1029 - protein, 7 aa, synthetic construct
LNPRKIG

SEQ ID NO: 1030 - protein, 7 aa, synthetic construct
QNKHREM

SEQ ID NO: 1031 - protein, 7 aa, synthetic construct
VNSLRQY

SEQ ID NO: 1032 - protein, 7 aa, synthetic construct
GNSIRTI

SEQ ID NO: 1033 - protein, 7 aa, synthetic construct
LNPVRNA

SEQ ID NO: 1034 - protein, 7 aa, synthetic construct
QNNIKAW

SEQ ID NO: 1035 - protein, 7 aa, synthetic construct
VNTGKGW

SEQ ID NO: 1036 - protein, 7 aa, synthetic construct
GNSLRAY

SEQ ID NO: 1037 - protein, 7 aa, synthetic construct
LNQYRAA

SEQ ID NO: 1038 - protein, 7 aa, synthetic construct
QNNLKYL

SEQ ID NO: 1039 - protein, 7 aa, synthetic construct
VNTIRNV

SEQ ID NO: 1040 - protein, 7 aa, synthetic construct
GNSSRNL

SEQ ID NO: 1041 - protein, 7 aa, synthetic construct
LNRERSA

SEQ ID NO: 1042 - protein, 7 aa, synthetic construct
QNRMRND

SEQ ID NO: 1043 - protein, 7 aa, synthetic construct
VNTPRHS

SEQ ID NO: 1044 - protein, 7 aa, synthetic construct
GNSVRGW

SEQ ID NO: 1045 - protein, 7 aa, synthetic construct
LNRVRDH

SEQ ID NO: 1046 - protein, 7 aa, synthetic construct
QNRQRDT

SEQ ID NO: 1047 - protein, 7 aa, synthetic construct
WNAPRNA

SEQ ID NO: 1048 - protein, 7 aa, synthetic construct
GNTIRDI

SEQ ID NO: 1049 - protein, 7 aa, synthetic construct
LNRVRGD

SEQ ID NO: 1050 - protein, 7 aa, synthetic construct
QNSLRYS

SEQ ID NO: 1051 - protein, 7 aa, synthetic construct
WNEYRSS

SEQ ID NO: 1052 - protein, 7 aa, synthetic construct
GNTYRDY

SEQ ID NO: 1053 - protein, 7 aa, synthetic construct
LNSARSI

SEQ ID NO: 1054 - protein, 7 aa, synthetic construct
QNTARFM

SEQ ID NO: 1055 - protein, 7 aa, synthetic construct
WNHPRAA

SEQ ID NO: 1056 - protein, 7 aa, synthetic construct
GNVIRSH

SEQ ID NO: 1057 - protein, 7 aa, synthetic construct
LNSLRHP

SEQ ID NO: 1058 - protein, 7 aa, synthetic construct
RNDGRVA

SEQ ID NO: 1059 - protein, 7 aa, synthetic construct
WNNFRPS

SEQ ID NO: 1060 - protein, 7 aa, synthetic construct
GNVPRVF

SEQ ID NO: 1061 - protein, 7 aa, synthetic construct
LNSPRDR

SEQ ID NO: 1062 - protein, 7 aa, synthetic construct
RNEVRFA

SEQ ID NO: 1063 - protein, 7 aa, synthetic construct
WNPGRAG

SEQ ID NO: 1064 - protein, 7 aa, synthetic construct
GNVTRST

SEQ ID NO: 1065 - protein, 7 aa, synthetic construct
LNSPRFV

SEQ ID NO: 1066 - protein, 7 aa, synthetic construct
LNSPRFV

SEQ ID NO: 1067 - protein, 7 aa, synthetic construct
WNSNRFE

SEQ ID NO: 1068 - protein, 7 aa, synthetic construct
GNYGRAQ

SEQ ID NO: 1069 - protein, 7 aa, synthetic construct
LNSPRIT

SEQ ID NO: 1070 - protein, 7 aa, synthetic construct
RNFNKND

SEQ ID NO: 1071 - protein, 7 aa, synthetic construct
WNSPRNT

SEQ ID NO: 1072 - protein, 7 aa, synthetic construct
HNDTKRY

SEQ ID NO: 1073 - protein, 7 aa, synthetic construct
LNSRREV

SEQ ID NO: 1074 - protein, 7 aa, synthetic construct
RNMAKAP

SEQ ID NO: 1075 - protein, 7 aa, synthetic construct
WNSPRTN

SEQ ID NO: 1076 - protein, 7 aa, synthetic construct
HNEERKR

SEQ ID NO: 1077 - protein, 7 aa, synthetic construct
LNSVRHV

SEQ ID NO: 1078 - protein, 7 aa, synthetic construct
RNNGRPQ

SEQ ID NO: 1079 - protein, 7 aa, synthetic construct
YNAHRGA

SEQ ID NO: 1080 - protein, 7 aa, synthetic construct
INAPRTA

SEQ ID NO: 1081 - protein, 7 aa, synthetic construct
LNTFKAA

SEQ ID NO: 1082 - protein, 7 aa, synthetic construct
RNNPKPL

SEQ ID NO: 1083 - protein, 7 aa, synthetic construct
YNMPRGA

SEQ ID NO: 1084 - protein, 7 aa, synthetic construct
INDLRTP

SEQ ID NO: 1085 - protein, 7 aa, synthetic construct
LNTIRST

SEQ ID NO: 1086 - protein, 7 aa, synthetic construct
RNPAKTG

SEQ ID NO: 1087 - protein, 7 aa, synthetic construct
YNNFKGN

SEQ ID NO: 1088 - protein, 7 aa, synthetic construct
INHIRML

SEQ ID NO: 1089 - protein, 7 aa, synthetic construct
LNTPRST

SEQ ID NO: 1090 - protein, 7 aa, synthetic construct
RNVDRST

SEQ ID NO: 1091 - protein, 7 aa, synthetic construct
YNQMRNT

SEQ ID NO: 1092 - protein, 7 aa, synthetic construct
INHLKSA

SEQ ID NO: 1093 - protein, 7 aa, synthetic construct
LNTSRSW

SEQ ID NO: 1094 - protein, 7 aa, synthetic construct
RNYIKSD

SEQ ID NO: 1095 - protein, 7 aa, synthetic construct
YNRNKFE

SEQ ID NO: 1096 - protein, 7 aa, synthetic construct
INHMRGA

SEQ ID NO: 1097 - protein, 7 aa, synthetic construct
LNTVRHI

SEQ ID NO: 1098 - protein, 7 aa, synthetic construct
SNFIRSA

SEQ ID NO: 1099 - protein, 7 aa, synthetic construct
YNTGRLV

SEQ ID NO: 1100 - protein, 7 aa, synthetic construct
TALKPFL

SEQ ID NO: 1101 - protein, 7 aa, synthetic construct
TTLKPFL

SEQ ID NO: 1102 - protein, 9 aa, synthetic construct
DGTLAVPFK

SEQ ID NO: 1103 - protein, 7 aa, synthetic construct
TLAVPFK

SEQ ID NO: 1104 - protein, 7 aa, synthetic construct
NRGTEWD

SEQ ID NO: 1105 - protein, 7 aa, synthetic construct
DSPAHPS

SEQ ID NO: 1106 - DNA, 22 bp, synthetic construct
  source 1..22: mol_type = other DNA
gtgcatcagccgcgtctactcc

SEQ ID NO: 1107 - DNA, 27 bp, synthetic construct
  source 1..27: mol_type = other DNA
ggccgtaacctgaatctcttccctttg

SEQ ID NO: 1108 - DNA, 21 bp, synthetic construct
  source 1..21: mol_type = other DNA
ggaacccctagtgatggagtt

SEQ ID NO: 1109 - DNA, 16 bp, synthetic construct
  source 1..16: mol_type = other DNA
cggcctcagtgagcga

SEQ ID NO: 1110 - DNA, 21 bp, synthetic construct
  source 1..21: mol_type = other DNA
  misc_feature <1: note = HEX dye
  misc_feature 9..10: note = ZEN internal quencher
  misc_feature >21: note = Iowa Black fluorescent quencher
cactccctctctgcgcgctcg

SEQ ID NO: 1111 - DNA, 22 bp, synthetic construct
  source 1..22: mol_type = other DNA
cggactcagactatcagctccc

SEQ ID NO: 1112 - DNA, 83 bp, synthetic construct
  source 1..83: mol_type = other DNA
gtattccttggttttgaacccaaccggtctgcgcctgtgcmnnmnnmnnmnnmnnmnnmnnttgggcactctggtggtttgtg

SEQ ID NO: 1113 - DNA, 61 bp, synthetic construct
  source 1..61: mol_type = other DNA
ctttccctacacgacgctcttccgatctnccaacgaagaagaaattaaaactactaacccg

SEQ ID NO: 1114 - DNA, 53 bp, synthetic construct
  source 1..53: mol_type = other DNA
ggagttcagacgtgtgctcttccgatctcatctctgtcctgccaaaccatacc

SEQ ID NO: 1115 - DNA, 21 bp, synthetic construct
  source 1..21: mol_type = other DNA
aacaactcaacccgcggcggc

(19) United States
(12) Patent Application Publication
(10) Pub. No.: US 2024/0325568 A1
(43) Pub. Date: Oct. 3, 2024

(54) ENGINEERED TARGETING COMPOSITIONS FOR ENDOTHELIAL CELLS OF THE CENTRAL NERVOUS SYSTEM VASCULATURE AND METHODS OF USE THEREOF

(71) Applicant: The Broad Institute, Inc., Cambridge, MA (US)

(72) Inventors: Benjamin Deverman, Cambridge, MA (US); Qin Huang, Cambridge, MA (US); Ken Chan, Cambridge, MA (US)

(21) Appl. No.: 18/580,716

(22) PCT Filed: Jul. 20, 2022
(86) PCT No.: PCT/US2022/073968

§ 371 Date: Jan. 19, 2024

Related U.S. Application Data
(60) Provisional application No. 63/223,855, filed on Jul. 20, 2021.
(60) Provisional application No. 63/305,925, filed on Feb. 2, 2022.

(51) Int. Cl. A61K 48/00; A61K 31/7105; A61K 38/46; A61K 47/64; C12N 15/86

(52) U.S. Cl. CPC A61K 48/0033; A61K 31/7105; A61K 38/465; A61K 47/64; C12N 15/86; C12N 2750/14122; C12N 2750/14143; C12N 2750/14145

(57) ABSTRACT

Highly selective targeting moieties and compositions comprising the targeting moieties are described herein to efficiently transduce endothelial cell of the central nervous system vasculature. Embodiments include use and delivery of the targeting moieties and compositions to selectively direct delivery of cargo.

### CROSS-REFERENCE TO RELATED APPLICATIONS

This application claims the benefit of and priority to U.S. Provisional Patent Application No. 63/223,855 filed on Jul. 20, 2021, and U.S. Provisional Patent Application No. 63/305,925 filed on Feb. 2, 2022, the contents of which are incorporated by reference herein in its entirety.

### STATEMENT REGARDING FEDERALLY SPONSORED RESEARCH

This invention was made with government support under Grant No. NS 111689 and Grant No. MH120096 awarded by the National Institutes of Health. The government has certain rights in the invention.

### SEQUENCE LISTING

The contents of the electronic sequence listing (BROD-5415WP_ST26.xml, size is 965,380 bytes and it was created on Jul. 19, 2022) is herein incorporated by reference in its entirety.

### TECHNICAL FIELD

The subject matter disclosed herein is generally directed to non-naturally occurring or engineered adeno-associated virus (AAV) vectors with improved transduction properties. Further, the non-naturally occurring or engineered AAV vectors are designed to target, i.e., be delivered to the central nervous system and, specifically, the endothelial cells of the central nervous system vasculature.

### BACKGROUND

Recent strides in AAV development have produced engineered capsids capable of transducing well-defined cellular populations in the central nervous system (CNS) far more efficiently than their naturally occurring counterparts (Deverman, B. E. et al. (2016), Nat Biotechnol 34, 204-209; Körbelin, J. et al. (2016), EABO Mol Med 8, 609-625; Tervo, D. G. R. et al. (2016), Neuron 92, 372-382; Chan, K. Y. et al. (2017), Nat Neurosci 20, 1172-1179; Hanlon, K. S. et al. (2019), Mol Ther Methods Clin Dev 15, 320-332; Kumar, S. R. et al. (2020), Nat Methods 17, 541-550; Nonnenmacher, M. et al. (2020), Mol Ther Methods Clin Dev 20, 366-378). Leveraged as a rapid and flexible in vivo gene transfer platform, these engineered vectors are poised to act as a transformative catalyst for basic research when used in conjunction with—or as a substitute for—existing mouse genetics tools. However, capsid development has predominantly focused on vectors designed to transduce neurons or astrocytes. By comparison, relatively few vectors have been described that specifically target other cellular populations within the CNS, despite emerging appreciation that a vast repertoire of non-neuronal cell types are critical for nervous system function.

Among these, CNS endothelial cells—highly specialized cells that line the luminal face of blood vessels—have been shown to orchestrate a number of key physiological processes. Moreover, their dysfunction is increasingly appreciated to contribute to a wide range of neurodegenerative and neurological diseases (Sweeney, M. D., et al. (2018), Nat Neurosci 21, 1318-1331; Mastorakos, P. et al. (2019), Sci Immunol 4, eaav0492). However, a mismatch between the expanding functions ascribed to endothelial cells and the relatively limited tools available to study them in vivo is a major obstacle to research progress. While CNS endothelial cells are often regarded as a relatively homogenous entity, recent work has highlighted a striking degree of molecular and functional specialization across the cerebrovascular arterio-venous axis (Vanlandewijck, M. et al. (2018), Nature 554, 475-480). For example, arterial endothelial cells play a critical role in dynamically coupling blood flow with neural activity to meet local energetic demand (Chen, B. R., et al. (2014), J Am Hear. Assoc 3, e000787; Longden, T. A. et al. (2017), Nat Neurosci 20, 717-726. Chow, B. W. et al. (2020), Nature 579, 106-110), capillary endothelial cells actively suppress transcytotic trafficking to maintain blood-brain barrier integrity (Ben-Zvi, A. et al. (2014), Nature 509, 507-511; Andreone, B. et al. (2017), Neuron 94, 581-594; Chow, B. et al. (2017), Neuron 93, 1325-1333.e3), and venous endothelial cells appear to act as essential intermediaries in neuroimmune crosstalk (Mastorakos, P. et al. (2019), Sci Immunol 4, eaav0492; Kerfoot, S. M. et al. (2002), J Immunol 169, 1000-1006; Piccio, L. et al. (2002), J Immunol 168, 1940-1949). However, a mismatch between the expanding functions ascribed to endothelial cells and the relatively limited tools available to study them in vivo is a major obstacle to research progress. A highly efficient, endothelial-specific vector with broad tropism encompassing arterial, capillary, and venous endothelial cells would be ideally suited to accelerate neurovascular research. Similarly, the ability to effectively transduce spinal cord (Bartanusz, V., et al. (2011), Ann Neurol 70, 194-206) and retinal vasculature (Stahl, A. et al. (2010), Invest Ophthalmol Vis Sci 51, 2813-2826; Newman, E. A. (2013), J Cereb Blood Flow Metab 33, 1685-1695; Chow, B. W. et al. (2017), Neuron 93, 1325-1333.e3), widely-used systems in the field of neurovascular biology, in addition to brain vasculature would dramatically expand the potential applications of a CNS-directed, endothelial-specific vector.

Citation or identification of any document in this application is not an admission that such a document is available as prior art to the present invention.

### SUMMARY

In certain example embodiments, provided herein is a composition comprising a targeting moiety effective to increase transduction of vascular endothelial cells of the CNS vasculature, the targeting moiety comprising an n-mer motif, the n-mer motif comprising or consisting of X1-N-X3-X4-X5-X6-X7, wherein X5 is independently selected from K or R, and X1, X3, X4, X6 and X7 are independently selected from any amino acid, optionally wherein the overall charge of the n-mer motif at neutral pH is between 0 and +2; and optionally further comprising a cargo coupled to or otherwise associated with the targeting moiety.

In certain example embodiments, provided herein are compositions wherein X1, X3, X4, X6, and X7 are independently selected from the following groups wherein X1 is selected from the group consisting of G, M, T, S, N, D, L, H, P, I, V, Q, Y, W, F, A, E; X3 is selected from N, S, T, H, D, A, Y, M, Q, E, R, G, V; X4 is selected from T, V, I, A, M, S, H, W, N; X6 is selected from N, S, G, D, P, T, H, Q, A, Y; and X7 is selected from T, Y, W, N, V, I, H, M, S, G, A, Q, F, D, P, R, L.

In certain example embodiments, provided herein are compositions wherein X1, X3, X4, X6, and X7 are independently selected from the following groups wherein X1 is selected from the group consisting of G, M, T, S, N, D; X3 is selected from the group consisting of N, S, T, H, D; X4 is selected from the group consisting of T, V, I, A; X6 is selected from the group consisting of N, S, G, D, P; and X7 is selected from T, Y, W, N, V, I, H, M, S, G, A, Q, F, D, P, R, L.

In an example embodiment, provided herein are compositions wherein X1 is R or K and X3, X4, X6 and X7 are D or E.

In embodiments, provided herein are compositions wherein X1 is not R, K, or C; X3 is not W, F, K, C, I, P or, L; X4 is not Y, G, P, D, C, Q, R, K, E, F, L, or R; X6 is not R, I, W, V, F, C, L, E, or K; or X7 is not C, K, E.

In an embodiment, provided herein are compositions wherein the n-mer motif is selected from one of Table 1 to Table 6.

In embodiments, the n-mer motif is NNSTRGG (SEQ ID NO: 1), GNSARNI (SEQ ID NO: 2), GNSVRDF (SEQ ID NO: 3), or a combination thereof.

In an embodiment, provided herein are compositions wherein the targeting moiety is part of a viral capsid protein. In an embodiment, the n-mer motif is located between two amino acids of the viral capsid protein such that the n-mer is external to a viral capsid. In an embodiment, the n-mer motif is located between two amino acids of the AAV capsid protein such that the n-mer is external to a viral capsid. In an embodiment, the n-mer is a 7-mer and is inserted between amino acids 588 and 589 in an AAV9 capsid polypeptide, or in an analogous position in an AAV1, AAV2, AAV3, AAV4, AAV5, AAV6, AAV7, AAV8, AAV rh.74, AAV rh.10 capsid polypeptide.

In an embodiment, provided herein are compositions wherein the engineered AAV capsid protein comprises one or more mutations. In an example embodiment, the one or more mutations comprise K449R of AAV9, or in an analogous position in an AAV1, AAV2, AAV3, AAV4, AAV5, AAV6, AAV7, AAV8, AAV rh.74, AAV rh.10 capsid polypeptide.

In an embodiment, provided herein are compositions wherein the cargo is a polynucleotide, a morpholino, a peptide-linked morpholino, a PMO, one or more polypeptides, or ribonucleoprotein complex. In an embodiment, the polynucleotide encodes one or more polypeptides and/or a short or small hairpin RNA (shRNA) or a microRNA (miRNA). In an embodiment, the polynucleotide encodes one or more polypeptides. In an embodiment, the one or more polypeptides comprise enzymes, transport proteins or antibodies. In an embodiment, the polynucleotide encodes a CRISPR-Cas.

In a certain example embodiment, the cargo is a polynucleotide, the polynucleotide comprising one or more repeat elements that reduce or eliminate expression of the polynucleotide in a non-vascular endothelial cell of the CNS. In an embodiment, the one or more repeat elements are the hepatocyte-selective miR-122 repeat element.

In an example embodiment, provided herein is a vector system comprising one or more vectors encoding a targeting moiety comprising an n-mer, the n-mer comprising or consisting of the targeting moiety comprising an n-mer motif, the n-mer motif comprising or consisting of X1-N-X3-X4-X5-X6-X7, wherein X5 is independently selected from K or R, and X1, X3, X4, X6 and X7 are independently selected from any amino acid, optionally wherein the overall charge of the n-mer motif at neutral pH is between 0 and +2; and a cargo polynucleotide.

In an example embodiment, provided herein are vectors wherein X1, X3, X4, X6, and X7 are independently selected from the following groups: X1 is selected from the group consisting of G, M, T, S, N, D, L, H, P, I, V, Q, Y, W, F, A, E; X3 is selected from N, S, T, H, D, A, Y, M, Q, E, R, G, V; X4 is selected from T, V, I, A, M, S, H, W, N; X6 is selected from N, S, G, D, P, T, H, Q, A, Y; and X7 is selected from T, Y, W, N, V, I, H, M, S, G, A, Q, F, D, P, R, L.

In an example embodiment, provided herein are vectors wherein X1, X3, X4, X6, and X7 are independently selected from the following groups: X1 is selected from the group consisting of G, M, T, S, N, D; X3 is selected from the group consisting of N, S, T, H, D; X4 is selected from the group consisting of T, V, I, A; X6 is selected from the group consisting of N, S, G, D, P; and X7 is selected from T, Y, W, N, V, I, H, M, S, G, A, Q, F, D, P, R, L.

In an embodiment, provided herein are vectors wherein X1 is R or K and X3, X4, X6 and X7 are D or E.

In an embodiment, provided herein are vectors wherein X1 is not R, K, or C; X3 is not W, F, K, C, I, P or L; X4 is not Y, G, P, D, C, Q, R, K, E, F, L or R; X6 is not R, I, W, V, F, C, L, E, or K; X7 is not C, K, or E.

In an embodiment, provided herein are vectors wherein the n-mer is selected from any one as listed in Tables 1-6, or any combination thereof.

In an embodiment, provided herein are vectors wherein the vector encodes the targeting moiety within a viral capsid protein. In an embodiment, the n-mer motif is located between two amino acids of the viral capsid protein such that the n-mer is external to the viral capsid. In an embodiment, the n-mer motif is inserted between amino acids 588 and 589 in an AAV9 capsid polypeptide, or in an analogous position in an AAV1, AAV2, AAV3, AAV4, AAV5, AAV6, AAV7, AAV8, AAV9, AAV rh.74, AAV rh. 10 capsid polypeptide.

In an embodiment, provided herein are vectors wherein the AAV capsid protein comprises one or more mutations. In an embodiment, the one or more mutations comprise K449R of AAV9, or in an analogous position in AAV1, AAV2, AAV3, AAV4, AAV5, AAV6, AAV7, AAV8, AAV rh.74, AAV rh.10 capsid polypeptide.

In an embodiment, provided herein are vectors wherein the polynucleotide encodes an RNAi oligonucleotide. In an embodiment, the polynucleotide encodes one or more polypeptides. In an embodiment, the polypeptides include enzymes, transport proteins and antibodies.

In an embodiment, the polynucleotide encodes a CRISPR-Cas system.

In an embodiment, provided herein are vectors wherein the cargo polynucleotide further comprises one or more repeat elements that reduce or eliminate expression of the polynucleotide in a non-endothelial cell of the CNS vasculature. In an embodiment, the one or more repeat elements are the hepatocyte-selective miR-122 repeat element inserted into the 3′ UTR of the polynucleotide.

In an example embodiment, provided herein is a polypeptide encoded or produced by the vector system.

In an example embodiment, provided herein is a particle produced by the vector system.

In an example embodiment, provided herein is a cell comprising the composition, vector, polypeptide, or particle of any of the above.

In an example embodiment, is disclosed a method of delivering a cargo to endothelial cells of the CNS, lung, or kidney vasculature and/or hepatocytes comprising administering, in vivo or in vitro, any of the compositions as disclosed above, or any of the vectors disclosed above. In an embodiment, is disclosed a method of delivery wherein the cargo is a RNAi oligonucleotide, a polynucleotide encoding a polypeptide, or a polypeptide.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1B: Amino acid count matrices for the N2KR5 motif. (A) Average enrichment of AAs at certain positions, for variants conforming to the ‘*N**[K/R]**’ (N2KR5) motif i.e., the “G” cell in position 1 represents all variants conforming to the more specific motif ‘GN**[K/R]**’, and the cell is shaded/colored by the average enrichment of those matching variants in the respective screening assay. The number within each cell represents the number of variants that match this more specific motif Each shading/color mapping is constrained to remove the influence of outliers and better show the dynamic range. (B) Same as A, except for each cell (which represents a more specific motif), only variants which exceed the median assay score of all variants are counted. Cells are shaded/colored by the number of variants which pass this filter, and this number is also written as text inside each cell.

FIG. 2: Overall charge distribution of XNXX[K/R]XX variants at neutral pH. The overall 7-mer charge distribution of example XNXX[K/R]XX variants listed in Table 1-6 is plotted in the histogram.

FIG. 3: Enrichment of AAV-B130 by in vivo and in vitro selection. An AAV9 7-mer library was intravenously administered to (i) adult C57BL/6J and BALB/cJ mice at 1×10¹¹ vg/animal and (ii) human & mouse primary BMVECs and hCMEC/D3 human endothelial cells in vitro at 1×10⁴ vg/cell. Capsid mRNA was recovered from mouse brain or from cells in vitro after 21 or 3 days, respectively. The enrichment of AAV-B130 as well as AAV9 and AAV-PHP.eB controls was calculated as the log 2 of the variant reads per million (RPM) in the indicated assay divided by the variant RPM in the virus library. Each of the three variants was represented by two distinct nucleotide sequences: replicate sequences (circles) are shown along with the mean. N.D. indicates sequences not detected in the assay.

FIG. 4A-4G: AAV-B130 enables efficient transduction of brain endothelial cells across species. (A) Quantification of transduction by AAV-B130 and AAV-PHP.eB relative to AAV9 in several independent batches of mouse & human BMVECs and human CMEC/Ds assessed by luciferase activity (relative light units). (B) Representative image of AAV-B130 transduction in a sagittal section of adult C57BL/6 brain cropped to show cortex and hippocampus. The samples were collected 10 days after administration of 1×10¹¹ vg of AAV-B130 carrying a CAG-NLS-GFP-WPRE genome (C) High-magnification image of mouse liver harvested 10 days following intravenous injection of 3×10¹¹ vg of AAV-B130. Note abnormal nuclear morphology in hepatocytes expressing the highest levels of GFP. (D) AAV-B130 carrying a CAG-NLS-GFP-WPRE or CAG-NLS-GFP-miR122-3x-WPRE genome were intravenously injected into adult C57BL/6 mice at 1×10¹¹ vg/animal. Left: low-magnification images of liver collected 10 days after injection, demonstrating effective miR122-mediated suppression of transgene expression in hepatocytes. Right: Starting immediately after viral injection, mice were weighed every 24 hours for 20 consecutive days (mean±s.e.m.; n=3 animals per group). (E) AAV-BI30:CAG-NLS-GFP-miR122-3x-WPRE was intravenously administered at 1×10¹¹ vg/animal (BALB/cJ) or 1.42×10¹³ vg/kg (rat). Transduction was assessed after three (BALB/cJ) or four (rat) weeks. Representative images show AAV-B130 transduction in the cortical microvasculature of each animal. (F) AAV-BI30 or AAV-BR1 carrying a CAG-NLS-GFP-miR122-3x-WPRE construct were intravenously administered to adult C57BL/6 mice at 1×10¹¹ vg/animal. Transduction was assessed after three weeks. Representative confocal images of viral transduction in cerebral cortex; note high cell-type specificity of ERG immunostaining. (G) Quantification of endothelial transduction measured as the fraction of ERG⁺ cells expressing GFP in whole sagittal sections of brain (t₄=3.631; P=0.0221). For quantification: n=3 animals per group, mean±s.e.m.; unpaired, two-tailed t-test (* P<0.05). Scale bars are as follows: 500 μm in (D); 250 μm in (B); 100 μm in (E) and (F); 25 μm in (C).

FIG. 5: AAV-B130 is more efficient at transducing hCMEC/D3 cells than AAV9 across a wide range of doses. hCMEC/D3 cells were grown to confluence in a 24-well plate format. AAV9 or AAV-B130 carrying a CAG-NLS-GFP-miR122-WPRE genome was applied to cells at 0, 500, 1,000, 5,000, 10,000, or 50,000 vg/cell. 4 days post-treatment the cells were analyzed for GFP expression via flow cytometry. The profiles show data from a single sample from each condition but are representative of n=2 replicates.

FIG. 6: AAV-B130 transduces mouse brain endothelial cells in vivo. AAV-BI30:CAG-NLS-GFP-WPRE was intravenously administered to adult C57BL/6 mice at 1×10¹¹ vg/animal and transduction was assessed after 10 days. Representative sagittal section shown demonstrates highly efficient, endothelial-specific transduction across the brain. Scale bar shown is 1 mm.

FIG. 7: Comparison of AAV-B1I30's tropism to that of its parental vector, AAV9. AAV-B130 or AAV9 vectors carrying a CAG-NLS-GFP-WPRE genome were intravenously administered to adult C57BL/6 mice at 1×10¹¹ vg/animal and transduction was assessed after 7 days. Panels show NLS-GFP transgene expression in organs throughout the body. Scale bar shown is 200 m. Images are representative of n=3 animals per group.

FIG. 8: Following incorporation of miR122 element into viral genome, AAV-B130 can be administered at high doses without systemic toxicity. A 5×10¹¹ vg/animal dose of AAV-BI30:CAG-NLS-GFP-miR122-WPRE or saline was intravenously injected into adult C57BL/6 mice. Starting immediately after injection, mice were weighed every 24 hours for 20 consecutive days (mean±s.e.m.; n=5 animals per group). Notably, mice used for this experiment weighed an average of 21.0±1.1 grams (mean±s.d.) upon AAV-B130 administration compared to 24.0±1.4 grams in the cohort shown in FIG. 1D. This discrepancy likely explains differences in the weight-gain trajectories of mice injected with 1×10¹¹ vg and 5×10¹¹ vg doses of AAV-BI30:CAG-NLS-GFP-miR122-WPRE.

FIG. 9: Characterization of AAV-B1I30's peripheral tropism reveals preferential transduction of CNS endothelium. AAV-BI30:CAG-NLS-GFP-miR122-WPRE was intravenously administered to adult C57BL/6 mice at 1×10¹¹ vg/animal and transduction was assessed after three weeks. Representative images of AAV-B130 transduction throughout the periphery; high-zoom co-localization of GFP with endothelial markers is shown in the rightmost column. With the notable exception of lung, AAV-B130 rarely transduced endothelial cells in the microvasculature of peripheral organs—a striking contrast to efficient transduction seen throughout the CNS vasculature. Transduction observed in kidney glomeruli was non-endothelial; GFP⁺ cells are most likely mesangial cells. Relatively strong endothelial transduction in the interlobular vessels of the renal medulla and the aorta suggest that AAV-B130 may achieve widespread transduction of large-diameter arteries and veins throughout the systemic circulation. Note residual NLS-GFP expression in hepatocytes of the liver persisting in the presence of miR122 repeats in the viral genome, illustrating AAV-B1I30's potent transduction of this cell type. Scale bars are as follows: 100 μm in fourth column from left, 15 μm in fifth column, and 25 μm in aorta panel. Images are representative of n=3 animals.

FIG. 10: AAV-B130 transduces endothelial cells throughout the BALB/cJ and rat brain. AAV-BI30:CAG-NLS-GFP-miR122-WPRE was intravenously administered at 1×10¹¹ vg/animal (BALB/cJ) or 1.42×10¹³ vg/kg (Rat). Transduction was assessed after three (BALB/cJ) or four (rat) weeks. Images demonstrate endothelial expression of NLS-GFP transgene throughout brains of each model organism. Scale bars shown are 200 μm (BALB/cJ third row from left) or 100 μm (BALB/cJ rightmost row & rat).

FIG. 11: AAV-B1I30's robust endothelial transduction is consistent across brain regions. AAV-BI30:CAG-NLS-GFP-miR122-WPRE was intravenously administered to adult C57BL/6 mice at 1×10¹¹ vg/animal and transduction was assessed after three weeks. Images demonstrate high endothelial expression of NLS-GFP transgene throughout the brain. Region-specific endothelial transduction efficiency was as follows: 86±6% in cortex, 81±5% in hippocampus, 85±3% in thalamus, 83±2% in cerebellum (mean s.e.m.; n=3 animals). Compare to 84±4% efficiency measured across entire brain. Scale bars are as follows: third row from left 100 m; rightmost row 50 m.

FIG. 12A-12B: AAV-B1I30's transduction profile within the brain is highly endothelial-specific. AAV-B1I30 or AAV-BR1 vectors carrying a CAG-NLS-GFP-miR122-WPRE genome were intravenously administered to adult C57BL/6 mice at 1×10¹¹ vg/animal and transduction was assessed after three weeks. (A) Representative images of rare instances of neuronal and astrocytic transduction observed following AAV-B130 administration at the 1×10¹¹ vg dose; cell types of interest are demarcated with red arrowheads. Scale bar shown is 50 m. (B) Quantification of AAV transduction in non-endothelial (GFP⁺ERG⁻) cells per mm² of cortex using 18 μm sagittal sections of brain. An average of 0.6±0.3 and 10.8±3.0 cells/mm² (mean±s.e.m.; n=3 animals per group) were identified in AAV-B130 and AAV-BR1 administered cohorts, respectively. Consistent with previous reports, neurons constituted the majority of non-endothelial cells transduced by AAV-BR1. The data presented were compared using an unpaired, two-tailed t-test (t₄=3.37; P=0.0281).

FIG. 13A-13D: AAV-B130 efficiently transduces endothelial cells across the arterio-venous axis. AAV-B130 or AAV-BR1 carrying a CAG-NLS-GFP-miR122-WPRE construct were intravenously administered to adult C57BL/6 mice at 1×10¹¹ vg/animal. Transduction was assessed after three weeks. (A) Representative images of AAV-B130 and AAV-BR1 transduction in whole-mount preparations of the pia vasculature. Note strong GFP signal present in arteries, veins, and capillaries following AAV-B130 transduction; by contrast, GFP⁺ endothelial cells transduced by AAV2-BR1 are predominantly restricted to capillary microvessels. (B) Illustration of semi-automated image-processing workflow used to calculate arterial and venous transduction efficiency. Left: input image—note that arteries, veins, and capillaries are clearly separable based on nuclear morphology of endothelial cells and SMA expression. Middle: manual annotation of arteries and veins. Artery-vein overlap regions were intentionally omitted from analysis. Right: arterial and venous EC nuclei identified by automated Cell Profiler pipeline superimposed on ERG channel of input image. (C) Quantification of endothelial transduction measured as the fraction of ERG⁺ cells expressing GFP within manually-annotated arterial (t₄=7.172; P=0.0020) and venous (t₄=9.488; P=0.0007) vessel segments. (D) Representative two-photon z-stacks of brain vasculature imaged in live, awake mice demonstrate AAV-B1I30's robust transduction of cerebrovascular arteries, veins, and capillaries. For quantification: n=3 animals per group, mean±s.e.m.; unpaired, two-tailed t-test (** P<0.01, *** P<0.001). Scale bars are as follows: 100 μm in (A) and (B); 25 μm in (D).

FIG. 14: AAV-B1I30's efficient endothelial transduction extends to the brain's largest arteries. AAV-BI30:CAG-NLS-GFP-miR122-WPRE was intravenously administered to adult C57BL/6 mice at 5×10¹¹ vg/animal and transduction was assessed after 3 weeks. Robust, endothelial-specific transduction was observed throughout the cerebral arteries, Circle of Willis, and the head of the basilar artery. Scale bar shown is 100 m.

FIG. 15A-15E: AAV-B130 targets endothelial cells throughout the retina and spinal cord vasculature. AAV-B130 or AAV-BR1 carrying a CAG-NLS-GFP-miR122-WPRE construct were intravenously administered to adult C57BL/6 mice at 1×10¹¹ vg/animal. Transduction was assessed after three weeks. Representative low (A) and high (B) magnification images of AAV-BI30 and AAV-BR1 transduction in retina. (C) Quantification of retinal endothelial transduction measured as the fraction of ERG*cells expressing GFP in superficial plexus arteries (SP aECs: t₄=13.05; P=0.0002), intermediate plexus vessels (IP ECs: t₄=11.44; P=0.0003), deep plexus vessels (DP ECs: t₄=8.107; P=0.0013), and superficial plexus veins (SP vECs: t₄=12.45; P=0.0002). (D) Representative images of AAV-B130 and AAV-BR1 transduction in spinal cord; high-zoom co-localization of GFP with endothelial markers is shown in bottom row. (E) Quantification of endothelial transduction measured as the fraction of ERG⁺ cells expressing GFP in whole transverse sections of spinal cord (t₄=4.815; P=0.0086). For quantification: n=3 animals per group, mean±s.e.m.; unpaired, two-tailed t-test (** P<0.01, *** P<0.001). Scale bars are as follows: 100 μm in (A) and middle column of (D); 50 m in (B); 25 μm in rightmost column of (D).

FIG. 16: AAV-B1I30-mediated gene transfer enables long-term transgene expression in CNS endothelial cells. AAV-BI30:CAG-NLS-GFP-miR122-WPRE was intravenously administered to adult C57BL/6 mice at 1×10¹¹ vg/animal and transduction was assessed after approximately 5 months (152 days). Vascular counterstains displayed are as follows: ICAM2 in brain parenchyma and spinal cord; isolectin in retina; and ERG in pia vasculature. High-zoom co-localization with GFP shown in rightmost column. Scale bars are as follows: middle row 200 m; rightmost row 50 m. Images are representative of n=3 mice.

FIG. 17A-17E: AAV-B130 can be leveraged to achieve efficient endothelial-specific genetic manipulation. (A) AAV-BI30:CAG-Cre-miR122-WPRE was intravenously administered to adult Ai9 Cre-dependent reporter mice at 1×10¹¹ vg/animal and recombination was assessed after 12 days. Robust tdTomato expression was observed throughout the brain microvasculature (left) as well as arteries and veins situated at the pia surface (right). Representative low (B) and high (C) magnification images of B130-mediated Ai9 recombination in the cortical microvasculature; co-localization with endothelial-specific markers demonstrates cell-type-specificity. Recombination efficiency—measured as the fraction of ERG⁺ cells expressing tdTomato—was 94±1% (mean±s.e.m.; n=3 animals) in this brain region. (D) A 1×10¹¹ vg/animal dose of AAV-BI30:CAG-Cre-miR122-WPRE or saline was intravenously administered to adult Cav1^{fl/fl} mice and Caveolin-1 protein levels were assessed after four weeks. Representative images of brain microvasculature demonstrate strong reduction of endothelial Caveolin-1 in AAV-B130-injected animals. The majority of endothelial cells in these animals exhibited a spectrum of moderate to near-complete protein loss (red arrow), likely reflecting variable viral genome count and consequent stage of protein turnover. A small fraction of endothelial cells showed no evidence of Caveolin-1 loss (blue arrow). (E) Representative images of heart microvasculature in AAV-BI30:CAG-Cre-miR122-WPRE and saline-injected mice; unaltered endothelial Caveolin-1 expression in the hearts of AAV-B130-injected animals illustrates CNS-directed loss-of-function achieved with this experimental approach. All images are representative of n=3 animals per group. Scale bars are as follows: 250 μm in left panel of (A); 100 μm in right panel of (A); 50 μm in (B) and (E); 20 μm in (D); 5 μm in (C).

FIG. 18: AAV-B130-mediated Cre delivery drives efficient recombination throughout brain vasculature. AAV-BI30:CAG-Cre-miR122-WPRE was intravenously administered to adult Ai9 Cre-dependent reporter mice at 1×10¹¹ vg/animal and recombination was assessed after 12 days. Representative sagittal section shown demonstrates highly efficient, endothelial-specific recombination across brain regions. Scale bar shown is 1 mm.

FIG. 19: Site saturation mutagenesis at AAV-B130 597Q (AAV9 position 590Q) identifies variants that outperform AAV-B130 in their ability to transduce cells in the marmoset brain. The heat map shows the mean enrichment of 10 replicates for each AAV-B130 variant in the indicated brain region. AAV-B130 Q597 to D, E, F, G, P, S, T, or Y variants are more enriched than AAV-B130 across most brain regions.

FIG. 20: AAV-B130 production yields. An average of 6.62×10^{11±}3.16×10¹¹ DNAse-resistant viral genomes (mean s.d.) were obtained from preparations of AAV-B130 in 15 cm tissue culture plates. Each data point represents an individual transgene packaged by AAV-B1I30. Yields are shown on a log₁₀ scale.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to International Patent Publication WO 2020/160337 filed Jan. 30, 2020, the contents of which are incorporated specifically herein by reference.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide targeting moieties having an enhanced selectivity for endothelial cells of the central nervous system (CNS) vasculature, including spinal and retinal vasculature. These targeting moieties may be incorporated into particles, such as viral capsid based delivery particles, to confer a tropism on the delivery particles and enhance transduction of endothelial cells of the CNS vasculature. Accordingly, embodiments disclosed herein provide compositions capable of delivering cargos with enhanced selectivity and efficiency to the CNS vasculature. Embodiments disclosed herein also provide vector systems for the generation and loading of such delivery particles with a cargo. Likewise, embodiments disclosed herein provide methods for use of such compositions to target CNS endothelial cells, in vitro and in vivo, with implications for both therapeutic and research purposes.

CNS endothelial cells line the luminal face of blood vessels, including the blood-brain-barrier, which orchestrate key homeostatic processes. Situated at the interface of the nervous and circulatory systems, endothelial cells actively regulate the biochemical composition of the CNS microenvironment, the transmission of inflammatory and immune signals and the dynamic coupling of blood flow to meet local neuronal energetic domain. Furthermore, endothelial dysfunction is increasingly implicated in a wide range of neurological diseases. Thus, embodiments disclosed provide a selective and high-efficiency delivery system for this critical cell and tissue type.

Additional feature and advantages of the aforementioned embodiments are further described below.

### Targeting Moieties and Compositions Thereof

In example embodiments, compositions are provided herein comprising a targeting moiety with an enhanced tropism for endothelial cells of the CNS vasculature. This targeting moiety may be coupled directly to a cargo to be delivered such as an oligonucleotide or polypeptide. Alternatively, the targeting molecule may be incorporated into a delivery particle to confer tropism for endothelial cells of the CNS vasculature on the delivery particle. A non-limiting example of delivery particle is a viral capsid particle. In such embodiments, the targeting moiety may be incorporated into a viral capsid polypeptide such that the targeting moiety is incorporated into the assembled viral capsid. However, other particle delivery systems where the targeting moiety may be incorporated or attached, for example on exosomes or liposomes, are also envisioned and encompassed as alternative embodiments herein.

The targeting moiety comprise a n-mer motif. The n-mer motif may comprise or consists of X1-X2-X3-X4-X5-X6-X7, where position X2 is an N (Asn) and position X5 is either a K (Lys) or R (Arg) and positions X1, X3, X4, X6, and X7 are any amino acid. It should be understood that any reference to any amino acid is intended to encompass any natural amino acid as well as any amino acid mimetic having similar physical and chemical characteristics to naturally occurring amino acids. In one example embodiment, X5 may also be any amino acid mimetic capable of providing a positive a positive charge like that of K or R. In an example embodiment, the composition of the n-mer motif may be selected such that overall charge of the n-mer motif at neutral pH is between 0 and +2.

In one example embodiment, X1, X3, X4, X6, and X7 are independently selected from the following groups: X1 is selected from the group consisting of G, M, T, S, N, D, L, H, P, I, V, Q, Y, W, F, A, E; X3 is selected from the group consisting of amino acids N, S, T, H, D, A, Y, M, Q, E, R, G, V; X4 is selected from the group consisting of T, V, I, A, M, S, H, W, N; X6 is selected from the group consisting of N, S, G, D, P, T, H, Q, A, Y. X7 is selected from the group consisting ofT, Y, W, N, V, I, H, M, S, G, A, Q, F, D, P, R, L.

In another example embodiment, the n-mer motif of X1, X3, X4, X6, and X7 are independently selected from the following groups: X1 is selected from the group consisting of G, M, T, S, N, D; X3 is selected from the group consisting of N, S, T, H, D; X4 is selected from the group consisting of T, V, I, A; X6 is selected from the group consisting of N, S, G, D, P; and X7 is selected from the group consisting of T, Y, W, N, V, I, H, M, S, G, A, Q, F, D, P, R, L.

In one example embodiment, if X1 is R or K then at least one of X3, X4, X6 and X7 are D or E.

In an example embodiment, the composition of the n-mer at position X1 is not R, K or C; X3 is not W, F, K, C, I, P, or L; X4 is not Y, G, P, D, C, Q, R, K, E, F, L, or R; X6 is not R, I, W, V, F, C, L, E, or K; and X7 is not C, K, E.

In an example embodiment, the targeting moiety can be further defined by the formula as: X1-N-X3-(T, V, I, A, M, S, H, W, N)-(K, R)-X6-X7, where position X2 is an N, position X4 is either a T, V, I, A, M, S, H, W, or N, position X5 is K or R and positions X1, X3, X6, X7 are any amino acid.

In an embodiment, the targeting moiety is further defined by the formula as: X1-X2-N-X3-(T, V, I, A)-(K, R)-X6-X7, where position X2 is an N, position X4 is either a T, V, I, A, position X5 is either K or R and positions X1, X3, X6, X7 are any amino acid.

In an embodiment, the targeting moiety is further defined by the formula as: X1-X2-N-X3-(T, V, I, A)-(K, R)-X6-X7, where position X2 is N, position X4 is T, V, I, A, position X5 is K or R.

In an example embodiment, the targeting moiety is further defined by: X1 is selected from (G, M, T, S, N, D) or (L, H, P, I, V, Q, Y, W, F, A, E) or (R, K, E, C) or (R, K), X2 is N, wherein at position X3 is either (N, S, T, H, D) or (A, Y, M, Q, E, R, G, V) or (W, F, K, C, I, P, L); at position X4 is either (T, V, I, A) or (M, S, H, W, N) or (Y, G, P, D, C, Q, R, K, E, F, L, R); position X5 is either (K) or (R); position X6, is either (N, S, G, D, P) or (T, H, Q, A, Y) but not (R, I, W, V, F, C, L, E, K); at position X7, is either (T, Y, W, N, V, I, H, M, S, G, A, Q, F, D, P, R, L) but not (C, K, E); and wherein when X1 is R or K, X3, X4, X6 or X7 is selected from D or E.

In one example embodiment, the n-mer is selected from any one of the n-mer motifs as listed in Tables 1-6 below.

In one example embodiments, the targeting motif is. NNSTRGG (SEQ ID NO: 1) (B1I30). In another example embodiment the targeting motif is GNSARNI (SEQ ID NO: 2) (B1I33). In another example embodiment and BI55: GNSVRDF (SEQ ID NO: 3).

Example embodiments further include polynucleotides encoding any of the above-mentioned targeting moieties.

In an embodiments, the targeting moiety can be used to increase transduction in target cells. The increase in transduction efficiency of the targeting moiety to a cell may be compared to a composition that does not contain the targeting moiety, for example inclusion of one or more targeting moieties in a composition can result in an increase in transduction and or transduction efficiency by 10%, 20%, 30%, 40%, 50%, 60%, 70%, 80%, 90%, 100% or more. In an exemplary embodiment, the increase in transduction and or transduction efficiency is two-fold, three-fold, four-fold, five-fold, six-fold, seven-fold, eight-fold, nine-fold, ten-fold, 20-fold, 30-fold, 40-fold, 50-fold, 60-fold, 70-fold, 80-fold or more relative to a composition lacking the targeting moiety. In one embodiment, the transduction and/or transduction efficiency is increased or enhanced in endothelial cells. In one embodiment, there is an increase in endothelial cells of the vasculature, for example, the central nervous system vasculature. In embodiments, the transduction and/or transduction efficiency is increased or enhanced in cells of the central nervous system. In embodiments, the transduction and/or transduction efficiency is increased or enhanced in hepatocytes or in endothelial cells of the kidney or of the muscle. In an embodiment, the composition comprising a targeting moiety is selective to a target cell as compared to other cell types and/or other virus particles. As used herein, ‘selective’ and ‘cell-selective’ refers to preferential targeting for cells as compared to other cell types. Preferably, the targeting moiety is selective for a desired target (e.g., cell, organ, system e.g., large diameter arteries and veins, brain, retina and spinal cord microvasculature, species) or set of targets by at least 2:1, 3:1, 4:1, 5:1, 6:1 7:1, 8:1, 9:1. 10:1 or more; or 10%, 20%, 30%, 40%, 50%, 60%, 70%, 75%, 80%, 85%, 90% or more, relative to other targets or cells (e.g., microvasculature of peripheral organs such as the kidney). In an embodiment, the composition comprising a targeting moiety described herein can have an increased uptake, delivery rate, transduction rate, efficiency, amount, or a combination thereof in a target cell (e.g., endothelial cells across the arterio-venous axis in brain, retina, and spinal cord vasculature) as compared to other cell types (e.g., muscle cells) and/or other virus particles (e.g., AAVs not containing the targeting moiety) and other compositions that do not contain the cell-selective n-mer motif of the present invention.

### Engineered Viral Capsids

Described herein are various embodiments of engineered viral capsids, such as adeno-associated virus (AAV) capsids, that can be engineered to confer cell-selective tropism, such as CNS vascular endothelial cell tropism, to an engineered viral particle. Engineered viral capsids can be lentiviral, retroviral, adenoviral, or AAV capsids. The engineered capsids can be included in an engineered virus particle (e.g., an engineered lentiviral, retroviral, adenoviral, or AAV virus particle), and can confer cell-selective tropism to the engineered viral particle. The engineered viral capsids described herein can include one or more engineered viral capsid proteins described herein. The engineered viral capsids described herein can include one or more engineered viral capsid proteins described herein that can contain one or more targeting moieties as described above.

The engineered viral capsids can be variants of wild-type viral capsid. For example, in some embodiments, the engineered AAV capsids can be variants of wild-type AAV capsids. In some embodiments, the wild-type AAV capsids can be composed of VP1, VP2, VP3 capsid proteins or a combination thereof. In other words, the engineered AAV capsids can include one or more variants of a wild-type VP1, wild-type VP2, and/or wild-type VP3 capsid proteins. In some embodiments, the serotype of the reference wild-type AAV capsid can be AAV-1, AAV-2, AAV-3, AAV-4, AAV-5, AAV-6, AAV-8, AAV-9 or any combination thereof. In some embodiments, the serotype of the wild-type AAV capsid can be AAV-9. The engineered AAV capsids can have a different tropism than that of the reference wild-type AAV capsid.

In an example embodiment, the targeting moieties disclosed herein can be inserted between two amino acids in the wild-type viral protein (VP) (or capsid protein). In some embodiments, the n-mer motif can be inserted between two amino acids in a variable amino acid region in a viral capsid protein.

In some embodiments, the n-mer motif can be inserted between two amino acids in a variable amino acid region in an AAV capsid protein. The core of each wild-type AAV viral protein contains an eight-stranded beta-barrel motif (betaB to betaI) and an alpha-helix (alphaA) that are conserved in autonomous parovirus capsids (see e.g., DiMattia et al. 2012. J. Virol. 86(12):6947-6958). Structural variable regions (VRs) occur in the surface loops that connect the beta-strands, which cluster to produce local variations in the capsid surface. AAVs have 12 variable regions (also referred to as hypervariable regions) (see e.g., Weitzman and Linden. 2011. “Adeno-Associated Virus Biology.” In Snyder, R. O., Moullier, P. (eds.) Totowa, NJ: Humana Press). In one example embodiment, one or more targeting moieties can be inserted between two amino acids in one or more of the 12 variable regions in the wild-type AVV capsid proteins. In one example embodiment, the one or more targeting motifs can be each be inserted between two amino acids in VR-I, VR-II, VR-III, VR-IV, VR-V, VR-VI, VR-VII, VR-III, VR-IX, VR-X, VR-XI, VR-XII, or a combination thereof. In one example embodiment, the engineered capsid can have a 7-mer motif inserted between amino acids 588 and 589 of an AAV9 viral protein. SEQ ID NO: 1 is a reference AAV9 capsid sequence for at least referencing the insertion sites discussed above. It will be appreciated that targeting moieties can be inserted in analogous positions in AAV viral proteins of other serotypes, such as but not limited to, AAV1, AAV2, AAV3, AAV4, AAV5, AAV6, AAV7, AAV8, AAV9, AAV rh.74, AAV rh.10 capsid polypeptide. In some embodiments as previously discussed, the targeting moieties can be inserted between any two contiguous amino acids within the AAV viral protein and in some embodiments the insertion is made in a variable region.

In one example embodiment, the first 1, 2, 3, or 4 amino acids of an n-mer motif can replace 1, 2, 3, or 4 amino acids of a polypeptide into which it is inserted and preceding the insertion site. Using an AAV as another non-limiting example, one or more of the n-mer motifs can be inserted into e.g., an AAV9 capsid polypeptide between amino acids 588 and 589 and the insert can replace amino acids 586, 587, and 588 such that the amino acid immediately preceding the n-mer motif after insertion is residue 585. It will be appreciated that this principle can apply in any other insertion context and is not necessarily limited to insertion between residues 588 and 589 of an AAV9 capsid or equivalent position in another AAV capsid. It will further be appreciated that in some embodiments, no amino acids in the polypeptide into which the targeting moiety is inserted are replaced by the targeting moiety.

The engineered viral capsid and/or capsid proteins can be encoded by one or more engineered viral capsid polynucleotides. In some embodiments, the engineered viral capsid polynucleotide is an engineered AAV capsid polynucleotide, engineered lentiviral capsid polynucleotide, engineered retroviral capsid polynucleotide, or engineered adenovirus capsid polynucleotide. In some embodiments, an engineered viral capsid polynucleotide (e.g., an engineered AAV capsid polynucleotide, engineered lentiviral capsid polynucleotide, engineered retroviral capsid polynucleotide, or engineered adenovirus capsid polynucleotide) can include a 3′ polyadenylation signal. The polyadenylation signal can be an SV40 polyadenylation signal.

### Further Capsid Modifications

In one example embodiment, the viral capsid protein may comprise one or more mutations relative to wild type. In one example embodiment, the one or more mutations comprise K449R in AAV9, or in an analogous position in an AAV1, AAV2, AAV3, AAV4, AAV5, AAV6, AAV7, AAV8, AAV rh.74, AAV rh.10 capsid polypeptide.

### Engineered Vectors and Vector Systems

Also provided herein are vectors and vector systems that can contain one or more of the engineered polynucleotides described herein that can encode one or more of the n-mer motifs of the present invention, including but not limited to, engineered viral polynucleotides (e.g., engineered AAV polynucleotides). As used in this context, engineered viral capsid polynucleotides refers to any one or more of the polynucleotides described herein capable of encoding an engineered viral capsid as described elsewhere herein and/or polynucleotide(s) capable of encoding one or more engineered viral capsid proteins described elsewhere herein. Further, where the vector includes an engineered viral capsid polynucleotide described herein, the vector can also be referred to and considered an engineered vector or system thereof although not specifically noted as such. In embodiments, the vector can contain one or more polynucleotides encoding one or more elements of an engineered viral capsid described herein. The vectors and systems thereof can be useful in producing bacterial, fungal, yeast, plant cells, animal cells, and transgenic animals that can express one or more components of the engineered viral capsid, particle, or other compositions described herein. Within the scope of this disclosure are vectors containing one or more of the polynucleotide sequences described herein. One or more of the polynucleotides that are part of the engineered viral capsid and system thereof described herein can be included in a vector or vector system.

In some embodiments, the vector can include an engineered viral (e.g., AAV) capsid polynucleotide having a 3′ polyadenylation signal. In some embodiments, the 3′ polyadenylation is an SV40 polyadenylation signal. In some embodiments, the vector does not have splice regulatory elements. In some embodiments, the vector includes one or more minimal splice regulatory elements. In some embodiments, the vector can further include a modified splice regulatory element, wherein the modification inactivates the splice regulatory element. In some embodiments, the modified splice regulatory element is a polynucleotide sequence sufficient to induce splicing, between a rep protein polynucleotide and the engineered viral (e.g., AAV) capsid protein variant polynucleotide. In some embodiments, the polynucleotide sequence can be sufficient to induce splicing is a splice acceptor or a splice donor. In some embodiments, the viral (e.g., AAV) capsid polynucleotide is an engineered viral (e.g., AAV) capsid polynucleotide as described elsewhere herein. In some embodiments, the vector does not include one or more minimal splice regulatory elements, modified splice regulatory agent, splice acceptor, and/or splice donor.

The vectors and/or vector systems can be used, for example, to express one or more of the engineered viral (e.g., AAV) capsid and/or other polynucleotides in a cell, such as a producer cell, to produce engineered viral (e.g., AAV) particles and/or other compositions (e.g. polypeptides, particles, etc.) containing an engineered viral (e.g., AAV) capsid or other composition containing an n-mer motif of the present invention described elsewhere herein. Other uses for the vectors and vector systems described herein are also within the scope of this disclosure. In general, and throughout this specification, the term is a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include adeno-associated viruses, and types of such vectors can also be selected for targeting particular types of cells, such as those engineered viral (e.g., AAV) vectors containing an engineered viral (e.g., AAV) capsid polynucleotide with a desired cell-selective tropism. These and other embodiments of the vectors and vector systems are described elsewhere herein.

In some embodiments, the vector can be a bicistronic vector. In some embodiments, a bicistronic vector can be used for one or more elements of the engineered viral (e.g., AAV) capsid system described herein. In some embodiments, expression of elements of the engineered viral (e.g., AAV) capsid system described herein can be driven by a suitable constitutive or tissue specific promoter. Where the element of the engineered viral (e.g., AAV)capsid system is an RNA, its expression can be driven by a Pol III promoter, such as a U6 promoter. In some embodiments, the two are combined.

### Cell-Based Vector Amplification and Expression

Vectors can be designed for expression of one or more elements of the engineered viral (e.g., AAV) capsid system or other compositions containing a target motif of the present invention described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. The vectors can be viral-based or non-viral based. In some embodiments, the suitable host cell is a eukaryotic cell. In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stb12, Stb13, Stb14, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2 plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more elements of an engineered AAV capsid system so as to drive expression of the one or more elements of the engineered AAV capsid system described herein.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more elements of an engineered viral (e.g., AAV) capsid system or other composition containing an n-mer motif described herein are introduced into a host cell such that expression of the elements of the engineered delivery system described herein direct formation of an engineered viral (e.g., AAV) capsid system or other composition containing an n-mer motif described herein (including but not limited to an engineered gene transfer agent particle, which is described in greater detail elsewhere herein). For example, different elements of the engineered viral (e.g., AAV) capsid system or other composition containing an n-mer motif described herein can each be operably linked to separate regulatory elements on separate vectors. RNA(s) of different elements of the engineered delivery system described herein can be delivered to an animal or mammal or cell thereof to produce an animal or mammal or cell thereof that constitutively or inducibly or conditionally expresses different elements of the engineered viral (e.g., AAV) capsid system or other composition containing an n-mer motif described herein that incorporates one or more elements of the engineered viral (e.g., AAV) capsid system or other composition containing an n-mer motif described herein or contains one or more cells that incorporates and/or expresses one or more elements of the engineered viral (e.g., AAV) capsid system or other composition containing an n-mer motif described herein.

In some embodiments, two or more of the elements expressed from the same or different regulatory element(s), can be combined in a single vector, with one or more additional vectors providing any components of the system not included in the first vector. Engineered polynucleotides of the present invention that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more engineered viral (e.g., AAV) capsid proteins or other composition containing an n-mer motif described herein, embedded within one or more intron sequences (e.g., each in a different intron, two or more in at least one intron, or all in a single intron). In some embodiments, the engineered polynucleotides of the present invention (including but not limited to engineered viral polynucleotides) can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In embodiments, the polynucleotides and/or vectors thereof described herein (including, but not limited to, the engineered AAV capsid polynucleotides of the present invention) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, brain), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. In some embodiments, the regulated promoter is a tissue specific promoter as previously discussed elsewhere herein. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g., APOA2, SERPIN A1 (hAAT), CYP3A4, and MIR122), pancreatic cell promoters (e.g., INS, IRS2, Pdx1, Alx3, Ppy), cardiac specific promoters (e.g. Myh6 (alpha MHC), MYL2 (MLC-2v), TNI3 (cTnl), NPPA (ANF), Slc8al (Ncx1)), central nervous system cell promoters (SYN1, GFAP, INA, NES, MOBP, MBP, TH, FOXA2 (HNF3 beta)), skin cell specific promoters (e.g., FLG, K14, TGM3), immune cell specific promoters, (e.g. ITGAM, CD43 promoter, CD14 promoter, CD45 promoter, CD68 promoter), urogenital cell specific promoters (e.g., Pbsn, Upk2, Sbp, Fer114), endothelial cell specific promoters (e.g., ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g., Desmin). Other tissue and/or cell specific promoters are discussed elsewhere herein and can be generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

In some embodiments, the vector or system thereof can include one or more elements capable of translocating and/or expressing an engineered polynucleotide of the present invention (e.g., an engineered viral (e.g. AAV) capsid polynucleotide) to/in a specific cell component or organelle. Such organelles can include, but are not limited to, nucleus, ribosome, endoplasmic reticulum, golgi apparatus, chloroplast, mitochondria, vacuole, lysosome, cytoskeleton, plasma membrane, cell wall, peroxisome, centrioles, etc.

### Selectable Markers and Tags

One or more of the engineered polynucleotides of the present invention (e.g., an engineered viral (e.g., AAV) capsid polynucleotide) can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the engineered polynucleotide of the present invention (e.g., an engineered viral (e.g. AAV) capsid polynucleotide) such that the selectable marker polypeptide, when translated, is inserted between two amino acids between the N- and C-terminus of an engineered polypeptide (e.g., the engineered AAV capsid polypeptide) or at the N- and/or C-terminus of the engineered polypeptide (e.g., an engineered AAV capsid polypeptide). In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the engineered AAV capsid system described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the engineered AAV capsid system or other compositions and/or systems described herein via suitable linker, such as a glycine or glycine serine linkers as short as GS or GG up to (GGGGG)₃ (SEQ ID NO: 4) or (GGGGS)₃ (SEQ ID NO: 5). Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to selective cells, tissues, organs, etc. In some embodiments, the targeting moiety encoding polynucleotides can be included in the vector or vector system such that the engineered polynucleotide(s) of the present invention (e.g., an engineered viral (e.g., AAV) capsid polynucleotide(s)) and/or products expressed therefrom include the targeting moiety and can be targeted to selective cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated engineered polynucleotide(s) of the present invention, the engineered polypeptides, or other compositions of the present invention described herein, to select cells, tissues, organs, etc. In some embodiments, the select cells are muscle cells.

### Cell-Free Vector and Polynucleotide Expression

In some embodiments, the polynucleotide(s) encoding a targeting motif of the present invention can be expressed from a vector or suitable polynucleotide in a cell-free in vitro system. In some embodiments, the polynucleotide encoding one or more features of the engineered AAV capsid system can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenolpyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems, transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the polynucleotide encoding a targeting motif of the present invention and/or other polynucleotides described herein can be codon optimized. In some embodiments, polynucleotides of the engineered AAV capsid system described herein can be codon optimized. In some embodiments, one or more polynucleotides contained in a vector (“vector polynucleotides”) described herein that are in addition to an optionally codon optimized polynucleotide encoding an n-mer motif, including, but not limited to, embodiments of the engineered AAV capsid system described herein, can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton BR, J Mol Evol. 1998 April; 46(4):449-59.

The vector polynucleotide can be codon optimized for expression in a select cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. In some embodiments, the polynucleotide is codon optimized for a specific cell type or types. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Non-Viral Vectors and Carriers

In some embodiments, the vector is a non-viral vector or carrier. In some embodiments, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with an engineered capsid polynucleotide (e.g., an engineered AAV capsid polynucleotide) or other composition of the present invention described herein and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid or polynucleotide molecule or composition that be attached to or otherwise interact with a polynucleotide to be delivered, such as an engineered AAV capsid polynucleotide of the present invention.

### Naked Polynucleotides

In some embodiments, one or more engineered AAV capsid polynucleotides or other polynucleotides of the present invention described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered AAV capsid polynucleotides or other polynucleotides of the present invention described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the engineered AAV capsid polynucleotide(s) or other polynucleotides of the present invention. In some embodiments, the naked polynucleotide can contain other nucleic acids and/or polynucleotides in addition to the engineered AAV capsid polynucleotide(s) or other polynucleotides of the present invention described elsewhere herein. The naked polynucleotides can include one or more elements of a transposon system. Transposons and system thereof are described in greater detail elsewhere herein.

### Non-Viral Polynucleotide Vectors

In some embodiments, one or more of the engineered AAV capsid polynucleotides or other polynucleotides of the present invention can be included in a non-viral polynucleotide vector. Suitable non-viral polynucleotide vectors include, but are not limited to, transposon vectors and vector systems, plasmids, bacterial artificial chromosomes, yeast artificial chromosomes, AR(antibiotic resistance)-free plasmids and miniplasmids, circular covalently closed vectors (e.g., minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In embodiments, the S/MAR sequence is located downstream of an actively transcribed polynucleotide (e.g., one or more engineered AAV capsid polynucleotides or other polynucleotides or molecules of the present invention) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the engineered AAV capsid polynucleotide(s) or other polynucleotides, or molecules of the present invention described herein flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell, the transposase can be expressed from the second vector and can transpose the material between the TIRs on the first vector (e.g., the engineered AAV capsid polynucleotide(s) or other polynucleotides or molecules of the present invention) and integrate it into one or more positions in the host cell's genome. In some embodiments, the transposon vector or system thereof can be configured as a gene trap. In some embodiments, the TIRs can be configured to flank a strong splice acceptor site followed by a reporter and/or other gene (e.g., one or more of the engineered AAV capsid polynucleotide(s) or other polynucleotides or molecules of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include Sleeping Beauty transposon system (Tcl/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tcl/mariner superfamily) (see e.g., Miskey et al. 2003 Nucleic Acid Res. 31(23):6873-6881) and variants thereof.

### Chemical Carriers

In some embodiments the engineered AAV capsid polynucleotide(s) or other polynucleotides or other molecules of the present invention described herein can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the engineered AAV capsid polynucleotide(s) of the present invention), (2) those capable of targeting specific or select cells, (3) those capable of increasing delivery of the polynucleotide or other molecules (such as the engineered AAV capsid polynucleotide(s)) of the present invention to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the compositions (including particles, polypeptides, polynucleotides, and other compositions described herein) present invention described herein. Suitable sizes include macro-, micro-, and nano-sized particles.

In some embodiments, the non-viral carrier can be an inorganic particle. In some embodiments, the inorganic particle, can be a nanoparticle. The inorganic particles can be configured and optimized by varying size, shape, and/or porosity. In some embodiments, the inorganic particles are optimized to escape from the reticulo endothelial system. In some embodiments, the inorganic particles can be optimized to protect an entrapped molecule from degradation. The suitable inorganic particles that can be used as non-viral carriers in this context can include, but are not limited to, calcium phosphate, silica, metals (e.g., gold, platinum, silver, palladium, rhodium, osmium, iridium, ruthenium, mercury, copper, rhenium, titanium, niobium, tantalum, and combinations thereof), magnetic compounds, particles, and materials, (e.g., supermagnetic iron oxide and magnetite), quantum dots, fullerenes (e.g., carbon nanoparticles, nanotubes, nanostrings, and the like), and combinations thereof. Other suitable inorganic non-viral carriers are discussed elsewhere herein.

In some embodiments, the non-viral carrier can be lipid-based. Suitable lipid-based carriers are also described in greater detail herein. In some embodiments, the lipid-based carrier includes a cationic lipid or an amphiphilic lipid that is capable of binding or otherwise interacting with a negative charge on the polynucleotide to be delivered (e.g., such as an engineered AAV capsid polynucleotide of the present invention). In some embodiments, chemical non-viral carrier systems can include a polynucleotide (such as the engineered AAV capsid polynucleotide(s)) or other composition or molecule of the present invention) and a lipid (such as a cationic lipid). These are also referred to in the art as lipoplexes. Other embodiments of lipoplexes are described elsewhere herein. In some embodiments, the non-viral lipid-based carrier can be a lipid nano emulsion. Lipid nano emulsions can be formed by the dispersion of an immiscible liquid in another stabilized emulsifying agent and can have particles of about 200 nm that are composed of the lipid, water, and surfactant that can contain the polynucleotide to be delivered (e.g., the engineered AAV capsid polynucleotide(s) of the present invention). In some embodiments, the lipid-based non-viral carrier can be a solid lipid particle or nanoparticle.

In some embodiments, the non-viral carrier can be peptide-based. In some embodiments, the peptide-based non-viral carrier can include one or more cationic amino acids. In some embodiments, 35 to 40, 45, 50, 55, 60, 65, 70, 75, 80, 85, 90, 95, 99 or 100% of the amino acids are cationic. In some embodiments, peptide carriers can be used in conjunction with other types of carriers (e.g., polymer-based carriers and lipid-based carriers to functionalize these carriers). In some embodiments, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g., US Pat. Pub. 2017/0079916 whose techniques and compositions can be adapted for use with the engineered AAV capsid polynucleotides of the present invention), polymethacrylate, and combinations thereof.

In some embodiments, the non-viral carrier can be configured to release an engineered delivery system polynucleotide that is associated with or attached to the non-viral carrier in response to an external stimulus, such as pH, temperature, osmolarity, concentration of a specific molecule or composition (e.g., calcium, NaCl, and the like), pressure and the like. In some embodiments, the non-viral carrier can be a particle that is configured includes one or more of the engineered AAV capsid polynucleotides or other compositions of the present invention describe herein and an environmental triggering agent response element, and optionally a triggering agent. In some embodiments, the particle can include a polymer that can be selected from the group of polymethacrylates and polyacrylates. In some embodiments, the non-viral particle can include one or more embodiments of the compositions microparticles described in US Pat. Pubs. 20150232883 and 20050123596, whose techniques and compositions can be adapted for use in the present invention.

In some embodiments, the non-viral carrier can be a polymer-based carrier. In some embodiments, the polymer is cationic or is predominantly cationic such that it can interact in a charge-dependent manner with the negatively charged polynucleotide to be delivered (such as the engineered AAV capsid polynucleotide(s) of the present invention). Polymer-based systems are described in greater detail elsewhere herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as an engineered AAV capsid polynucleotide, cargo, or other composition or molecule of the present invention, into a virus particle and producing said virus particle when used alone or with one or more other viral vectors (such as in a viral vector system). Viral vectors and systems thereof can be used for producing viral particles for delivery of and/or expression and/or generation of one or more compositions of the present invention described herein (including, but not limited to, any viral particle and associated cargo). The viral vector can be part of a viral vector system involving multiple vectors. In some embodiments, systems incorporating multiple viral vectors can increase the safety of these systems. Suitable viral vectors can include adenoviral-based vectors, adeno associated vectors, helper-dependent adenoviral (HdAd) vectors, hybrid adenoviral vectors, and the like. Other embodiments of viral vectors and viral particles produce therefrom are described elsewhere herein. In some embodiments, the viral vectors are configured to produce replication incompetent viral particles for improved safety of these systems.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be serotype 2, 5, or 9. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261. The engineered AAV capsids can be included in an adenoviral vector to produce adenoviral particles containing said engineered AAV capsids.

In some embodiments, the vector can be a helper-dependent adenoviral vector or system thereof. These are also referred to in the field as “gutless” or “gutted” vectors and are a modified generation of adenoviral vectors (see e.g., Thrasher et al. 2006. Nature. 443:E5-7). In embodiments of the helper-dependent adenoviral vector system, one vector (the helper) can contain all the viral genes required for replication but contains a conditional gene defect in the packaging domain. The second vector of the system can contain only the ends of the viral genome, one or more engineered AAV capsid polynucleotides, and the native packaging recognition signal, which can allow selective packaged release from the cells (see e.g., Cideciyan et al. 2009. N Engl J Med. 361:725-727). Helper-dependent Adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the engineered AAV capsid polynucleotides described herein. In some embodiments, the polynucleotide to be delivered via the viral particle produced from a helper-dependent adenoviral vector or system thereof can be up to about 38 kb. Thus, in some embodiments, an adenoviral vector can include a DNA polynucleotide to be delivered that can range in size from about 0.001 kb to about 37 kb (see e.g. Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use in the engineered AAV capsid system of the present invention. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the engineered AAV capsid system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the engineered AAV capsid system of the present invention.

### Adeno-Associated Vectors

In an embodiment, the engineered vector or system thereof can be an adeno-associated vector (AAV). See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments, the AAV can integrate into a specific or preferred site on chromosome 19 of a human cell with no observable side effects. In some embodiments, the capacity of the AAV vector, system thereof, and/or AAV particles can be up to about 4.7 kb. The AAV vector or system thereof can include one or more engineered capsid polynucleotides described herein.

The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof. In some embodiments, the promoter can be a tissue specific promoter as previously discussed. In some embodiments, the tissue specific promoter can drive expression of an engineered capsid AAV capsid polynucleotide described herein.

The AAV vector or system thereof can include one or more polynucleotides that can encode one or more capsid proteins, such as the engineered AAV capsid proteins described elsewhere herein. The engineered capsid proteins can be capable of assembling into a protein shell (an engineered capsid) of the AAV virus particle. The engineered capsid can have a cell-, tissue,- and/or organ-selective tropism.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E40RF6, and VA RNAs. In some embodiments, a producing host cell line expresses one or more of the adenovirus helper factors.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5, AAV-9 or any combination thereof. One can select the AAV of the AAV with regard to the cells to be targeted; e.g., one can select AAV serotypes 1, 2, 5, 9 or a hybrid capsid AAV-1, AAV-2, AAV-5, AAV-9 or any combination thereof for targeting brain and/or neuronal cells; and one can select AAV-4 for targeting cardiac tissue; and one can select AAV-8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. See also Srivastava. 2017. Curr. Opin. Virol. 21:75-80.

It will be appreciated that while the different serotypes can provide some level of cell, tissue, and/or organ selectivity, each serotype still is multi-tropic and thus can result in tissue-toxicity if using that serotype to target a tissue that the serotype is less efficient in transducing. Thus, in addition to achieving some tissue targeting capacity via selecting an AAV of a particular serotype, it will be appreciated that the tropism of the AAV serotype can be modified by an engineered AAV capsid described herein. As described elsewhere herein, variants of wild-type AAV of any serotype can be generated via a method described herein and determined to have a particular cell-selective tropism, which can be the same or different as that of the reference wild-type AAV serotype. In some embodiments, the cell, tissue, and/or selectivity of the wild-type serotype can be enhanced (e.g., made more selective or specific for a particular cell type that the serotype is already biased towards). For example, wild-type AAV-9 is biased towards muscle and brain in humans (see e.g., Srivastava. 2017. Curr. Opin. Virol. 21:75-80.) By including an engineered AAV capsid and/or capsid protein variant of wild-type AAV-9 as described herein, the bias for the brain can be reduced or eliminated and/or the CNS endothelial cell-septicity increased such that the brain selectivity appears reduced in comparison, thus enhancing the selectivity for the muscle as compared to the wild-type AAV-9.

In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. It will be appreciated that wild-type hybrid AAV particles suffer the same selectivity issues as with the non-hybrid wild-type serotypes previously discussed.

Advantages achieved by the wild-type based hybrid AAV systems can be combined with the increased and customizable cell-selectivity that can be achieved with the engineered AAV capsids can be combined by generating a hybrid AAV that can include an engineered AAV capsid described elsewhere herein. It will be appreciated that hybrid AAVs can contain an engineered AAV capsid containing a genome with elements from a different serotype than the reference wild-type serotype that the engineered AAV capsid is a variant of. For example, a hybrid AAV can be produced that includes an engineered AAV capsid that is a variant of an AAV-9 serotype that is used to package a genome that contains components (e.g., rep elements) from an AAV-2 serotype. As with wild-type based hybrid AAVs previously discussed, the tropism of the resulting AAV particle will be that of the engineered AAV capsid.

A tabulation of certain wild-type AAV serotypes as to these cells can be found in Grimm, D. et al, J. Virol. 82: 5887-5911 (2008) reproduced below as Table 1. Further tropism details can be found in Srivastava. 2017. Curr. Opin. Virol. 21:75-80 as previously discussed.

TABLE 1

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

In one example embodiment, the AAV vector or system thereof is AAV rh.74 or AAV rh.10.

In another example embodiment, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the engineered AAV capsid polynucleotide(s)).

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more elements of a engineered AAV capsid system described herein are as used in the foregoing documents, such as WO 2014/093622 (PCT/US2013/074667) and are discussed in greater detail herein.

Virus Particle Production from Viral Vectors

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the engineered AAV capsid polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., the engineered AAV capsid polynucleotide(s)) between 2 ITRs; (2) a vector that carries the AAV Rep-Cap encoding polynucleotides; and helper polynucleotides. One of skill in the art will appreciate various methods and variations thereof that are both helper and helper free and as well as the different advantages of each system.

The engineered AAV vectors and systems thereof described herein can be produced by any of these methods.

A vector (including non-viral carriers) described herein can be introduced into host cells to thereby produce transcripts, proteins, or peptides, including fusion proteins or peptides encoded by nucleic acids as described herein (e.g., engineered AAV capsid system transcripts, proteins, enzymes, mutant forms thereof, fusion proteins thereof, etc.), and virus particles (such as from viral vectors and systems thereof).

One or more engineered AAV capsid polynucleotides can be delivered using adeno associated virus (AAV), adenovirus or other plasmid or viral vector types as previously described, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus.

For plasmid delivery, the route of administration, formulation and dose can be as in U.S. Pat. No. 5,846,946 and as in clinical studies involving plasmids. In some embodiments, doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into or otherwise delivered to the tissue or cell of interest.

In terms of in vivo delivery, AAV is advantageous over other viral vectors for a couple of reasons such as low toxicity (this may be due to the purification method not requiring ultra-centrifugation of cell particles that can activate the immune response) and a low probability of causing insertional mutagenesis because it doesn't integrate into the host genome.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

Delivery of engineered AAV capsid system components (e.g., polynucleotides encoding engineered AAV capsid and/or capsid proteins) to cells via particles. The term “particle” as used herein refers to any suitable sized particles for delivery of the engineered AAV capsid system components described herein. Suitable sizes include macro-, micro-, and nano-sized particles. In some embodiments, any of the of the engineered AAV capsid system components (e.g., polypeptides, polynucleotides, vectors and combinations thereof described herein) can be attached to, coupled to, integrated with, otherwise associated with one or more particles or component thereof as described herein. The particles described herein can then be administered to a cell or organism by an appropriate route and/or technique. In some embodiments, particle delivery can be selected and be advantageous for delivery of the polynucleotide or vector components. It will be appreciated that in embodiments, particle delivery can also be advantageous for other engineered capsid system molecules and formulations described elsewhere herein.

### Engineered Virus Particles Including an Engineered Viral (e.g., AAV) Capsid

Also described herein are engineered virus particles (also referred to here and elsewhere herein as “engineered viral particles”) that can contain an engineered viral capsid (e.g., AAV capsid, referred to as “engineered AAV particles”) as described in detail elsewhere herein. It will be appreciated that the engineered AAV particles can be adenovirus-based particles, helper adenovirus-based particles, AAV-based particles, or hybrid adenovirus-based particles that contain at least one engineered AAV capsid proteins as previously described. An engineered AAV capsid is one that that contains one or more engineered AAV capsid proteins as are described elsewhere herein. The engineered AAV particles can thus include one or more targeting moieties previously described.

The engineered AAV particle can include one or more cargo polynucleotides. Cargo polynucleotides are discussed in greater detail elsewhere herein. Methods of making the engineered AAV particles from viral and non-viral vectors are described elsewhere herein. Formulations containing the engineered virus particles are described elsewhere herein.

### Cargo Polynucleotides

Cargos are also described elsewhere herein. In some embodiments, the cargo is a cargo polynucleotide that can be packaged into an engineered viral particle and subsequently delivered to a cell. In some embodiments, delivery is cell selective, e.g. endothelial cell of the central nervous system vasculature. The engineered viral (e.g., AAV) capsid polynucleotides, other viral (e.g., AAV) polynucleotide(s), and/or vector polynucleotides can contain one or more cargo polynucleotides. In some embodiments, the one or more cargo polynucleotides can be operably linked to the engineered viral (e.g., AAV) capsid polynucleotide(s) and can be part of the engineered viral (e.g., AAV) genome of the viral (e.g., AAV) system of the present invention. The cargo polynucleotides can be packaged into an engineered viral (e.g., AAV) particle, which can be delivered to, e.g., a cell. In some embodiments, the cargo polynucleotide can be capable of modifying a polynucleotide (e.g., gene or transcript) of a cell to which it is delivered. As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA. Polynucleotide, gene, transcript, etc. modification includes all genetic engineering techniques including, but not limited to, gene editing as well as conventional recombinational gene modification techniques (e.g., whole or partial gene insertion, deletion, and mutagenesis (e.g. insertional and deletional mutagenesis) techniques.

In one example embodiments, the cargo molecule is a polynucleotide that is or can encode a vaccine. In another example embodiment, the cargo molecule is a polynucleotide encoding an antibody.

### Gene Modification Cargo Polynucleotides

In some embodiments, the cargo molecule can be a polynucleotide or polypeptide that can alone, or when delivered as part of a system, whether or not delivered with other components of the system, operate to modify the genome, epigenome, and/or transcriptome of a cell to which it is delivered. Such systems include, but are not limited to, CRISPR-Cas systems. Other gene modification systems, e.g., TALENs, Zinc Finger nucleases, Cre-Lox, morpholinos, etc., are other non-limiting examples of gene modification systems whose one or more components can be delivered by the engineered viral (e.g., AAV) particles described herein.

In some embodiments, the cargo molecule is a gene editing system or component thereof. In some embodiments, the cargo molecule is a CRISPR-Cas system molecule or a component thereof. In some embodiments, the cargo molecule is a polynucleotide that encodes one or more components of a gene modification system (such as a CRISPR-Cas system). In some embodiments the cargo molecule is a gRNA. CRISPR-Cas system as used herein is intended to encompass by Class 1 and Class 2 CRISPR-Cas systems and derivatives of CRISPR-Cas systems such as base editors, prime editors, and CRISPR-associated transposases (CAST) systems.

An embodiment of the invention encompasses methods of modifying a genomic locus of interest to change gene expression in a cell by introducing into the cell any of the compositions described herein.

An embodiment of the invention is that the above elements are comprised in a single composition or comprised in individual compositions. These compositions may advantageously be applied to a host to elicit a functional effect on the genomic level.

### Engineered Cells and Organisms Expressing Said Engineered AAV Capsids

Described herein are engineered cells that can include one or more of the engineered AAV capsid polynucleotides, polypeptides, vectors, and/or vector systems. In some embodiments, one or more of the engineered AAV capsid polynucleotides can be expressed in the engineered cells. In some embodiments, the engineered cells can be capable of producing engineered AAV capsid proteins and/or engineered AAV capsid particles that are described elsewhere herein. Also described herein are modified or engineered organisms that can include one or more engineered cells described herein. The engineered cells can be engineered to express a cargo molecule (e.g., a cargo polynucleotide) dependently or independently of an engineered AAV capsid polynucleotide as described elsewhere herein.

A wide variety of animals, plants, algae, fungi, yeast, etc. and animal, plant, algae, fungus, yeast cell or tissue systems may be engineered to express one or more nucleic acid constructs of the engineered AAV capsid system described herein using various transformation methods mentioned elsewhere herein. This can produce organisms that can produce engineered AAV capsid particles, such as for production purposes, engineered AAV capsid design and/or generation, and/or model organisms. In some embodiments, the polynucleotide(s) encoding one or more components of the engineered AAV capsid system described herein can be stably or transiently incorporated into one or more cells of a plant, animal, algae, fungus, and/or yeast or tissue system. In some embodiments, one or more of engineered AAV capsid system polynucleotides are genomically incorporated into one or more cells of a plant, animal, algae, fungus, and/or yeast or tissue system. Further embodiments of the modified organisms and systems are described elsewhere herein. In some embodiments, one or more components of the engineered AAV capsid system described herein are expressed in one or more cells of the plant, animal, algae, fungus, yeast, or tissue systems.

### Engineered Cells

Described herein are various embodiments of engineered cells that can include one or more of the engineered AAV capsid system polynucleotides, polypeptides, vectors, and/or vector systems described elsewhere herein. In some embodiments, the cells can express one or more of the engineered AAV capsid polynucleotides and can produce one or more engineered AAV capsid particles, which are described in greater detail herein. Such cells are also referred to herein as “producer cells”. It will be appreciated that these engineered cells are different from “modified cells” described elsewhere herein in that the modified cells are not necessarily producer cells (i.e., they do not make engineered GTA delivery particles) unless they include one or more of the engineered AAV capsid polynucleotides, engineered AAV capsid vectors or other vectors described herein that render the cells capable of producing an engineered AAV capsid particle. Modified cells can be recipient cells of an engineered AAV capsid particles and can, in some embodiments, be modified by the engineered AAV capsid particle(s) and/or a cargo polynucleotide delivered to the recipient cell. Modified cells are discussed in greater detail elsewhere herein. The term modification can be used in connection with modification of a cell that is not dependent on being a recipient cell. For example, isolated cells can be modified prior to receiving an engineered AAV capsid molecule.

In an embodiment, the invention provides a non-human eukaryotic organism; for example, a multicellular eukaryotic organism, including a eukaryotic host cell containing one or more components of an engineered delivery system described herein according to any of the described embodiments. In other embodiments, the invention provides a eukaryotic organism; preferably a multicellular eukaryotic organism, comprising a eukaryotic host cell containing one or more components of an engineered delivery system described herein according to any of the described embodiments. In some embodiments, the organism is a host of AAV.

In particular embodiments, the plants, algae, fungi, yeast, etc., cells or parts obtained are transgenic plants, comprising an exogenous DNA sequence incorporated into the genome of all or part of the cells.

The engineered cell can be a prokaryotic cell. The prokaryotic cell can be bacterial cell. The prokaryotic cell can be an archaea cell. The bacterial cell can be any suitable bacterial cell. Suitable bacterial cells can be from the genus Escherichia, Bacillus, Lactobacillus, Rhodococcus, Rodhobacter, Synechococcus, Synechoystis, Pseudomonas, Psedoaltermonas, Stenotrophamonas, and Streptomyces Suitable bacterial cells include, but are not limited to Escherichia coli cells, Caulobacter crescentus cells, Rodhobacter sphaeroides cells, Psedoaltermonas haloplanktis cells. Suitable strains of bacterial include, but are not limited to BL21(DE3), DL21(DE3)-pLysS, BL21 Star-pLysS, BL21-SI, BL21-AI, Tuner, Tuner pLysS, Origami, Origami B pLysS, Rosetta, Rosetta pLysS, Rosetta-gami-pLysS, BL21 CodonPlus, AD494, BL2trxB, HMS174, NovaBlue(DE3), BLR, C41(DE3), C43(DE3), Lemo2l(DE3), Shuffle T7, ArcticExpress and ArticExpress (DE3).

The engineered cell can be a eukaryotic cell. The eukaryotic cells may be those of or derived from a particular organism, such as a plant or a mammal, including, but not limited to, human, or non-human eukaryote or animal or mammal as herein discussed, e.g., mouse, rat, rabbit, dog, livestock, or non-human mammal or primate. In some embodiments the engineered cell can be a cell line. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, ClR, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR293, BxPC3, C3H-1OT1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, the engineered cell can be a fungal cell. As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerervisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella 56sabelline).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains can include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific or selective regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In some embodiments, the engineered cell is a cell obtained from a subject. In some embodiments, the subject is a healthy or non-diseased subject. In some embodiments, the subject is a subject with a desired physiological and/or biological characteristic such that when an engineered AAV capsid particle is produced it can package one or more cargo polynucleotides that can be related to the desired physiological and/or biological characteristic and/or capable of modifying the desired physiological and/or biological characteristic. Thus, the cargo polynucleotides of the produced engineered AAV capsid particle can be capable of transferring the desired characteristic to a recipient cell. In some embodiments, the cargo polynucleotides are capable of modifying a polynucleotide of the engineered cell such that the engineered cell has a desired physiological and/or biological characteristic.

In some embodiments, a cell transfected with one or more vectors described herein is used to establish a new cell line comprising one or more vector-derived sequences.

The engineered cells can be used to produce engineered viral (e.g., AAV) capsid polynucleotides, vectors, and/or particles. In some embodiments, the engineered viral (e.g., AAV) capsid polynucleotides, vectors, and/or particles are produced, harvested, and/or delivered to a subject in need thereof. In some embodiments, the engineered cells are delivered to a subject. Other uses for the engineered cells are described elsewhere herein. In some embodiments, the engineered cells can be included in formulations and/or kits described elsewhere herein.

The engineered cells can be stored short-term or long-term for use at a later time. Suitable storage methods are generally known in the art. Further, methods of restoring the stored cells for use (such as thawing, reconstitution, and otherwise stimulating metabolism in the engineered cell after storage) at a later time are also generally known in the art.

### Formulations

The compositions, polynucleotides, polypeptides, particles, cells, vector systems and combinations thereof described herein can be contained in a formulation, such as a pharmaceutical formulation. In some embodiments, the formulations can be used to generate polypeptides and other particles that include one or more selective targeting moieties described herein. In some embodiments, the formulations can be delivered to a subject in need thereof. In some embodiments, component(s) of the engineered AAV capsid system, engineered cells, engineered AAV capsid particles, and/or combinations thereof described herein can be included in a formulation that can be delivered to a subject or a cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, or combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 pg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 pg to about 10 g, from about 10 nL to about 10 ml. In embodiments where the pharmaceutical formulation contains one or more cells, the amount can range from about 1 cell to 1× 10², 1×10³, 1×10⁴, 1×10¹, 1×10⁶, 1×10⁷, 1×10⁸, 1×10⁹, 1×10¹⁰ or more cells. In embodiments where the pharmaceutical formulation contains one or more cells, the amount can range from about 1 cell to 1×10², 1×10³, 1×10⁴, 1×10⁵, 1×10⁶, 1×10⁷, 1×10¹, 1×10⁹, 1×10¹⁰ or more cells per nL, L, mL, or L.

In embodiments, were engineered AAV capsid particles are included in the formulation, the formulation can contain 1 to 1×10¹, 1×10², 1×10³, 1×10⁴, 1×10⁵, 1×10⁶, 1×10⁷, 1×10¹, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles. In some embodiments, the formulation can be 0.1 to 100 mL in volume and can contain 1 to 1×10¹, 1×10², 1× 10³, 1× 10⁴, 1× 10⁵, 1×10⁶, 1× 10⁷, 1× 10⁸, 1× 10⁹, 1×10¹⁰, 1×10¹¹, 1×10¹², 1×10¹3, 1×10¹⁴, 1×10¹¹, 1×10¹⁶, 1×10¹⁷, 1×10¹⁸, 1×10¹⁹, or 1×10²⁰ transducing units (TU)/mL of the engineered AAV capsid particles.

### Pharmaceutically Acceptable Carriers and Auxiliary Ingredients and Agents

In embodiments, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to an amount of one or more of the polypeptides, polynucleotides, vectors, cells, engineered AAV capsid particles, nanoparticles, other delivery particles, and combinations thereof described herein, the pharmaceutical formulation can also include an effective amount of an auxiliary active agent, including but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), and steroid hormones (e.g., estradiol, testosterone, tetrahydro testosterone Cortisol). Suitable immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g., IFN-α, IFN-β, IFN-ε, IFN-K, IFN-ω, and IFN-γ), granulocyte colony-stimulating factor, and imiquimod), chemokines (e.g., CCL3, CCL26 and CXCL7), cytosine phosphate-guanosine, oligodeoxynucleotides, glucans, antibodies, and aptamers).

Suitable antipyretics include, but are not limited to, non-steroidal anti-inflammatories (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Suitable anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotonergic antidepressants (e.g., selective serotonin reuptake inhibitors, tricyclic antidepressants, and monoamine oxidase inhibitors), mebicar, fabomotizole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, bifeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammatories (e.g., ibuprofen, naproxen, ketoprofen, and nimesulide), COX-2 inhibitors (e.g., rofecoxib, celecoxib, and etoricoxib), opioids (e.g., morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

Suitable antispasmodics include, but are not limited to, mebeverine, papaverine, cyclobenzaprine, carisoprodol, orphenadrine, tizanidine, metaxalone, methocarbamol, chlorzoxazone, baclofen, dantrolene, baclofen, tizanidine, and dantrolene. Suitable anti-inflammatories include, but are not limited to, prednisone, non-steroidal anti-inflammatories (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives)

Suitable anti-histamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocetirizine, loratadine, meclizine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, ranitidine, and roxatidine), tritoqualine, catechin, cromoglicate, nedocromil, and p2-adrenergic agonists.

Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, albendazole, thiabendazole, oxamniquine), antifungals (e.g., azole antifungals (e.g., itraconazole, fluconazole, parconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proguanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, abacavir, zidovudine, stavudine, emtricitabine, zalcitabine, telbivudine, simeprevir, boceprevir, telaprevir, lopinavir/ritonavir, boceprevir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, cefazoline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, ceftizoxime, and ceftazidime), glycopeptide antibiotics (e.g., vancomycin, dalbavancin, oritavancin, and telavancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erythromycin, azithromycin, clarithromycin, dirithromycin, and troleandomycin), linezolid, sulfamethoxazole/trimethoprim, rifaximin, chloramphenicol, Fosfomycin, metronidazole, aztreonam, bacitracin, penicillin (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g., lomefloxacin, norfloxacin, ofloxacin, gatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfisoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, Fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, dacarbazine, leuprolide, epirubicin, oxaliplatin, asparaginase, estramustine, cetuximab, vismodegib, asparaginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylate, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine (tioguanine), dabrafenib, erlotinib, bexarotene, temozolomide, thiotepa, thalidomide, Bacillus Calmette-Guerin (BCG), temsirolimus, bendamustine hydrochloride, triptorelin, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

In embodiments where there is an auxiliary active agent contained in the pharmaceutical formulation in addition to the one or more of the polypeptides, polynucleotides, CRISPR-Cas complexes, vectors, cells, virus particles, nanoparticles, other delivery particles, and combinations thereof described herein, amount, such as an effective amount, of the auxiliary active agent will vary depending on the auxiliary active agent. In some embodiments, the amount of the auxiliary active agent ranges from 0.001 micrograms to about 1 milligram. In other embodiments, the amount of the auxiliary active agent ranges from about 0.01 IU to about 1000 IU. In further embodiments, the amount of the auxiliary active agent ranges from 0.001 mL to about 1 mL. In yet other embodiments, the amount of the auxiliary active agent ranges from about 1% w/w to about 50% w/w of the total pharmaceutical formulation. In additional embodiments, the amount of the auxiliary active agent ranges from about 1% v/v to about 50% v/v of the total pharmaceutical formulation. In still other embodiments, the amount of the auxiliary active agent ranges from about 1% w/v to about 50% w/v of the total pharmaceutical formulation.

### Dosage Forms

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, M D, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal, or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, mannitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for parenteral administration and/or adapted for any type of injection (e.g., intravenous, intraperitoneal, subcutaneous, intramuscular, intradermal, intraosseous, epidural, intracardiac, intraarticular, intracavernous, gingival, subgingival, intrathecal, intravitreal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including, but not limited to, sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain anti-oxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. In some embodiments, the predetermined amount of the Such unit doses may therefore be administered once or more than once a day. Such pharmaceutical formulations may be prepared by any of the methods well known in the art.

### Kits

Also described herein are kits that contain one or more of the one or more of the compositions, polypeptides, polynucleotides, vectors, cells, or other components described herein and combinations thereof and pharmaceutical formulations described herein. In embodiments, one or more of the polypeptides, polynucleotides, vectors, cells, and combinations thereof described herein can be presented as a combination kit. As used herein, the terms “combination kit” or “kit of parts” refers to the compounds, or formulations and additional components that are used to package, screen, test, sell, market, deliver, and/or administer the combination of elements or a single element, such as the active ingredient, contained therein. Such additional components include, but are not limited to, packaging, syringes, blister packages, bottles, and the like. The combination kit can contain one or more of the components (e.g., one or more of the one or more of the polypeptides, polynucleotides, vectors, cells, and combinations thereof) or formulation thereof can be provided in a single formulation (e.g., a liquid, lyophilized powder, etc.), or in separate formulations. The separate components or formulations can be contained in a single package or in separate packages within the kit. The kit can also include instructions in a tangible medium of expression that can contain information and/or directions regarding the content of the components and/or formulations contained therein, safety information regarding the content of the components(s) and/or formulation(s) contained therein, information regarding the amounts, dosages, indications for use, screening methods, component design recommendations and/or information, recommended treatment regimen(s) for the components(s) and/or formulations contained therein. As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory drive or CD-ROM or on a server that can be accessed by a user via, e.g., a web interface.

In one embodiment, the invention provides a kit comprising one or more of the components described herein. In some embodiments, the kit comprises a vector system and instructions for using the kit. In some embodiments, the vector system includes a regulatory element operably linked to one or more engineered polynucleotides, such as those containing a selective targeting moiety, as described elsewhere herein and, optionally, a cargo molecule, which can optionally be operably linked to a regulatory element. The one or more engineered polynucleotides such as those containing a selective targeting moiety, as described elsewhere herein and, can be included on the same or different vectors as the cargo molecule in embodiments containing a cargo molecule within the kit.

### Methods of Use for Delivery of Cargo to Endothelial Cells of CNS Vasculature

The compositions including one or more of the cell-selective targeting moieties, engineered AAV capsid system polynucleotides, polypeptides, vector(s), engineered cells, engineered AAV capsid particles can be used generally to package and/or deliver one or more cargos to a endothelial cells of the CNS vasculature. In some embodiments, delivery is done in cell-selective manner based upon the selectivity of the targeting moiety. In some embodiments this is conferred by the tropism of the engineered AAV capsid, which can be influenced at least in part by the inclusion of one or n-mer motifs described elsewhere herein. In some embodiments, compositions including one or more of the CNS endothelial targeting moieties, engineered AAV capsid particles, can be administered to a subject or a cell, tissue, and/or organ and facilitate the transfer and/or integration of the cargo to the recipient cell. In other embodiments, engineered cells capable of producing compositions, such as polypeptides and other particles (e.g., engineered AAV capsids and viral particles), containing one or more of the targeting moieties can be generated from the polynucleotides, vectors, and vector systems etc., described herein. This includes without limitation, the engineered AAV capsid system molecules (e.g., polynucleotides, vectors, and vector systems, etc.). In some embodiments, the polynucleotides, vectors, and vector systems etc., described herein capable of generating the compositions, such as polypeptides and other particles (e.g., engineered AAV capsids and viral particles), containing one or more of the targeting moieties can be delivered to a cell or tissue, in vivo, ex vivo, or in vitro. In some embodiments, when delivered to a subject, the composition can transform a subject's cell in vivo or ex vivo to produce an engineered cell that can be capable of making a composition described herein that contains one or more of the cell-selective targeting moieties described herein, including, but not limited to, the engineered AAV capsid particles, which can be released from the engineered cell and deliver cargo molecule(s) to a recipient cell in vivo or produce personalized engineered compositions (e.g., AAV capsid particles) for reintroduction into the subject from which the recipient cell was obtained.

In some embodiments, an engineered cell can be delivered to a subject, where it can release produced compositions of the present invention (including but not limited to engineered AAV capsid particles) such that they can then deliver a cargo (e.g., a cargo polynucleotide(s)) to a recipient cell. These general processes can be used in a variety of ways to treat and/or prevent disease or a symptom thereof in a subject, generate model cells, generate modified organisms, provide cell selection and screening assays, in bioproduction, and in other various applications.

In some embodiments, the compositions, such as polypeptides and other particles (e.g., engineered AAV capsids and viral particles), containing one or more of the targeting moieties can be delivered to endothelial cells of the CNS vasculature.

In some embodiments, the engineered AAV capsid polynucleotides, vectors, and systems thereof can be used to generate engineered AAV capsid variant libraries that can be mined for variants with a desired cell-selectivity. The description provided herein as supported by the various Examples can demonstrate that one having a desired cell-selectivity in mind could utilize the present invention as described herein to obtain a capsid with the desired cell-selectivity.

As discussed above, the targeting moieties of the present invention confer a strong tropism bias for across the arterio-venous axis in brain, retina, and spinal cord vasculature, including arterial, capillary, and venous endothelial cells. However, in certain context, transduction may also occur to a lesser extent in liver hepatocyte, lung microvascular endothelial cells, and the endothelial lining of large arteries and veins through the systemic circulation following intravenous administration. When deployed for research purposes where CNS-selectivity is critical, a Cre-dependent viral genome could be used in tandem with a CNS endothelial cell-selective transgenic driver—such as MFSD2A:Cre^{ERT242} or SLCO1C1:Cre^{ERT2 42} to minimize peripheral transduction. For therapeutic uses, one or more repeat elements may be incorporated in the viral vector systems disclosed herein to reduce non-CNS endothelial vasculature expression. For example, to reduce expression in hepatocytes, repeats of the hepatocyte-selective miR-122 target sequence into the 3′UTR.

### Methods of Treatment

Provided herein are methods for treating a disease or disorder, the method comprising administering to a subject in need thereof, either a composition as disclosed herein to vascular endothelial cells of the CNS. In an aspect, the compositions used in methods disclosed herein are capable of crossing the CNS vasculature, allowing for delivery of cargo and therapeutics into or across the blood-brain-barrier. In embodiments, a method is disclosed wherein the cargo is one or more polypeptides.

### Diseases or Disorders

In embodiments, a method is disclosed wherein the disease or disorder is a lysosomal storage disorder, cancer, neurological disorder or infection. In embodiments, a method is disclosed wherein the subject suffers from a lysosomal storage disease and the composition or vector is configured to deliver an enzyme missing in the lysosomal storage disease, or therapeutic polynucleotide encoding the enzyme, to endothelial cells of the CNS vasculature. In embodiments, a method is disclosed wherein the lysosomal storage disease is Fabry disease, MPS II, Krabbe Disease, or Tay-Sachs.

Lysosomal storage disorders can include mucopolysacchridoses (MPS), such as MPS I, MPS II, MPS IIIA, IIIB, IIIB, or HID, MPSIVA, MPS IVB, MPS VI, or MPS VII. The lysosomal storage disease can be glycoproteinoses, e.g., aspartylglycosaminuria, fucoidosis, alpha-manosidosis, beta-Mannosidosis, mucolipidosis I (sialidosis) or Schindler disease. Sphinogolipidoses, e.g., Fabry's disease, Farber's disease, Gaucher's disease, GM1 gangliosidosis, Tay-Sachs disease, Sandhoff's disease, Krabbe's disease, Meachromatic leucodystrophy, Niemann-Pick disease, types A and B are further exemplary lysosomal storage diseases for which the compositions and methods herein can be used. Other lipidoses, including Niemann-Pcik disease type C, Wolman's disease, Neuroanal ceroid lipofuscinosis; Glycogen storage disease such as Glycogen storage disease type II (Pompe's disease); Multiple enzyme deficiency, such as multiple sulphatase deficiency, galactosialidosis, mucolipidosis II/III, and mucolipidosis IV; lysosomal transport defects, for example cystinosis, sialic acid storage disease; and other disorders dues to defects in lysosomal proteins such as Danon disease and hyaluronidase deficiency are further examples of lysosomal storage diseases that can be treated in accordance with the methods and compositions described herein. Further lysosomal storage diseases are described in Table 1 of Platt et al., (2012) J. Cell. Biol. Col. 199, no. 5 723-734, incorporated herein by reference. Methods as detailed herein may be used with additional therapies for lysosomal storage diseases, exemplary therapies are described in Table 2 of Platt et al, incorporated herein by reference.

In an embodiment, methods of treatment comprise administering a composition as detailed herein to a subject in need thereof. In one embodiment, the cancer is a neuroepithelial cancer. In an embodiment, the cancer is a neuroepithelial tumor, for example, Astrocytic tumors, e.g., Diffuse Astrocytoma (fibrillary, protoplasmic, gemistocytic, mixed), Anaplastic (malignant) astrocytoma, Glioblastoma (giant cell, gliosarcoma variants), Pilocytic astrocytoma, Pleomorphic xanthoastrocytoma, or Subependymal giant cell astrocytoma; Oligodendroglial tumors, e.g., Oligodendroglioma, Anaplastic (malignant) Oligodendroglioma, Ependymal tumors, Ependymoma (cellular, papillary, clear cell, tanycytic), Anaplastic (malignant) ependymoma, ependymoma, Subependymoma; Mixed tumors, e.g., Oligoastrocytomaor Anaplastic (malignant) oligoastrocytoma; Choroid plexus tumors, e.g., Choroid Plexus papilloma or Choroid Plexus carcinoma; Neuronal and mixed neuronal-glial tumors, e.g. Gangliocytoma, Gangloglioma, Dysembryoplastic neuroepithelial tumor (DNET), Dysplastic gangliocytoma of the cerebellum (Lhermitte-Duclos), Desmoplastic infantile astrocytoma/ganglioglioma, Central neurocytoma, Anaplastic ganglioglioma, Cerebellar liponeurocytoma, Paraganglioma of the filum terminale; Pineal tumors, e.g., Pineocytoma, Pineoblastoma, Pineal parenchymal tumor of intermediate differentiation; Embryonal tumors, e.g., Medulloblastoma (desmoplastic, large cell, melanotic, medullomyoblastoma), Medulloepithelioma, Supratentorial primitive neuroectodermal tumors, PNETs such as Neuroblastoma, Ganglioneuroblastoma, Ependymoblastoma, or Atypical teratoid/rhabdoid tumor; Neuroblastic tumors, e.g., Olfactory (esthesioneuroblastoma), Olfactory neuroepithelioma, Neuroblastomas of the adrenal gland and sympathetic nervous system; Glial tumors of uncertain etiology, e.g., Astroblastoma, Gliomatosis cerebri, Chordoid glioma of the third ventricle.

In an embodiment, the cancer is a primary cancer metastasized to brain, central nervous system, hepatocytes or vascular endothelial cells.

In embodiments, a method is disclosed, wherein the subject suffers from hemophilia A, and the composition or vector is configured to deliver a truncated Factor VIII, or polynucleotide encoding a truncated Factor VIII, to vascular endothelial cells.

Metabolic disorders of the brain that manifest in the neonatal or early infantile period are usually associated with acute and severe illness and are thus referred to as devastating metabolic disorders. Most of these disorders may be classified as organic acid disorders, amino acid metabolism disorders, primary lactic acidosis, fatty acid oxidation disorders and nutrient transport disorders. Each disorder has distinctive clinical, biochemical, and radiologic features. Early diagnosis is important both for prompt treatment to prevent death or serious sequelae and for genetic counseling. However, diagnosis is often challenging because many findings overlap and may mimic those of more common neonatal conditions, such as hypoxic-ischemic encephalopathy and infection. If one of these rare disorders is suspected, the appropriate biochemical test or analysis of the specific gene should be performed to confirm the diagnosis.

One such nutrient transport disorder affecting brain development and function is GLUT1 deficiency, also known as GLUT1 Deficiency Syndrome, GLUT1-DS, De Vivo Disease or Glucose Transporter Type 1 Deficiency Syndrome. GLUT1-DS is an autosomal dominant, genetic metabolic disorder associated with a deficiency of wild-type, fully functioning GLUT1, the protein that transports glucose across the blood brain barrier. The (GLUT1 protein that transports glucose across the blood brain barrier is made by the SLC2A1 gene, located on chromosome 1. In GLUT1 Deficiency Syndrome, one of the two SLC2AI genes is damaged by a mutation and as a result insufficient GLUT1 protein is made. As a result of a deficiency of wild-type levels of fully functional GLUT1 transporter protein, insufficient glucose passes the blood brain barrier. Having less functional GLUT1 protein reduces the amount of glucose available to brain cells, which affects brain development and function. Because glucose is the primary source of fuel for the brain, patients with GLUT1 deficiency have insufficient cellular energy to permit normal brain growth and function (Reaching for a Brighter Future, Pamphlet of the GLUT1 Deficiency Foundation, Oct. 2, 2015). Around 90% of cases of GLUT1 deficiency syndrome are de novo mutations of the SLC2A1 gene, i.e., a mutation is not present in the parents, but present in one of the two copies of the gene in the baby. GLUT1 Deficiency can also be inherited in an autosomal dominant manner. A person with GLUT1 deficiency syndrome has a 50% chance of passing along the altered SLC2A1 gene to his or her offspring.

### SLC2A1 Sequence

SLC2A1, also known as CSE, DYT17, DYT18, DYT9, EIG12, GLUT, GLUT-1, GLUT1, GLUT1DS, HTLVR, PED, SDCHCN, is located on the human chromosome 1, at the 1p34.2 locus. In one example embodiment, the polynucleotide sequence included in the AAV vector is a DNA sequence derived from the primary accession number NG_008232.1. In another example embodiment, the DNA sequence is NG_008232.1. In another example embodiment, the DNA sequence is derived from the secondary accession numbers A8K9S6, B2R620, D3DPX0, 075535, Q0P512 AND Q147X2. In another example embodiment, the DNA sequence is selected from the group consisting of A8K9S6, B2R620, D3DPX0, 075535, Q0P512 AND Q147X2. In another example embodiment, the SLC2A1 gene is derived from a genomic sequence with accession numbers AC99795.2, CH471059.2, CQ918450.1, M20653.1, MW883607.1 and MW883608.1. In another example embodiment, the SLC2A1 genomic sequence is selected from the group consisting of AC99795.2, CH471059.2, CQ918450.1, M20653.1, MW883607.1 and MW883608.1.

In another example embodiment, the polynucleotide sequence included in the AAV vector is a RNA sequence derived from NM_006516. In another example embodiment, the polynucleotide sequence included in the AAV vector is NM_006516. In another example embodiment, the sequence included in the AAV vector is derived from mRNA with the accession numbers: AB208987.1; AF070544.1; AK122999.1; AK292791.1; AK293306.1; AK296736.1; AK312403.1; AW137914.1; AY034633.1; BC118590.1; BC121804.1; BG682043.1; BI490999.1; BP314853.1; BQ948542.1; DA753077.1; and K03195.1. In another example embodiment, the sequence included in the vector is a mRNA sequence selected from the group consisting of: AB208987.1; AF070544.1; AK122999.1; AK292791.1; AK293306.1; AK296736.1; AK312403.1; AW137914.1; AY034633.1; BC118590.1; BC121804.1; BG682043.1; BI490999.1; BP314853.1; BQ948542.1; DA753077.1; and K03195.1. In another example embodiment, the GLUT1 protein sequence is derived from the primary accession number P11166.2, and NP_006507.2. In another example embodiment, the GLUT1 protein sequence is derived from the protein sequence with accession numbers: EAX07123.1; EAX07124.1; CAI23886.1; AAB61084.1; QTW97776.1; QTW97777.1; BAD92224.1; AAC28635.1; BAG53842.1; BAF85480.1; BAG56827.1; BAG59323.1; BAG35317.1; AAK56795.1; AAI8591.1; AAI21805.1 and AAA52571.1. In another example embodiment, the GLUT1 protein sequence is selected from the group consisting of: EAX07123.1; EAX07124.1; CAI23886.1; AAB61084.1; QTW97776.1; QTW97777.1; BAD92224.1; AAC28635.1; BAG53842.1; BAF85480.1; BAG56827.1; BAG59323.1; BAG35317.1; AAK56795.1; AAI8591.1; AAI21805.1 and AAA52571.1. In another example embodiment, the GLUT1 protein sequence is derived from the secondary accession numbers A8K9S6, B2R620, D3DPX0, 075535, Q0P512 and Q147X2. In another example embodiment, the GLUT1 protein sequence is selected from the group consisting of: A8K9S6, B2R620, D3DPX0, 075535, Q0P512 and Q147X2.

In embodiments, a method is disclosed wherein the subject suffers from a GLUT1 deficiency, and the compositions or vectors described herein are configured to deliver a wild-type cargo of GLUT1, or a polynucleotide encoding GLUT1 (i.e., a wild-type cargo of SLC2A1), to vascular endothelial cells of the CNS.

### Gene Delivery Approaches for Increasing Wild-Type SLC2A1 and GLA Expression

In one example embodiment, subjects at risk for, or suffering from, a Glut1 deficiency or Fabry disease are treated by delivering a cargo using the compositions as described herein and/or the vector systems as described herein of the wild-type SLC2A1 gene (to treat Glut1 deficiency) or wild-type GLA gene (to treat Fabry disease) to endothelial cells, i.e., increasing expression of a wild-type copy of the SLC2A1 gene or a wild-type copy of the GLA gene to restore normal levels of these critical gene products in the vascular endothelial cells using a gene therapy approach. As used herein, the terms “gene therapy” and “gene delivery” are used interchangeably and refer to modifying or manipulating the expression level of a gene to alter the biological properties of living cells for therapeutic use.

In another example embodiment, gene therapy approaches may be used to deliver endothelial cells to produce and secrete gene products such as clotting factors, for example Factor VIII, for treating hemophilia A, or lysosomal enzymes or antibodies, for treating lysosomal storage diseases.

Further embodiments are illustrated in the following Examples which are given for illustrative purposes only and are not intended to limit the scope of the invention.

### EXAMPLES

### Example 1

To date, only two viral vectors with the ability to efficiently transduce cerebrovascular endothelial cells have been described, AAV-PHP.V1 (Kumar, S. R. et al. (2020), Nat Methods 17, 541-550) and AAV2-BR1 (Körbelin, J. et al. (2016), EMBO Mol. Med. 8, 609-625), both of which have caveats that restrict their use. Within the brain, AAV-PHP.V1 does not selectively transduce endothelial cells; it infects astrocytes with similarly high efficiency. Because astrocytes are intimately associated with the brain vasculature, this lack of specificity limits the vector's utility. AAV2-BR1, by contrast, transduces brain microvascular endothelial cells with high specificity and has been successfully leveraged by a number of groups since its initial discovery (Tan, C. et al. (2019), Neuron 101, 920-937.e13; Santisteban, M. M. et al. (2020), Hypertension 76, 795-807; Liu, X. et al. (2020), J Exp Med 217; Dogbevia, G., et al. (2020), J Cereb Blood Flow Metab 40, 1338-1350; Chen, D. Y. et al. (2021), J Clin Invest 131, e135296 (2021). Nikolakopoulou, A. M. et al. J Exp Med 218, e20202207; Cui, Y. et al. (2021), Cell Rep. 36, 109327; Song. X. et al. (2022), Int J Biol Sci 18, 652-660). While it is well-established that AAV2-BR1 efficiently transduces capillary endothelial cells, it is unclear whether the vector robustly targets the endothelium of arteries and veins. A recent study found the virus' efficacy is diminished in these larger vessel segments (Santisteban, M. M. et al. (2020), Hypertension 76, 795-807), suggesting that AAV2-BR1 is not well-suited to address rapidly emerging interest in the specialized functions of arterial and venous endothelial cells. Moreover, the vector's ability to effectively target spinal cord and retinal vasculature is ambiguous. Initial characterization of AAV2-BR1 revealed that its transduction of the spinal cord endothelium was significantly less efficient than that observed in brain (Körbelin, J. et al. (2016), EMBO Mol. Med. 8, 609-625) and while there is some evidence that the vector transduces endothelial cells of the retina vasculature, its performance in this context remains poorly characterized (Chen, D. Y. et al. (2021), J Clin Invest 131, e135296).

Here, Applicants describe a novel viral capsid that meets the need for a specific, high-efficiency vector to target endothelial cells throughout the entire CNS: AAV-BI30, an engineered variant of AAV9 identified by screening a capsid library for variants with improved brain transduction in BALB/cJ and C57BL/6J mice and improved transduction of human cells in vitro. At relatively low systemic doses in adult mice, this capsid variant transduces the majority of endothelial cells across the arterio-venous axis in brain, retina, and spinal cord vasculature. Furthermore, the capsid's transduction profile extends across species: Applicants observed robust endothelial transduction in C57BL/6 and BALB/cJ mouse strains, in rats, and in mouse and human brain microvascular endothelial cells (BMVECs). Taken together, these attributes make AAV-BI30 exceptionally well-suited to accelerate our understanding of neurovascular interactions in normal physiology and pioneer therapies to address their dysfunction in disease.

### Results

The ability to target gene delivery to the vasculature of the brain, and central nervous system more broadly, could enable novel therapeutic strategies for the treatment of a variety of neurological disorders, lysosomal storage disorders, cancers, and infections. For some indications, for example Glut1 deficiency or Fabry disease, delivering a copy of the SLC2A1 gene (Glut1) or GLA gene (Fabry) to endothelial cells is a promising therapeutic strategy to restore normal levels of the critical gene products in the vascular endothelial cells. In other examples, endothelial cells could be used to produce and secrete gene products such as clotting factors such as Factor VIII, or lysosomal enzymes or antibodies that then affect additional cell types in the central nervous system. Our invention is a family of engineered AAV capsids that are enriched by selection for more efficient brain transduction and in vitro endothelial cell transduction across multiple species. Several of these sequences show more specific and efficient brain endothelial transduction in vivo, and one variant, AAV-BI30, which Applicants have characterized extensively, efficiently transduces endothelial cells of the brain, spinal cord, and retina after intravenous injection. The AAV-BI30 capsid also enables transduction of endothelial cells in the kidney, lungs, and to a lesser extent endothelial cells of the liver and muscle, as well as highly efficient transduction of liver hepatocytes in multiple mouse strains. Importantly, and in contrast with several prior engineered AAV capsids, the enhanced tropism of AAV-B130 for CNS endothelial cells is present in multiple species including rats and in human and mouse brain microvascular endothelial cells in vitro. Likewise, Applicants have detected that AAV capsids defined by the XNXX[K/R]XX (N2KR5) motif that is shared by AAV-B130 and numerous other AAV variants Applicants have identified (Tables 1-6) are enriched in mice, human and mouse brain endothelial cells in vitro, and in common marmosets, a species of new world primates. These results suggest that AAV-B1I30, and other members of the N2KR5 family, could be useful for delivering gene therapies to the CNS, lung, and kidney vasculature and/or liver hepatocytes.

### Development of the BI30 Variants

Applicants sought to engineer AAV capsids that would enable gene delivery to endothelial cells throughout the central nervous system in multiple species. To develop variants with this property, libraries were generated of AAV9 (K449R) modified with a linear stretch of 7 amino acids (7-mer) inserted between residues 588-589 (VP1 position) as previously described in International Patent Publication WO 2020160337 at Example 1 at pages 57-58 of the published application, specifically incorporated herein by reference. The library was cloned into a recombinant AAV genome designed to express the AAV capsid during virus production and in transduced cells, thereby enabling the selective recovery of functional capsids by reading out capsid mRNA regardless of the cell type or species. The vector contains the full length AAV9 K449R capsid gene driven by a hybrid AAV5 p41-AAV2 3′ rep gene sequence, which contains a splice donor. To enhance expression in transduced cells an additional promoter sequence upstream of the AAV5 p41 promoter was inserted. Two versions of the construct in this study were used: one with a ubiquitous CMV enhancer-promoter (CMV-AAV-Express) and another with a human Synapsin 1 (hSyn) promoter (hSyn-AAV-Express), designed to enhance expression more selectively in transduced neurons, see, e.g. International Patent Publication WO 2020160337. The libraries were screened using assays designed to read out two different stages of virus function (binding/biodistribution and transduction). Binding and biodistribution were assessed by applying the library to specific cell types or administering the virus to mice by intravenous injection. 1-4 hours later, AAV capsid variants that remained associated with the cells or brain tissue were amplified by PCR using primers that flanked the 7-mer variable region and quantified by NGS. To assess transduction, cellular or tissue RNA was isolated and converted to cDNA prior to PCR amplification and quantification by NGS. The AAV9 K449R 7-mer libraries was assessed through several screening assays to identify capsids with more efficient binding and transduction of brain cells in vivo (C57BL/6J and BALB/cJ mice and marmoset) and human and mouse endothelial cells in vitro (human and mouse primary brain microvascular endothelial cells (hBMVEC and mBMVEC) and human CMEC/D3 brain endothelial cells).

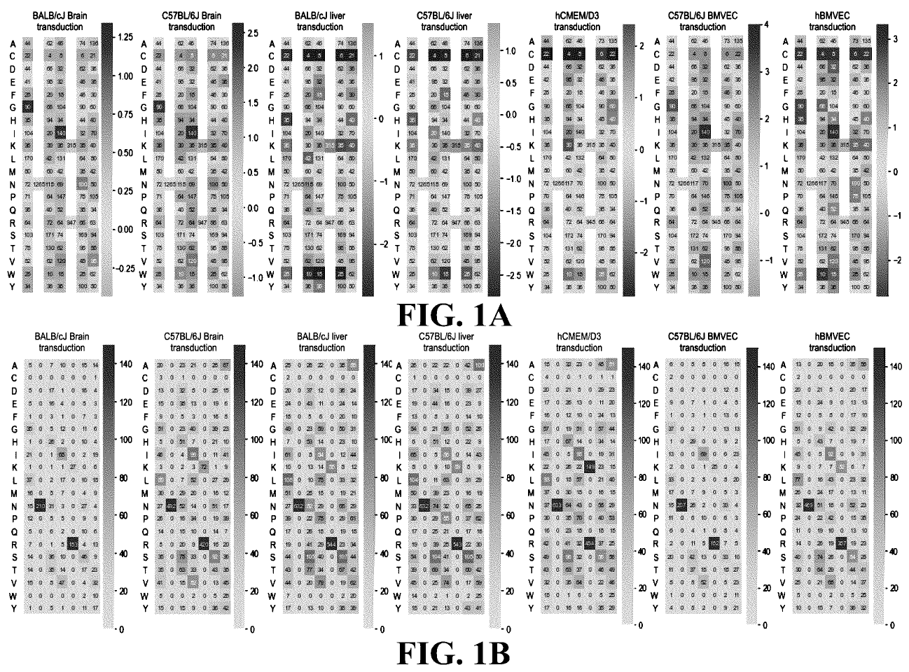

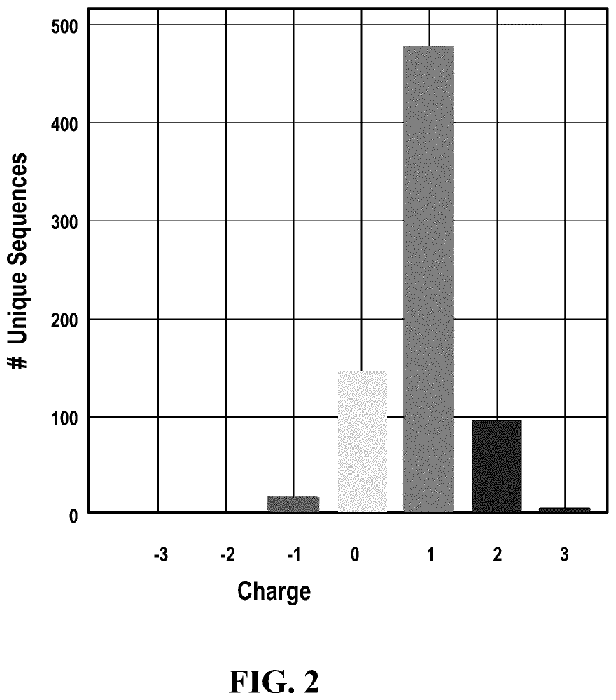

Through these assays a family of variants was identified with a distinct sequence motif that was enriched regardless of mouse strain or species. The sequence motif is most broadly defined as XNXX[K/R]XX, where X is any of the 20 amino acids and position 2 is N and position 4 is a positively charged residue K/R. Several other features define the most enriched sequences within this motif, including a strong preference for T/V/I at position 4 XNX[T/V/I][K/R]XX, or occasionally a A/M/S at position 4 XNX[T/V/I/A/M/S][K/R]XX. The most enriched sequences recovered from several screening assays are provided in Table 1-6. To assess the contribution of each amino acid at each position within this family, the average enrichment for variants with a specific amino acid at a specific position were plotted on heat maps (FIG. 1A-1B). Applicants also assessed the overall charge distribution of the enriched variant examples provided in Tables 1-6. The vast majority of enriched 7-mer variants within the XNXX[K/R]XX family have an overall charge of 0 or +1, with a lesser number of variants with an overall charge change of +2 (FIG. 2).

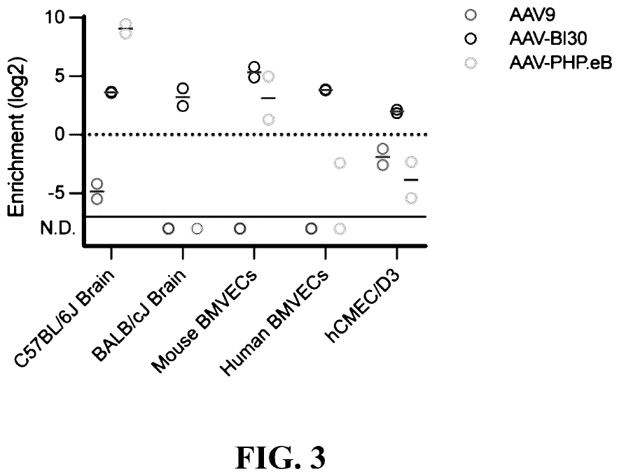

FIG. 3 Enrichment of AAV-B130 by in vivo and in vitro selection. An AAV9 7-mer library was intravenously administered to (i) adult C57BL/6J and BALB/cJ mice at 1×10¹¹ vg/animal and (ii) human & mouse primary BMVECs and hCMEC/D3 human endothelial cells in vitro at 1×10⁴ vg/cell. Capsid mRNA was recovered from mouse brain or from cells in vitro after 21 or 3 days, respectively. The enrichment of AAV-B130 as well as AAV9 and AAV-PHIP.eB controls was calculated as the log 2 of the variant reads per million (RPM) in the indicated assay divided by the variant RPM in the virus library. Each of the three variants was represented by two distinct nucleotide sequences: replicate sequences (circles) are shown along with the mean. N.D. indicates sequences not detected in the assay.

### 7-Mer Linear Insertion N2KR5 Motif Description

In an example embodiment the composition of the targeting moiety is in AAV9 between positions 588Q (Gln) and 589A (Ala), and is exemplified by X1-X2-X3-X4-X5-X6-X7, where each X represents an amino acid. In an embodiment, Position X1 is selected from the group consisting of amino acids G, M, T, S, N, D, L, H, P, I, V, Q, Y, W, F, A, E. Position X3 is selected from the group consisting of amino acids N, S, T, H, D, A, Y, M, Q, E, R, G, V. Position X4 is selected from the group consisting of T, V, I, A, M, S, H, W, N. Position X5 is selected from R or K. Position X6 is selected from the group consisting of N, S, G, D, P, T, H, Q, A, Y. Position X7 is selected from the group consisting of T, Y, W, N, V, I, H, M, S, G, A, Q, F, D, P, R, L.

In an example embodiment, the composition of X1, X3, X4, X6, and X7 are independently selected from the following groups. Position X1 is selected from the group consisting of G, M, T, S, N, D. Position X3 is selected from the group consisting of N, S, T, H, D. Position X4 is selected from the group consisting of T, V, I, A. Position X6 is selected from the group consisting of N, S, G, D, P. Position X7 is selected from the group consisting of T, Y, W, N, V, I, H, M, S, G, A, Q, F, D, P, R, L.

In an example embodiment, the composition of the n-mer at position X1 is R or K and X3, X4, X6 and X7 are D or E.

In an example embodiment, the composition of the n-mer at position X1 is not R, K or C; X3 is not W, F, K, C, I, P, or L; X4 is not Y, G, P, D, C, Q, R, K, E, F, L, or R; X6 is not R, I, W, V, F, C, L, E, or K; or X7 is not C, K, E.

The overall charge of the 7-mer sequence is between 0 and +2

X1-N-X3-X4-[K/R]-X6-X7

X1-X2-N-X3-[T/V/I/A/M/S/H/W/N]-[K/R]-X6-X7

X1-X2-N-X3-[T/V/I/A]-[K/R]-X6-X7

X1-X2-N-X3-[T/V/I/A]-[K/R]-X6-X7, where the overall charge of the 7-mer at neutral pH is between 0 and +2.

[G/M/T/S/N/D]-N-[N/S/T/H/D]-[T/V/I/A]-[K/R]-[N/S/G/D/P]-[T/Y/W/N/V/I/H/M/S/G/A/Q/F/D/P/R/L], where the overall charge of the 7-mer at neutral pH is between 0 and +2.

Specific examples of validated sequences (BI30: NNSTRGG (SEQ ID NO: 1), BI31: GNSARNI (SEQ ID NO: 2), and BI55: GNSVRDF (SEQ ID NO: 3)).

TABLE 2
N2KR5 motif containing AAV9 7-mer variants enriched through an in vivo brain
biodistribution screen in C57BL/6J mice using CMV-AAV-Express

| SEQ | | SEQ | | SEQ | | SEQ | |
|---|---|---|---|---|---|---|---|
| ID NO | AA sequence | ID NO | AA sequence | ID NO | AA sequence | ID NO | AA sequence |
| 6 | ANAAKNY | 7 | FNGIKEM | 8 | NNHTREV | 9 | TNKIRDP |
| 10 | ANALKNQ | 11 | FNKDKED | 12 | NNMIRAV | 13 | TNNTRQN |
| 14 | ANATRLQ | 15 | FNSTRPD | 16 | NNNTKNI | 17 | TNNTRVG |
| 18 | ANATRYQ | 19 | GNATKAG | 20 | NNNTRNH | 21 | TNNVKSG |
| 22 | ANEYKAH | 23 | GNDVKDI | 24 | NNQTRMM | 25 | TNPDRSA |
| 26 | ANGTKNG | 27 | GNETRYT | 28 | NNSDKVK | 29 | TNQTKVH |
| 30 | ANIVKLQ | 31 | GNFVKSG | 32 | NNSIKSQ | 33 | TNSAKPY |
| 34 | ANLAKVM | 35 | GNHSKIQ | 36 | NNSTRTA | 37 | TNSTKGA |
| 38 | ANLVKTT | 39 | GNIIRVH | 40 | NNTIKNG | 41 | TNSTRIG |
| 42 | ANPNKSV | 43 | GNLPKIA | 44 | NNVVKNI | 45 | TNSVKAH |
| 46 | ANSERFL | 47 | GNNIRTQ | 48 | NNYTKHQ | 49 | TNTLKNV |
| 50 | ANSIRDV | 51 | GNNSKPP | 52 | NNYTRPI | 53 | TNTTRSP |
| 54 | ANSTRTW | 55 | GNNTKMG | 56 | PNALRMV | 57 | VNAIRDY |
| 58 | ANTTKIE | 59 | GNPEKND | 60 | PNEQKVI | 61 | VNATRVY |
| 62 | ANVVKQF | 63 | GNQTKNG | 64 | PNIIKQN | 65 | VNGGKFM |
| 66 | CNEGRSC | 67 | GNQTRGG | 68 | PNLSKER | 69 | VNITKNF |
| 70 | DNDHRDI | 71 | GNQVKGG | 72 | PNMQKQI | 73 | VNITKSW |
| 74 | DNDSKSS | 75 | GNRSKDY | 76 | PNSDKLK | 77 | VNMTKPI |
| 78 | DNDVKGG | 79 | GNSVKNG | 80 | PNVAKQQ | 81 | VNMVRTF |
| 82 | DNGTKTF | 83 | GNTIRQA | 84 | QNALRNI | 85 | VNQVKNH |
| 86 | DNGVRGY | 87 | GNTVKGS | 88 | QNEVRNG | 89 | VNVERKL |
| 90 | DNHIKPS | 91 | GNVVKST | 92 | QNFVKIS | 93 | VNVGKGP |
| 94 | DNIFKEF | 95 | HNANKML | 96 | QNLGKFI | 97 | WNGNRAA |
| 98 | DNKIKST | 99 | HNEPKNH | 100 | QNMDKPA | 101 | YNATRGG |
| 102 | DNKLRPI | 103 | HNLDKTH | 104 | QNMVRGN | 105 | YNSTRNG |
| 106 | DNMIRKV | 107 | HNLNKFT | 108 | QNQTRIQ | 109 | YNTQKEI |
| 110 | DNPSRSV | 111 | HNPIRFP | 112 | QNTIRMY | 113 | YNTTKDK |
| 114 | DNQMKFV | 115 | INKEKFT | 116 | QNTTKAG | | |
| 117 | DNTERIP | 118 | INSDKSL | 119 | QNYTKNN | | |
| 120 | DNTERPA | 121 | KNDLRNE | 122 | RNDTKGG | | |
| 123 | DNTNKEE | 124 | KNEPKNL | 125 | SNAIKLS | | |
| 126 | ENADKGR | 127 | KNEVKNA | 128 | SNAPRIV | | |
| 129 | ENDPKAV | 130 | LNAMRPL | 131 | SNAPRQV | | |
| 132 | ENGLKYP | 133 | LNGERQI | 134 | SNAVKSA | | |
| 135 | ENHVKGQ | 136 | LNKDKLI | 137 | SNDIRNN | | |
| 138 | ENKERDV | 139 | LNLNRLE | 140 | SNDKRPL | | |
| 141 | ENKIKLV | 142 | MNGSKTA | 143 | SNHIKSA | | |
| 144 | ENLERLK | 145 | MNQQKLV | 146 | SNHTKNF | | |
| 147 | ENNVKAV | 148 | MNQTRGG | 149 | SNLQKFI | | |
| 150 | ENPIKST | 151 | MNSTKNG | 152 | SNLQKVE | | |
| 153 | ENQIRSL | 154 | MNSVRFM | 155 | SNQVRAL | | |
| 156 | ENQVKQY | 157 | MNVTKQT | 158 | SNSTKSA | | |
| 159 | ENQVRME | 160 | NNATRAA | 161 | SNSTRAP | | |
| 162 | ENQYKAI | 163 | NNATRPA | 164 | SNTTRPI | | |
| 165 | ENTSRTQ | 166 | NNGTRLS | 167 | SNVNRLV | | |
| 168 | ENVERLK | 169 | NNHDKSK | 170 | SNVTKWV | | |
| 171 | ENVIRSV | 172 | NNHIRET | 173 | TNATKGG | | |
| 174 | FNAERMT | 175 | NNHIRYH | 176 | TNDDKYQ | | |
| 177 | FNDAKGT | 178 | NNHTKDI | 179 | TNDVRRL | | |
| 180 | FNDAKQW | 181 | NNHTKGV | 182 | TNEDKGL | | |

Table 2. N2KR5 motif containing AAV9 7-mer variants enriched through an in vivo brain biodistribution screen in C57BL/6J mice using CMV-AAV-Express SEQ ID AA sequence SEQ ID AA sequence SEQ ID AA sequence SEQ ID AAsequence NO Aseune NO Aseune NO AseuneNO AAsqec 6 ANAAKNY 7 FNGIKEM 8 NNHTREV 9 TNKIRDP

TABLE 3
N2KR5 motif containing AAV9 7-mer variants enriched through in vivo transduction
screens in C57BL/6J and BALB/cJ mice using CMV-AAV-Express.

| SEQ | | SEQ | | SEQ | | SEQ | |
|---|---|---|---|---|---|---|---|
| ID NO | AA sequence | ID NO | AA sequence | ID NO | AA sequence | ID NO | AA sequence |
| 183 | ANAVRAI | 184 | GNSIRDT | 185 | LNSSRIP | 186 | SNAIKST |
| 187 | ANDIRPW | 188 | GNSIRTI | 189 | LNSVRHV | 190 | SNATRQV |
| 191 | ANHIRSL | 192 | GNSLRAY | 193 | LNTIRST | 194 | SNATRSI |
| 195 | ANTGRHN | 196 | GNSMRHM | 197 | LNTIRTN | 198 | SNEIRLV |
| 199 | ANYARND | 200 | GNSSRNL | 201 | LNTTRPI | 202 | SNFIRSA |
| 203 | DNAVRPW | 204 | GNSTRDH | 205 | LNTTRQV | 206 | SNHIRLA |
| 207 | DNEIRRW | 208 | GNSVKPS | 209 | LNTVRDI | 210 | SNHIRTL |
| 211 | DNHIRSQ | 212 | GNSVKQF | 213 | LNTVREP | 214 | SNHVRAI |
| 215 | DNHVKSL | 216 | GNSVRDF | 217 | LNTVRHI | 218 | SNHVRFM |
| 219 | DNKVKPT | 220 | GNSVRGW | 221 | LNVIRDP | 222 | SNHVRTS |
| 223 | DNNTRSV | 224 | GNSVRPV | 225 | LNYTRSS | 226 | SNNGRYM |
| 227 | DNRTKAL | 228 | GNTIRDI | 229 | MNDVRSV | 230 | SNNVRHY |
| 231 | DNRVRSP | 232 | GNTTKST | 233 | MNHIRTT | 234 | SNQTRSV |
| 235 | DNSTRAT | 236 | GNTVKDP | 237 | MNNARPY | 238 | SNSTKIL |
| 239 | DNSTRWS | 240 | GNTVKLL | 241 | MNQARNP | 242 | SNSTRSV |
| 243 | DNSVRLT | 244 | GNTVRDT | 245 | MNTIRSY | 246 | SNSVRAM |
| 247 | DNTVRGS | 248 | GNVIKTW | 249 | MNTVRDY | 250 | SNSVRDR |
| 251 | DNTWKAA | 252 | GNVIRSH | 253 | NNALKPY | 254 | SNTIKNL |
| 255 | ENATRSL | 256 | GNVTRST | 257 | NNASKGW | 258 | SNTIRTI |
| 259 | ENHLRNT | 260 | GNYGRAQ | 261 | NNATRNG | 262 | SNTTKAG |
| 263 | ENHVRNM | 264 | HNMSRIA | 265 | NNFTRGT | 266 | TNAVRGT |
| 267 | ENNTKIT | 268 | INDARTV | 269 | NNNGRSL | 270 | TNELRSY |
| 271 | ENPIRGR | 272 | INESRNR | 273 | NNSARPL | 274 | TNEVRLS |
| 275 | ENRIRDN | 276 | INHLKSA | 277 | NNSTRAG | 278 | TNFTKMT |
| 279 | ENSIKPL | 280 | INNERSK | 281 | NNSTRGG | 282 | TNGTKPT |
| 283 | ENSVRSA | 284 | INNVKSA | 285 | NNSTRGL | 286 | TNGTRNL |
| 287 | ENVVRSK | 288 | INNVRSV | 289 | NNTIRYS | 290 | TNHIRFT |
| 291 | GNATKAT | 292 | INPRKDN | 293 | NNTTRGM | 294 | TNHIRHL |
| 295 | GNAVKST | 296 | INQIRAA | 297 | NNTVRGM | 298 | TNHIRNL |
| 299 | GNAVRTG | 300 | INQIRQV | 301 | NNVIRGF | 302 | TNHSRPV |
| 303 | GNAVRTV | 304 | INSIRDL | 305 | PNDIRLR | 306 | TNMIRNA |
| 307 | GNDIRVR | 308 | INSIRMT | 309 | PNGIKGV | 310 | TNMPRNS |
| 311 | GNDIRYY | 312 | INTVRDR | 313 | PNNLRTP | 314 | TNPSRFA |
| 315 | GNDTRST | 316 | INYQKPA | 317 | PNNVRQY | 318 | TNSIRDQ |
| 319 | GNESRPT | 320 | INYVKHH | 321 | PNSIRRD | 322 | TNSTKAG |
| 323 | GNESRYM | 324 | KNLLRSE | 325 | PNSMRQV | 326 | TNSTRFT |
| 327 | GNEVRYY | 328 | LNDLRSR | 329 | PNSTRNV | 330 | TNSTRGV |
| 331 | GNFTRDL | 332 | LNEIRAV | 333 | PNTIRNV | 334 | TNSVRPV |
| 335 | GNGTRVY | 336 | LNEPRRV | 337 | PNTTRYL | 338 | TNTVRGG |
| 339 | GNHVRDA | 340 | LNEVKLY | 341 | QNDVRYP | 342 | TNVVKQT |
| 343 | GNLGRSS | 344 | LNHMRNT | 345 | QNEIKTY | 346 | TNYTKTL |
| 347 | GNLTKGY | 348 | LNNIRNV | 349 | QNKHREM | 350 | VNATRGG |
| 351 | GNMIRND | 352 | LNNIRQV | 353 | QNNIKAW | 354 | VNATRST |
| 355 | GNNTKAV | 356 | LNNVRPT | 357 | QNRMRND | 358 | VNEVRFQ |
| 359 | GNNTRSA | 360 | LNNVRSP | 361 | QNRQRDT | 362 | VNHDRAR |
| 363 | GNNVKGL | 364 | LNNVRSV | 365 | RNDGRVA | 366 | VNHIRLQ |
| 367 | GNNVKQF | 368 | LNQYRAA | 369 | RNDTRHI | 370 | VNHLREV |
| 371 | GNQTKPF | 372 | LNRVRGD | 373 | RNERRDV | 374 | VNHVRLT |
| 375 | GNRVKET | 376 | LNSARSI | 377 | RNEVRFA | 378 | VNNERSK |
| 379 | GNSARNI | 380 | LNSGRSA | 381 | RNTVKDP | 382 | VNTIRNV |
| 383 | GNSIRAL | 384 | LNSPRNV | 385 | SNAIKAT | 386 | VNTIRSV |
| | | | | | | 387 | YNQMRNT |

TABLE 4
N2KR5 motif containing AAV9 7-mer variants enriched through in vivo transduction
screens in marmosets using hSyn-AAV-Express.

| SEQ | | SEQ | | SEQ | | SEQ | |
|---|---|---|---|---|---|---|---|
| ID NO | AA sequence | ID NO | AA sequence | ID NO | AA sequence | ID NO | AA sequence |
| 388 | ANAERQP | 389 | GNETKSV | 390 | NNMVRTP | 391 | VNTVREF |
| 392 | ANDMRSG | 393 | GNETRML | 394 | NNSIKVG | 395 | VNTVREF |
| 396 | ANDSRAT | 397 | GNGTRLN | 398 | NNSLKEK | 399 | WNANRIN |
| 400 | ANGTKII | 401 | GNHVKQD | 402 | NNTVKNW | 403 | WNENKSM |
| 404 | ANITKSI | 405 | GNISKNI | 406 | NNVSRDS | 407 | YNAAKGV |
| 408 | ANLSKTT | 409 | GNLVKEN | 410 | NNVSRNN | 411 | YNDDRVF |
| 412 | DNAHRTQ | 413 | GNNIRNY | 414 | PNASRDW | 415 | YNERKEV |
| 416 | DNDIRGK | 417 | GNSARAV | 418 | PNNNKNH | 419 | YNESRNL |
| 420 | DNESKKS | 421 | GNVVKHM | 422 | PNTSKFT | 423 | YNEVKAN |
| 424 | DNKTRLT | 425 | GNYVRDH | 426 | QNALRST | 427 | YNHQKHD |
| 428 | DNNGRLT | 429 | HNLDRSN | 430 | QNALRST | 431 | YNLSRDD |
| 432 | DNNVRNV | 433 | HNLDRSN | 434 | QNGDKLP | 435 | YNMGKDH |
| 436 | DNPSRGI | 437 | HNLERQT | 438 | QNNIKDK | 439 | YNSIRNN |
| 440 | DNPVRGI | 441 | HNLERQT | 442 | QNVARNS | 443 | YNSNKPN |
| 444 | DNRHKEG | 445 | HNLHREN | 446 | RNADKGI | 447 | YNTQKYG |
| 448 | DNRIRGD | 449 | INETRNL | 450 | RNDARNS | | |
| 451 | DNSARET | 452 | INGDRAR | 453 | RNGSKSD | | |
| 454 | ENASKAF | 455 | INGSRNL | 456 | RNNVKAD | | |
| 457 | ENAVRSG | 458 | INKEKTI | 459 | RNTEKEQ | | |
| 460 | ENHLRNT | 461 | INMSKSA | 462 | SNDGRSN | | |
| 463 | ENHTRDK | 464 | INMSKSA | 465 | SNDQKDN | | |
| 466 | ENHVKQN | 467 | INSDRGD | 468 | SNEARSL | | |
| 469 | ENITKNV | 470 | KNTSRED | 471 | SNEPRVL | | |
| 472 | ENLERMV | 473 | LNATRNL | 474 | SNGDRSR | | |
| 475 | ENLIKQH | 476 | LNDIRNT | 477 | SNGGRLQ | | |
| 478 | ENLIRSN | 479 | LNDTRYY | 480 | SNGHKLS | | |
| 481 | ENLIRSN | 482 | LNGARIV | 483 | SNMNRIT | | |
| 484 | ENLLRSS | 485 | LNGARTD | 486 | SNMPRDT | | |
| 487 | ENLMRSS | 488 | LNGLRAH | 489 | SNNIRPI | | |
| 490 | ENLVKDD | 491 | LNGMKGA | 492 | SNNMRPA | | |
| 493 | ENNPRAT | 494 | LNGNRYS | 495 | SNNTKNF | | |
| 496 | ENNTKNY | 497 | LNGSRGA | 498 | SNPDRMK | | |
| 499 | ENQHRTF | 500 | LNGTRSL | 501 | SNTIKNL | | |
| 502 | ENTIRNT | 503 | LNLIKNN | 504 | TNAVKFT | | |
| 505 | ENTIRNT | 506 | LNMERTT | 507 | TNAVKTQ | | |
| 508 | ENTMKAH | 509 | LNNIKNT | 510 | TNDHKQY | | |
| 511 | ENYIRDK | 512 | LNSERSY | 513 | TNGGKYL | | |
| 514 | ENYIRDK | 515 | LNSLRGE | 516 | TNGIRNM | | |
| 517 | ENYVRER | 518 | LNVDRLI | 519 | TNGIRPW | | |
| 520 | FNDEKHT | 521 | MNGGKSL | 522 | TNGMRNQ | | |
| 523 | FNDGRTN | 524 | MNGTKAL | 525 | TNGVRNS | | |
| 526 | FNDNKAF | 527 | MNHSKSA | 528 | TNKERFN | | |
| 529 | FNGNKMH | 530 | MNMDRNT | 531 | TNMVKDW | | |
| 532 | FNGTRNT | 533 | MNSIRNT | 534 | TNQTKSI | | |
| 535 | FNNLKID | 536 | MNSLRSD | 537 | TNVVKAG | | |
| 538 | FNQMKNV | 539 | MNSTKIW | 540 | TNYTKDP | | |
| 541 | FNSDKSR | 542 | NNAPKSS | 543 | TNYTKDP | | |
| 544 | FNTHREG | 545 | NNGMRSS | 546 | VNAIRSQ | | |
| 547 | FNVTRVQ | 548 | NNHMRHD | 549 | VNEVRNY | | |
| 550 | GNATKDQ | 551 | NNLTRSL | 552 | VNSNKHD | | |

TABLE 5
N2KR5 motif containing AAV9 7-mer variants enriched through in vitro transduction
screening on hBMVECs using CMV-AAV-Express.

| SEQ | | SEQ | | SEQ | | SEQ | |
|---|---|---|---|---|---|---|---|
| ID NO | AA sequence | ID NO | AA sequence | ID NO | AA sequence | ID NO | AA sequence |
| 553 | ANHIRSL | 554 | INHIRML | 555 | LNYTRSS | 556 | SNLRRTE |
| 557 | ANLARVT | 558 | INHLKSA | 559 | MNEVRYA | 560 | SNMPRNN |
| 561 | ANTGRHN | 562 | INHMRGA | 563 | MNHIRTT | 564 | SNNGRYM |
| 565 | ANYKRET | 566 | INMKRVP | 567 | MNNARPY | 568 | SNNVRHY |
| 569 | DNARRTL | 570 | INPRKDN | 571 | MNQIRAA | 572 | SNPTRQY |
| 573 | DNAVRPW | 574 | INQIRAA | 575 | MNQSRGW | 576 | SNRQREL |
| 577 | DNAVRSK | 578 | INQIRQV | 579 | MNRSRAE | 580 | SNSHKGW |
| 581 | DNEIRRW | 582 | INSIRMT | 583 | MNRTRFE | 584 | SNTGKSW |
| 585 | DNERRPR | 586 | INSLRLV | 587 | MNSPRSY | 588 | SNTIKNL |
| 589 | DNHIRSQ | 590 | INSPRLF | 591 | MNTIRSY | 592 | SNTVRTI |
| 593 | DNNPRKS | 594 | INSPRVT | 595 | MNTVRAW | 596 | TNDGRSR |
| 597 | DNPNKRQ | 598 | INSQRTP | 599 | MNTVRDY | 600 | TNFGRTT |
| 601 | DNVRRSN | 602 | INSTRYP | 603 | MNVLKKG | 604 | TNHIRFT |
| 605 | ENHLRNT | 606 | INSYKGA | 607 | NNALKPY | 608 | TNHIRNL |
| 609 | ENRSKNN | 610 | INTSRSA | 611 | NNFSRNG | 612 | TNMIRNA |
| 613 | ENSIRMY | 614 | INTVRDR | 615 | NNLNKYL | 616 | TNMPRNS |
| 617 | ENVVRSK | 618 | INTVRSS | 619 | NNMPRGG | 620 | TNPARYG |
| 621 | FNGGRMG | 622 | KNAIKLG | 623 | NNNVRFL | 624 | TNPNRSS |
| 625 | FNKTRGP | 626 | KNLLRSE | 627 | NNPLKRV | 628 | TNPSRFA |
| 629 | FNRERNN | 630 | LNERKYT | 631 | NNSARPL | 632 | TNQLRGY |
| 633 | FNTKRDF | 634 | LNERRYQ | 635 | NNSTRAG | 636 | TNQLRRD |
| 637 | FNTLKLG | 638 | LNGGRSW | 639 | NNSTRGG | 640 | TNSGKWS |
| 641 | FNTPRTA | 642 | LNHIRLS | 643 | NNTIRYS | 644 | TNSTRFT |
| 645 | GNARRET | 646 | LNHIRSL | 647 | NNVIRGF | 648 | VNDLRTR |
| 649 | GNAVKST | 650 | LNHMRNT | 651 | NNYPRNM | 652 | VNGHRSN |
| 653 | GNAWKNA | 654 | LNLNRYS | 655 | PNMPRYE | 656 | VNHVRLT |
| 657 | GNDIRVR | 658 | LNMSKYA | 659 | PNNLRNV | 660 | VNMLRDP |
| 661 | GNESRYM | 662 | LNNIRNV | 663 | PNNVRQY | 664 | VNMPRSV |
| 665 | GNEVRYY | 666 | LNNIRQV | 667 | PNRYKDV | 668 | VNNMRYP |
| 669 | GNGVKWI | 670 | LNNLRSP | 671 | PNSIRRD | 672 | VNSPRTP |
| 673 | GNKFKQA | 674 | LNNTKTA | 675 | PNSYKNI | 676 | VNTIRNV |
| 677 | GNLGRSS | 678 | LNNVRMI | 679 | PNTIRNV | 680 | WNAPRNA |
| 681 | GNMIRND | 682 | LNNVRSP | 683 | PNTLRYA | 684 | WNENKRL |
| 685 | GNMPRSN | 686 | LNNVRSV | 687 | QNNIKAW | 688 | WNSPRTN |
| 689 | GNNTKAV | 690 | LNPRKIG | 691 | QNNNRPA | 692 | YNNFKGN |
| 693 | GNNVKQF | 694 | LNQYRAA | 695 | QNRMRND | 696 | YNRNKFE |
| 697 | GNSARNI | 698 | LNRERSA | 699 | QNRQRDT | 700 | YNSGRNT |
| 701 | GNSIRAL | 702 | LNRVRDH | 703 | QNSLRYS | 704 | YNTGRLV |
| 705 | GNSIRDT | 706 | LNRVRGD | 707 | RNDTRHI | | |
| 708 | GNSIRTI | 709 | LNSARSI | 710 | RNFERAN | | |
| 711 | GNSSRNL | 712 | LNSGRSA | 713 | RNGFREL | | |
| 714 | GNSVRGW | 715 | LNSLRHP | 716 | RNPAKTG | | |
| 717 | GNTIRDI | 718 | LNSVRHV | 719 | RNQDRTT | | |
| 720 | GNVIRSH | 721 | LNTFKAA | 722 | RNVDRST | | |
| 723 | GNVPRVF | 724 | LNTIRST | 725 | RNYIKSD | | |
| 726 | GNVTRST | 727 | LNTLRTI | 728 | SNDGRYY | | |
| 729 | GNYGRAQ | 730 | LNTSRSW | 731 | SNHIRLA | | |
| 732 | GNYSRMD | 733 | LNTVRHI | 734 | SNHIRTL | | |
| 735 | HNDTKRY | 736 | LNYNKGY | 737 | SNHVRAI | | |
| 738 | INAPRTA | 739 | LNYTRPA | 740 | SNHVRFM | | |

TABLE 6
N2KR5 motif containing AAV9 7-mer variants enriched through in vitro transduction
screening on mBMVECs using CMV-AAV-Express.

| SEQ ID NO | AA sequence | SEQ ID NO | AA sequence | SEQ ID NO | AA sequence |
|---|---|---|---|---|---|
| 741 | ANEVRRG | 742 | LNHIRSL | 743 | QNRMRND |
| 744 | ANHIRSL | 745 | LNHMRNT | 746 | QNTARFM |
| 747 | ANNNRNY | 748 | LNNIRNV | 749 | RNDTRHI |
| 750 | ANYKRET | 751 | LNNIRQV | 752 | RNYIKSD |
| 753 | CNGTKRE | 754 | LNNVRMI | 755 | SNHIRTL |
| 756 | CNRSRDG | 757 | LNNVRSV | 758 | SNHVRAI |
| 759 | DNARRTL | 760 | LNPRKIG | 761 | SNHVRTS |
| 762 | DNAVRSK | 763 | LNPVRNA | 764 | SNLRRTE |
| 765 | DNEIRRW | 766 | LNQYRAA | 767 | SNNVRHY |
| 768 | DNHIRSQ | 769 | LNRVRDH | 770 | SNPTRQY |
| 771 | ENHLRNT | 772 | LNRVRGD | 773 | SNRQREL |
| 774 | ENPPRPR | 775 | LNSARSI | 776 | SNSIRFI |
| 777 | ENRSKNN | 778 | LNSGRSA | 779 | SNTIKNL |
| 780 | ENSIRMY | 781 | LNSRREV | 782 | SNTIRTI |
| 783 | ENYGRGA | 784 | LNSVRHV | 785 | SNTVRTI |
| 786 | GNAVKST | 787 | LNTFKAA | 788 | TNFGRTT |
| 789 | GNDIRVR | 790 | LNTIRST | 791 | TNHIRFT |
| 792 | GNDIRYY | 793 | LNTIRTN | 794 | TNHIRNL |
| 795 | GNEVRYY | 796 | LNTIRVV | 797 | TNMIRNA |
| 798 | GNGVKWI | 799 | LNTVRDI | 800 | TNSTRFT |
| 801 | GNMIRND | 802 | LNTVRHI | 803 | TNVLRGF |
| 804 | GNNTKAV | 805 | LNYTRSS | 806 | VNHIRLQ |
| 807 | GNNVKQF | 808 | MNDVRSV | 809 | VNHVRLT |
| 810 | GNSARNI | 811 | MNHIRTT | 812 | VNNERSK |
| 813 | GNSIRAL | 814 | MNHPRMI | 815 | VNSPRSI |
| 816 | GNSIRDT | 817 | MNNARPY | 818 | VNTIRNV |
| 819 | GNSIRTI | 820 | MNQSRGW | 821 | VNTIRSV |
| 822 | GNSMRHM | 823 | MNRTRFE | 824 | WNSPRTN |
| 825 | GNSSRNL | 826 | MNSPRSL | 827 | YNRNKFE |
| 828 | GNSVRGW | 829 | MNTIRSY | 830 | YNSGRNT |
| 831 | GNTIRDI | 832 | MNTVRAW | 833 | YNTGRLV |
| 834 | GNVIRSH | 835 | MNTVRDY | | |
| 836 | GNVMRTT | 837 | NNASKGW | | |
| 838 | GNVTRST | 839 | NNFSRNG | | |
| 840 | GNYGRAQ | 841 | NNNVRFL | | |
| 842 | HNMSRIA | 843 | NNPVRIP | | |
| 844 | INHLKSA | 845 | NNSARPL | | |
| 846 | INHMRGA | 847 | NNSTRAG | | |
| 848 | INMPRDT | 849 | NNSTRGG | | |
| 850 | INNARIP | 851 | NNTIRYS | | |
| 852 | INQIRQV | 853 | NNVIRGF | | |
| 854 | INSPRLF | 855 | PNHVRIA | | |
| 856 | INSPRVS | 857 | PNNLRNV | | |
| 858 | INSPRVT | 859 | PNNVRQY | | |
| 860 | INTSRSA | 861 | PNSIRFS | | |
| 862 | INTVRDR | 863 | PNSIRRD | | |
| 864 | INTVRSS | 865 | PNTIRNV | | |
| 866 | KNAIKLG | 867 | PNTLRYA | | |
| 868 | KNNLREY | 869 | QNLGRYV | | |
| 870 | LNEVKLY | 871 | QNNIKAW | | |

TABLE 7
N2KR5 motif containing AAV9 7-mer variants enriched through in vitro transduction
screening on human CMEC/D3 cells using CMV-AAV-Express.

| SEQ | | SEQ | | SEQ | | SEQ | |
|---|---|---|---|---|---|---|---|
| ID NO | AA sequence | ID NO | AA sequence | ID NO | AA sequence | ID NO | AA sequence |
| 872 | ANGTRGH | 873 | INHVRCV | 874 | LNYNKGY | 875 | SNHIRLA |
| 876 | ANHIRSL | 877 | INIIRVP | 878 | LNYTRPA | 879 | SNHIRTL |
| 880 | ANKNKPV | 881 | INITRNH | 882 | LNYTRSS | 883 | SNHRRME |
| 884 | ANRDRYT | 885 | INMKRVP | 886 | MNHIRTT | 887 | SNHVRAI |
| 888 | ANSQRHS | 889 | INPKRTA | 890 | MINHPRMI | 891 | SNHVRFM |
| 892 | ANYKRET | 893 | INQIRAA | 894 | MNHYRPQ | 895 | SNHVRTS |
| 896 | CNKPKDN | 897 | INSIRMT | 898 | MNLSKYP | 899 | SNKNRWE |
| 900 | DNARRVT | 901 | INSKKNA | 902 | MNMPRTS | 903 | SNLRRTE |
| 904 | DNAVRSK | 905 | INSPRVT | 906 | MNNARPY | 907 | SNNGRYM |
| 908 | DNEIRRW | 909 | INSTRYP | 910 | MINNSRMP | 911 | SNNVRHY |
| 912 | DNERRPR | 913 | INSVRIP | 914 | MNQSRGW | 915 | SNRDRNS |
| 916 | DNHIRSQ | 917 | INSYKGA | 918 | MNRSRAE | 919 | SNRNRDY |
| 920 | DNNPRKS | 921 | INTVRSS | 922 | MNRTRFE | 923 | SNSHKGW |
| 924 | DNVRRSN | 925 | INYQKPA | 926 | MNSPRSL | 927 | SNSIRFI |
| 928 | ENDKKNK | 929 | INYVKHH | 930 | MNSPRSY | 931 | SNTGKSW |
| 932 | ENMPRPT | 933 | KNAIKLG | 934 | MNTIRSY | 935 | SNTIKNL |
| 936 | ENNGRRN | 937 | KNALKSS | 938 | MNTVRAW | 939 | SNTIRTI |
| 940 | ENNRRQM | 941 | KNDERHR | 942 | MNVLKKG | 943 | TNERKYL |
| 944 | ENRQKYT | 945 | KNGGKPN | 946 | NNFSRNG | 947 | TNFGRTT |
| 948 | ENRSKNN | 949 | KNHNKPG | 950 | NNILRVG | 951 | TNHIRFT |
| 952 | ENSLRHR | 953 | KNLMRID | 954 | NNMPRGG | 955 | TNHIRHL |
| 956 | FNGGRMG | 957 | KNPIKSS | 958 | NNPLKRV | 959 | TNHIRHP |
| 960 | FNKTRGP | 961 | LNDSRRP | 962 | NNPVRIP | 963 | TNHIRNL |
| 964 | FNRERGP | 965 | LNERRYQ | 966 | NNSTRAG | 967 | TNKTRPA |
| 968 | FNRERNN | 969 | LNHARYP | 970 | NNSTRGG | 971 | TNMPRNS |
| 972 | FNTKRDF | 973 | LNHGRTA | 974 | NNTIRYS | 975 | TNPARYG |
| 976 | FNTPRTA | 977 | LNHIRLS | 978 | NNTYRSS | 979 | TNQLRRD |
| 980 | GNAVKST | 981 | LNHIRSL | 982 | NNVIRGF | 983 | TNSGKWS |
| 984 | GNAWKNA | 985 | LNHIRTS | 986 | PNEYKAR | 987 | TNSIRLP |
| 988 | GNDIRVR | 989 | LNHMRNT | 990 | PNHPRHL | 991 | TNSLRHP |
| 992 | GNKFKQA | 993 | LNIRRGE | 994 | PNMPRYE | 995 | TNSLRSI |
| 996 | GNLGRSS | 997 | LNKLRGP | 998 | PNNLRTP | 999 | TNSTRFT |
| 1000 | GNLQRYQ | 1001 | LNLNRYS | 1002 | PNNVRQY | 1003 | VNEVRMA |
| 1004 | GNMIRND | 1005 | LNMPRTN | 1006 | PNRYKDV | 1007 | VNHIRLQ |
| 1008 | GNNCKAT | 1009 | LNMSKYA | 1010 | PNSLRAI | 1011 | VNHVRLT |
| 1012 | GNNTKAV | 1013 | LNNIRNV | 1014 | PNSLRER | 1015 | VNLHRSG |
| 1016 | GNNVKQF | 1017 | LNNIRQV | 1018 | PNSMRQV | 1019 | VNNLRTL |
| 1020 | GNSARNI | 1021 | LNNVRSP | 1022 | PNTIRNV | 1023 | VNNMRYP |
| 1024 | GNSIRAL | 1025 | LNNVRSV | 1026 | PNTLRFA | 1027 | VNPNRSG |
| 1028 | GNSIRDT | 1029 | LNPRKIG | 1030 | QNKHREM | 1031 | VNSLRQY |
| 1032 | GNSIRTI | 1033 | LNPVRNA | 1034 | QNNIKAW | 1035 | VNTGKGW |
| 1036 | GNSLRAY | 1037 | LNQYRAA | 1038 | QNNLKYL | 1039 | VNTIRNV |
| 1040 | GNSSRNL | 1041 | LNRERSA | 1042 | QNRMRND | 1043 | VNTPRHS |
| 1044 | GNSVRGW | 1045 | LNRVRDH | 1046 | QNRQRDT | 1047 | WNAPRNA |
| 1048 | GNTIRDI | 1049 | LNRVRGD | 1050 | QNSLRYS | 1051 | WNEYRSS |
| 1052 | GNTYRDY | 1053 | LNSARSI | 1054 | QNTARFM | 1055 | WNHPRAA |
| 1056 | GNVIRSH | 1057 | LNSLRHP | 1058 | RNDGRVA | 1059 | WNNFRPS |
| 1060 | GNVPRVF | 1061 | LNSPRDR | 1062 | RNEVRFA | 1063 | WNPGRAG |
| 1064 | GNVTRST | 1065 | LNSPRFV | 1066 | RNEVRFG | 1067 | WNSNRFE |
| 1068 | GNYGRAQ | 1069 | LNSPRIT | 1070 | RNFNKND | 1071 | WNSPRNT |
| 1072 | HNDTKRY | 1073 | LNSRREV | 1074 | RNMAKAP | 1075 | WNSPRTN |
| 1076 | HNEERKR | 1077 | LNSVRHV | 1078 | RNNGRPQ | 1079 | YNAHRGA |
| 1080 | INAPRTA | 1081 | LNTFKAA | 1082 | RNNPKPL | 1083 | YNMPRGA |
| 1084 | INDLRTP | 1085 | LNTIRST | 1086 | RNPAKTG | 1087 | YNNFKGN |
| 1088 | INHIRML | 1089 | LNTPRST | 1090 | RNVDRST | 1091 | YNQMRNT |
| 1092 | INHLKSA | 1093 | LNTSRSW | 1094 | RNYIKSD | 1095 | YNRNKFE |
| 1096 | INHMRGA | 1097 | LNTVRHI | 1098 | SNFIRSA | 1099 | YNTGRLV |

### AAV-B130 Efficiently Transduces Brain Endothelial Cells in Mice and Rats In Vivo.

To develop capsids with improved transduction of CNS endothelial cells, Applicants generated an AAV9 capsid library and selected for capsids that more efficiently transduced human and mouse endothelial cells. The library comprised AAV9 variants modified with a randomized 7-mer insertion between amino acids 588 and 589 (AAV9 VP1 position). The library was built within a recombinant AAV backbone (AAV9-CMV-Express, see Methods for additional details) that expresses the capsid gene in transduced cells. By sequencing capsid mRNA this approach allows for the selective recovery of functional capsids, eliminating AAV variants that traffic to the tissue or organ of interest but fail to achieve transgene expression. Similar RNA-based selection methods have recently been used to identify capsids with enhanced blood-brain barrier penetrance (Nonnenmacher, M. et al. (2020), Mol Ther Methods Clin Dev 20, 366-378) and muscle transduction (Tabebordbar, M. et al. 2021, Cell 184, 4919-4938.e22).

Using AAV9-CMV-Express, Applicants selected for capsids expressed in human and mouse cells in vitro and in the brains of mice in vivo. After two rounds of selection, Applicants identified a variant with the 7-mer amino acid sequence NNSTRGG (SEQ ID NO: 1) that was enriched in the expressed capsid pool across all assays: in hCMEC/D3 transduction, in both human and mouse brain microvascular endothelial cell (BMVEC) transduction, and in C57BL/6J and BALB/cJ mouse brain transduction in vivo (FIG. 3). In stark contrast, AAV-PHP.eB, a previously-described capsid (Chan, K. Y. et al. 2017), Nat Neurosci 20, 1172-1179) with enhanced CNS transduction selective to a subset of mice including C57BL/6J's (Huang, Q. et al. 2019, PLoS One 14, e0225206; Hordeaux, J. et al. 2019, Mol Ther 27, 912-921), was only enriched in C57BL/6J brain and BMVECs derived from this strain.

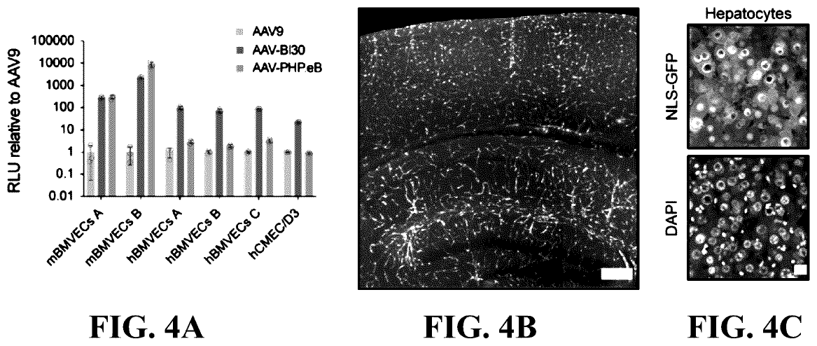

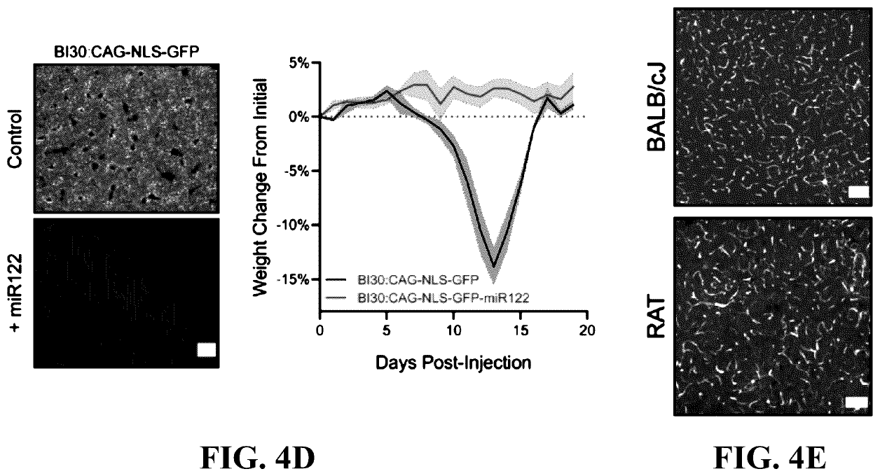

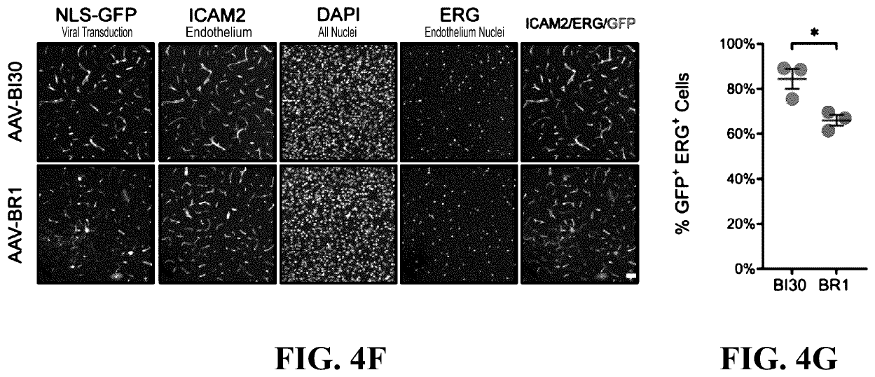

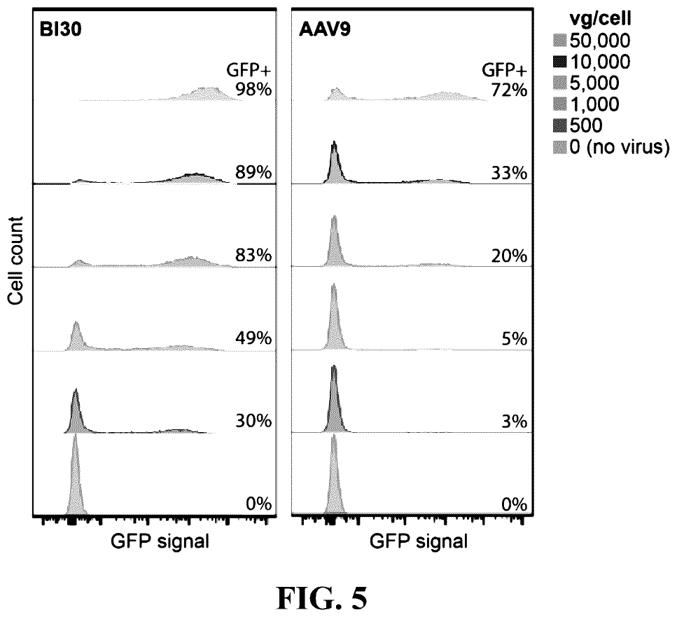

To individually assess the transduction characteristics of AAV-B1I30, Applicants used the capsid to package a single-stranded recombinant AAV2 reporter genome. AAV-B130 transduced multiple lots of BMVECs from mouse (282- to 2261-fold) and human (72- to 96-fold) more efficiently than AAV9 (FIG. 4A). In addition, AAV-B130 transduced immortalized human cerebral microvascular endothelial cells (hCMEC/D3) (22.7±1.4-fold; mean±SD) more efficiently than AAV9, an increase that was observed across a wide range of doses (FIG. 5). Fitting with our library enrichment data, this cross-species transduction enhancement differentiates AAV-BI30 from AAV-PHP.eB, which exhibited an enhanced transduction phenotype restricted to mouse BMVECs (FIG. 4A), and AAV-BR1, which was not found to transduce hCMEC/D3 cells more efficiently than its parental vector AAV2 (Körbelin, J. et al. 2016, EMBO Mol Med 8, 609-25).

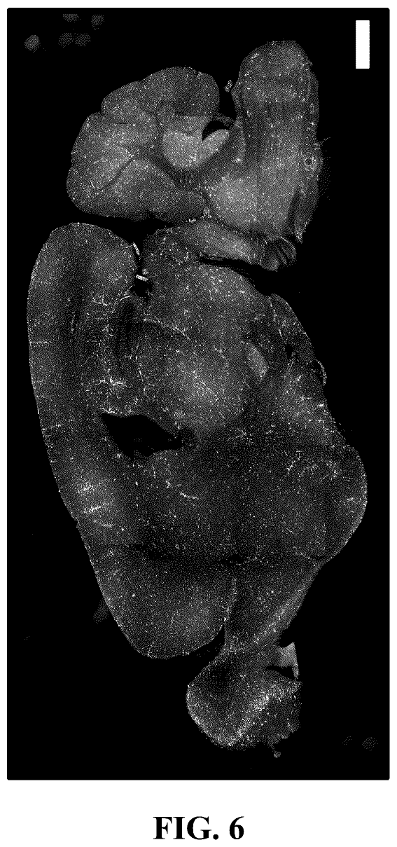

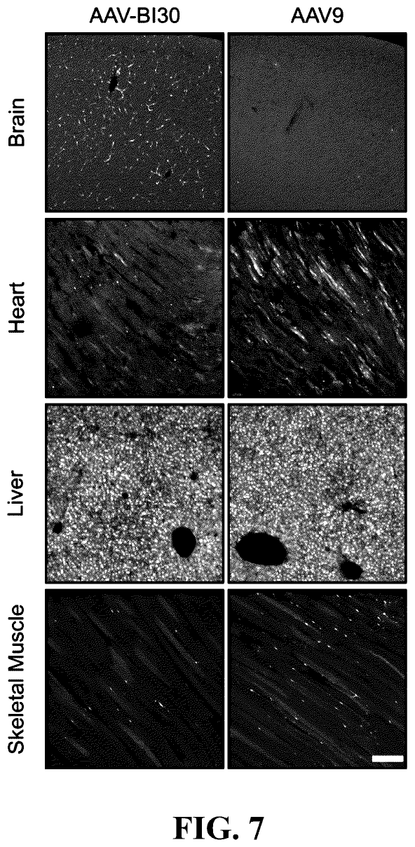

To evaluate AAV-B1I30's performance in vivo, Applicants then used the capsid to package a genome expressing a nuclear localization signal (NLS) tagged GFP from the ubiquitous CAG promoter (AAV-BI30:CAG-NLS-GFP). Applicants then intravenously administered the AAV at 1×10¹¹ vg/mouse in C57BL/6 mice and assessed transduction after 10 days. Encouragingly, AAV-B130 transduced endothelial cells throughout the brain with remarkable efficiency and specificity at this dose (FIG. 4B, FIG. 6 and FIG. 7).

Approximately one-week post-administration, Applicants however observed unexpected dose-dependent toxicity at doses as low as 1×10¹¹ vg/mouse. This adverse response manifested in weight loss, lethargic behavior, and ultimately mortality at the highest dose tested, 1×10¹²vg. Necropsy revealed strong transduction of liver hepatocytes accompanied by abnormal nuclear morphology (FIG. 4C). To determine whether toxic overexpression of the NLS-transgene in hepatocytes contributed to systemic toxicity, Applicants incorporated three repeats of the hepatocyte-specific miR-122 target sequence (Lagos-Quintana, M. et al. (2002), Curr Biol 12, 735-739; Landgraf, P. et al. (2007), Cell 129, 1401-1414) into the 3′UTR of our viral construct—a strategy successfully employed by a number of groups to selectively degrade transgene mRNA in hepatocytes (Suzuki, T. et al. 2008, Mol Ther 16, 1719-1726; Xie, J. et al. 2011, Mol Ther 19, 526-535; Geisler, A. et al. (2011), Gene Ther 18, 199-209).

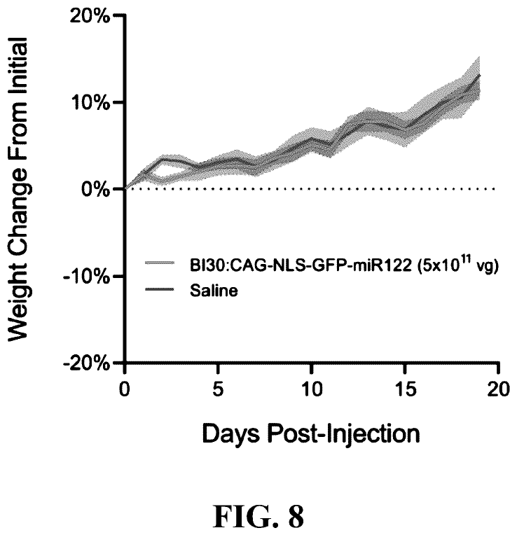

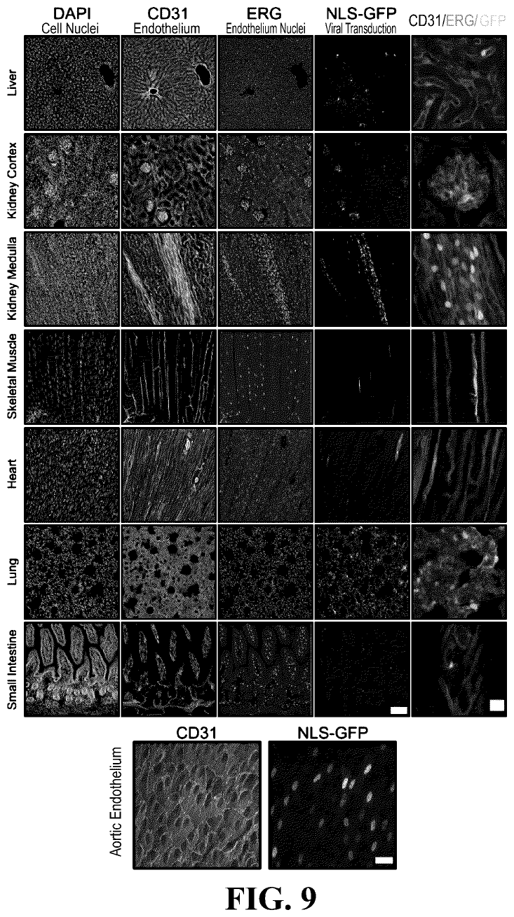

This modification efficiently suppressed hepatocyte expression of the NLS-GFP transgene and prevented transient weight loss (FIG. 4D) without compromising AAV-BI30's transduction of the CNS vasculature, results consistent with virtually undetectable expression of Mir122a in brain endothelial cells (Vanlandewijck, M. et al. 2018, Nature 554, 475-480; Zhang, Y. et al. 2014, JNeurosci 34, 11929-11947). A higher 5×10¹ vg dose of AAV-B130 carrying the modified NLS-GFP-miR122-WPRE construct similarly produced no discernable weight loss (FIG. 8), illustrating the microRNA binding element's ability to effectively detarget transgene expression from the liver across the experimental working range of the vector. Further, measuring less than 80 bp, the element did not appreciably constrain AAV-BI30's functional packaging capacity. A survey of peripheral tissues following incorporation of the miR-122 repeat element revealed transduction of several non-CNS endothelial populations in addition to hepatocytes, including endothelial cells in the lung microvasculature, aorta, and interlobular vessels of the kidney. That said, the vector's transduction profile was strongly biased towards the CNS (FIG. 9).

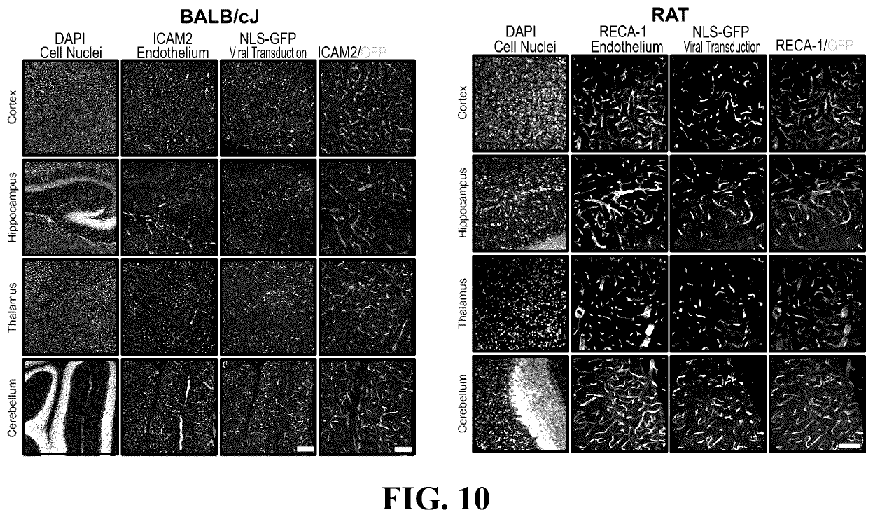

C57BL/6-restricted tropism has frustrated past efforts to deploy engineered AAV vectors in genetically-intractable organisms (Hordeaux, J. et al. 2018, Mol Ther 26, 664-668; Matsuzaki, Y. et al. 2018, Neurosci Lett 665, 182-188). To test whether AAV-BI30's potential applications were similarly constrained, Applicants evaluated the capsid's performance in a second mouse strain (BALB/cJ) and a distinct mammalian species (rat). Consistent with AAV-BI30's cross-species transduction observed in vitro, the capsid achieved robust endothelial transduction in the BALB/cJ and rat brain following systemic administration (FIG. 4E and FIG. 10).

### AAV-B130 Efficiently Transduces Brain Endothelial Cells Across the Entire Arterio-Venous Axis.

Applicants subsequently sought to assess AAV-BI30's transduction efficiency in brain endothelial cells. Previous studies have quantified the efficiency of endothelial-targeted vectors by measuring the co-localization of a viral transgene with endothelial-specific markers such as CD31 or GLUT1 (Kumar, S. R. et al. (2020), Nat Methods 17, 541-550; Körbelin, J. et al. (2016), EMBO Mol. Med. 8, 609-625). This strategy relies on an implicit assumption that the size and morphology of individual endothelial cells is roughly consistent throughout the CNS—a generalization that does not hold true across the arterio-venous axis (dela Paz, N. G. et al. (2008), Cell Tissue Res 335, 5-16). Seeking to improve upon existing approaches, Applicants developed an automated Cell Profiler-based (McQuin, C. et al. (2018), PLoS Biol 16, e2005970) workflow to estimate endothelial transduction efficiency by measuring co-localization of NLS-GFP with ERG, an endothelial-specific ETS family transcription factor (Vanlandewijck, M. et al. (2018), Nature 554, 475-480; Shah, A. V. et al. (2016), Vasc. Pharmacol 86, 3-13). Because ERG expression is sharply restricted to the nucleus, this strategy enabled fast and reliable identification of individual endothelial cells throughout the brain microvasculature (FIG. 4F).

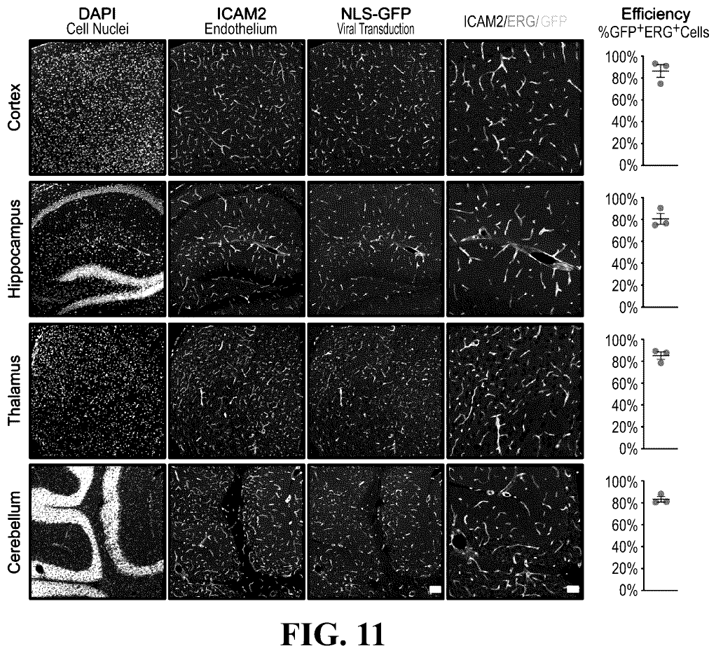

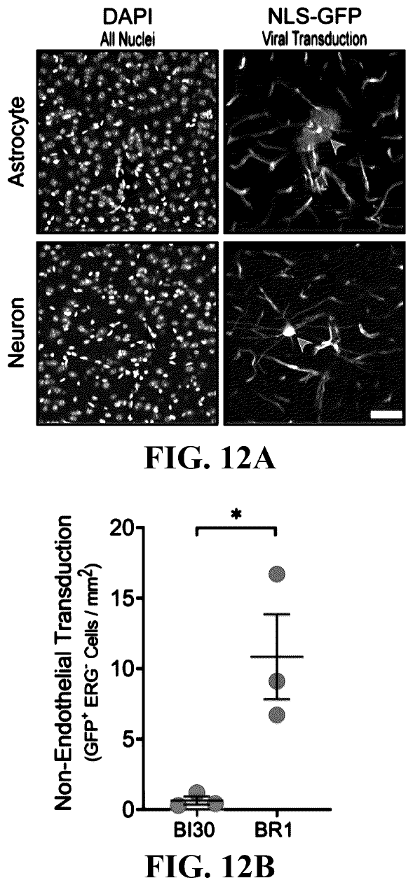

Observing that AAV-BI30's tropism resembled AAV-BR1's despite its highly divergent sequence, Applicants used this approach to directly compare the vectors and identify unique capsid-specific properties. Quantifying transduction across entire sagittal sections of brain, Applicants found that AAV-B130 transduced 84±4% (mean s.e.m.) of brain endothelial cells at 1×10¹¹ vg/mouse. By comparison, AAV-BR1 transduced 66±2% of this population at the same dose, consistent with previous reports (Körbelin, J. et al. (2016), EMBO Mol. Med. 8, 609-625; Tan, C. et al. (2019), Neuron 101, 920-937.e13; Santisteban, M. M. et al. (2020); Hypertension 76, 795-807; Nikolakopoulou, A. M. et al. (2021), J Exp Med 218, e20202207) (FIG. 4G). AAV-BI30's efficacy showed no appreciable region-to-region variation throughout the brain; cortex, hippocampus, thalamus, and cerebellum all exhibited>80% endothelial cell transduction (FIG. 11). Further, the vector was highly endothelial-specific in this dose regime—isolated instances of neuronal or astrocytic transduction were rare (FIG. 12). Notably, while both AAV-B130 and AAV-BR1 were predominantly endothelial-directed, AAV-B130 transduced significantly fewer non-endothelial cells than AAV-BR1 (which is known to sporadically transduce neurons and astrocytes (Körbelin, J. et al. (2016), EMBO Mol. Med. 8, 609-625; Graßhoff, H. et al. (2021), J Cereb Blood Flow Metab. doi:10.1177/0271678X211039617).

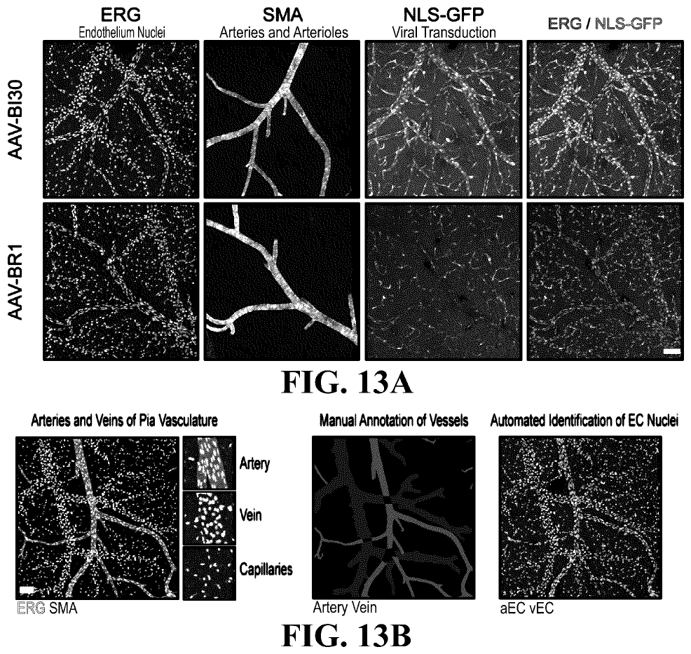

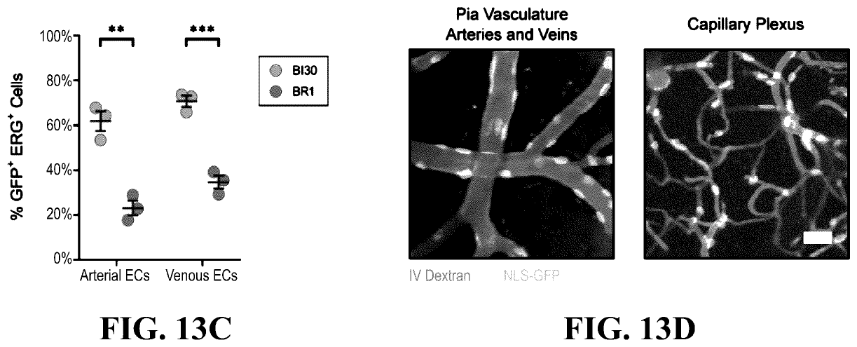

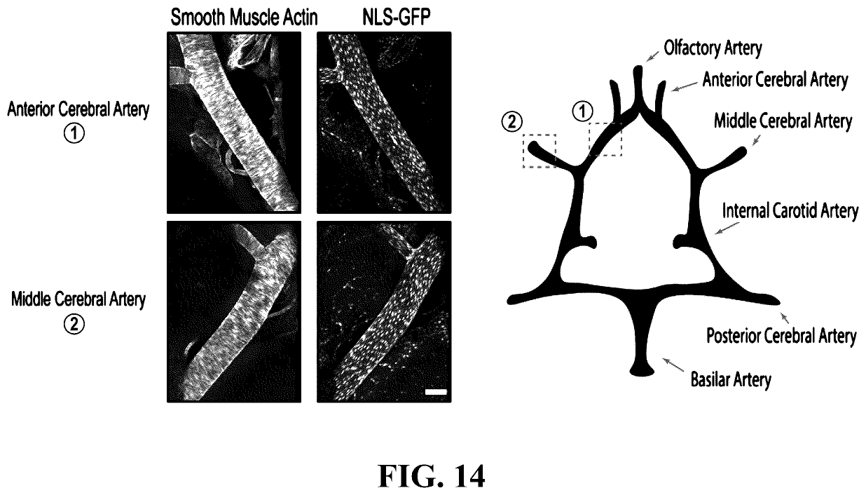

Applicants then measured AAV-B1I30's transduction efficiency in endothelial cells of larger arteries and veins. While surveying sagittal or coronal sections is well-suited to the gauge the overall efficiency of AAV transduction across the brain microvasculature, it provides limited information about a vector's ability to transduce different segments of the arterio-venous axis. Capillaries, the brain's smallest blood vessels, constitute the vast bulk of the cerebrovascular network. Furthermore, arteries and veins are disproportionately confined to the pia surface and poorly sampled by sectioning approaches. As a result, the overwhelming majority of microvessels surveyed in a given sagittal or coronal plane are capillaries. Therefore, to assess AAV-B130 and AAV-BR1's ability to target the endothelium of arteries and veins Applicants examined whole-mount preparations of the intact pia vasculature (FIG. 13A). Because ERG is ubiquitously expressed across arteries, capillaries, and veins our analysis workflow could be rapidly adapted to calculate segment-specific transduction efficiency. Applicants manually identified arteries and veins based on (i) the presence of α-Smooth Muscle Actin (high in arteries, low-to-absent in veins) (Vanlandewijck, M. et al. (2018), Nature 554, 475-480; Hill, R. A. et al. (2015), Neuron 87, 95-110) and (ii) the nuclear morphology of endothelial cells (ellipsoidal in arteries, circular in veins) (dela Paz, N. G. et al. (2008), Tissue Res 335, 5-16 (FIG. 13B). Strikingly, Applicants found that AAV-B130 captured these vessel segments efficiently, transducing 62±4% of arterial ECs and 71±3% of venous ECs. By contrast, AAV-BR1's tropism appeared strongly biased against large-vessel transduction: the vector only transduced 23±3% of arterial ECs and 35±3% of venous ECs in the pia vasculature (FIG. 13C). In line with these findings, Applicants were able to visualize robust AAV-B130-mediated endothelial GFP expression in the arteries, capillaries, and veins of live mice via two-photon imaging through surgically implanted cranial windows (FIG. 13D). Interestingly, AAV-B1I30's tropism extended to the largest arteries of the brain; at a higher 5×10¹¹ vg dose of the vector Applicants observed efficient endothelial transduction throughout the Circle of Willis and associated cerebral arteries (FIG. 14). Collectively, these results demonstrate that AAV-B130 can be leveraged to genetically interrogate the majority of brain endothelial cells across the entire arterio-venous axis at relatively low systemic doses.

### AAV-B130 Efficiently Transduces Endothelial Cells in Retina and Spinal Cord.

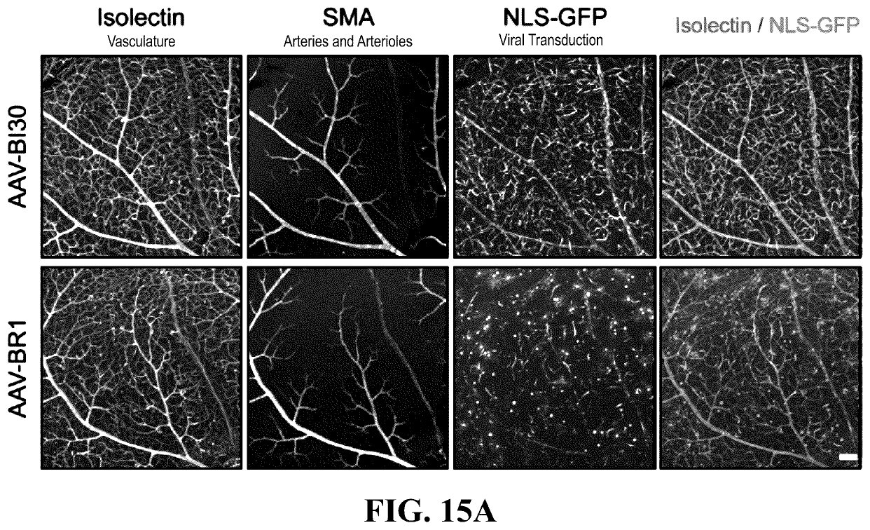

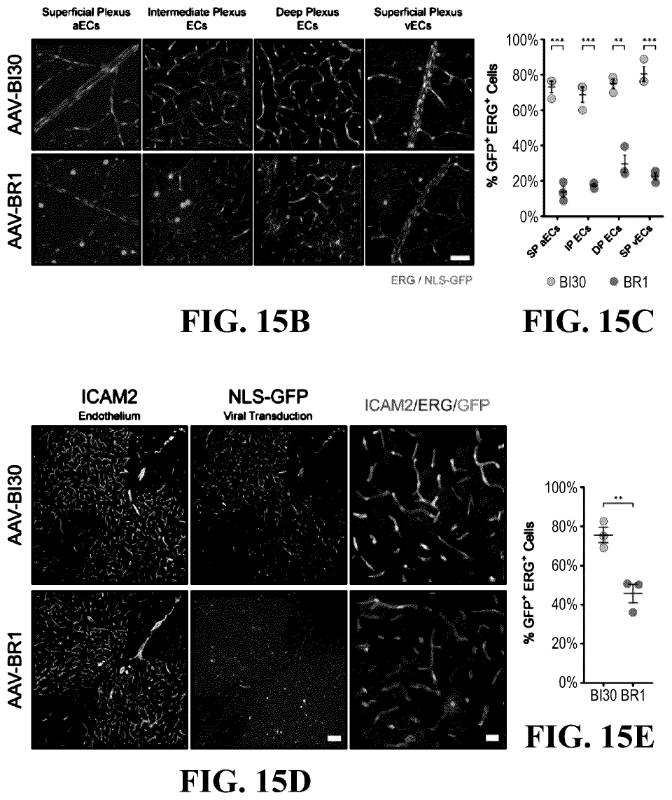

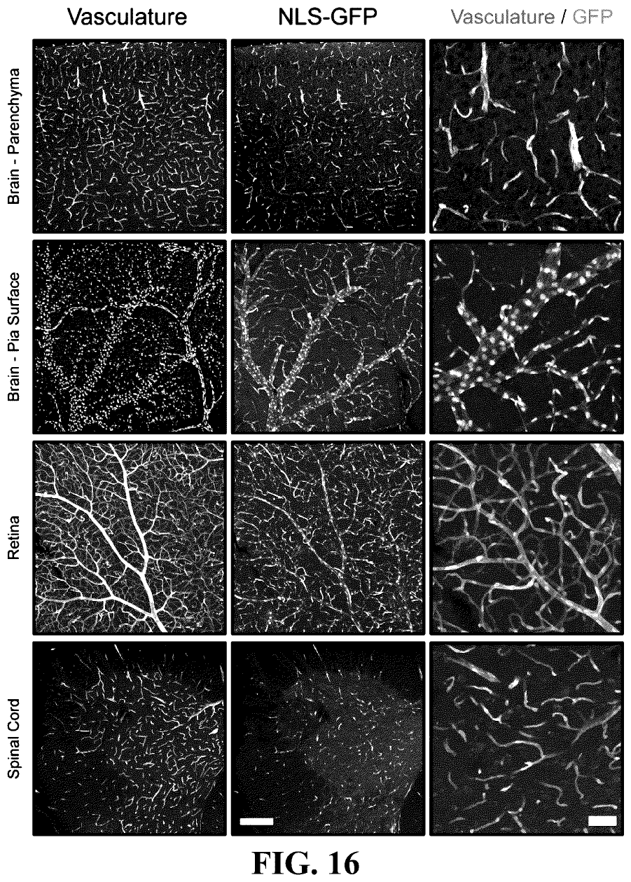

While the majority of endothelial-targeted AAV research to-date has focused on the brain vasculature, the retina and spinal cord vasculature are highly-tractable systems crucial to the study of angiogenesis, blood-brain barrier dynamics, neurovascular pathology, and a host of other key processes (Chow, B. et al. (2017), Neuron 93, 1325-1333.e3; Bartanusz, V., et al. (2011), Ann Neurol 70, 194-206; Stahl, A. et al. (2010), Invest Ophthalmol Vis Sci 51, 2813-2826; Newman, E. A. (2013), J Cereb Blood Flow Metab 33, 1685-1695). Accordingly, Applicants investigated AAV-B1I30's capacity to transduce the endothelial cells of these CNS tissues. Across all segments of the retina's stereotyped vasculature, AAV-B130 dramatically outperformed AAV-BR1, transducing 73±3% versus 14±3% superficial plexus arterial ECs; 69±4% versus 18±1% intermediate plexus ECs; 75±3% versus 30±5% deep plexus ECs; and 81±4% versus 23±2% superficial plexus venous ECs (FIG. 15A-C). The difference between the vectors was similarly apparent in the spinal cord, where AAV-B130 transduced 76±4% of ECs compared to AAV-BR1's 46±5%—an estimate consistent with the capsid's initial characterization² (FIG. 15D-E). Thus, AAV-B1I30's highly efficient, endothelial cell-specific tropism is not limited to the brain; instead, it extends the entirety of the CNS. In addition, AAV-B130-mediated transgene expression persists across long timescales. Applicants observed robust endothelial transduction in brain, retina, and spinal cord 152 days after administration of a single 1×10¹¹ vg dose of the vector (FIG. 16), a result consistent with the relatively slow turnover of CNS endothelial cells (Hobson, B. (1984), Br J Cancer 49, 405-413) and longitudinally-stable endothelial transduction previously demonstrated with AAV-BR1 (Körbelin, J. et al. (2016), EMBO Mol Med 8, 609-25).

### AAV-B130 can be Leveraged to Achieve Efficient Brain Endothelial-Specific Gene Manipulation.

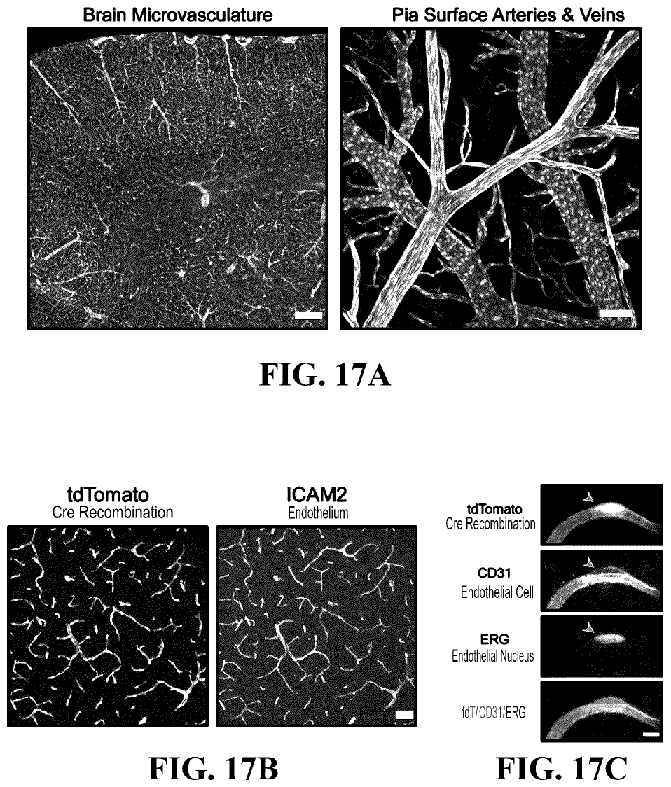

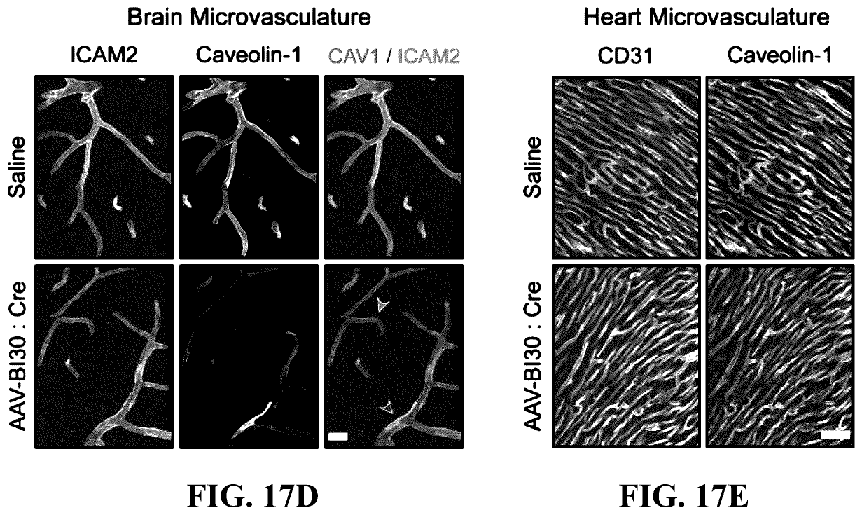

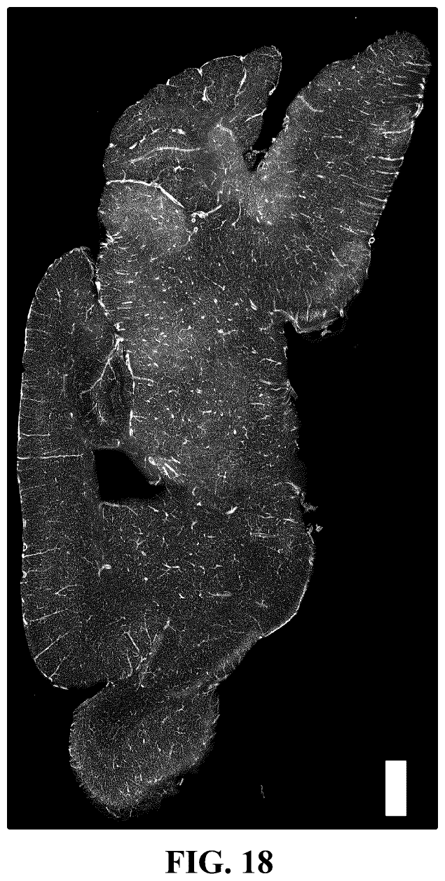

To evaluate AAV-B1I30's capacity to genetically manipulate CNS endothelial cells in vivo, Applicants used the capsid to package Cre recombinase (AAV-BI30:CAG-Cre-miR122-WPRE) and delivered a 1×10¹¹ vg dose of the vector to Rosa26:CAG-LSL-tdTomato (Ai9) Cre-dependent reporter mice (Madisen, L. et al. (2010), Nat Neurosci 13, 133-140). Consistent with our prior results, Applicants observed remarkably efficient, endothelial-specific tdTomato expression throughout the brain (FIG. 17A-B, FIG. 18). By quantifying ERG co-localization with tdTomato, Applicants found that AAV-B130-mediated Cre delivery drove recombination in 94±1% of endothelial cells within the cortical microvasculature (FIG. 17C).

Applicants next tested this strategy's ability to achieve acute genetic loss-of-function in CNS endothelial cells. For a proof-of-concept experiment Applicants chose to target Caveolin-1 (encoded by the Cav1 gene), an essential component of caveolae (Fra, A. M., (1995), Proc Natl Acad Sci USA 92, 8655-8659; Razani, B. et al. (2001), J Biol Chem 276, 38121-38138). Caveolae are flask-shaped vesicular invaginations of the plasma membrane found in a number of cell types, including endothelial cells. Within CNS endothelial cells, these subcellular structures play a key role in blood-brain barrier dynamics (Andreone, B. J. et al. (2017), Neuron 94, 581-594; Chow, B. et al. (2017), Neuron 93, 1325-1333.e3; Sadeghian, H. et al. (2018), Ann Neurol 84, 409-423) and neurovascular coupling (Chow, B. W. et al. (2020), Nature 579, 106-110) among other important functions.

To genetically ablate Caveolin-1 expression, Applicants delivered a 1×10¹¹ vg dose of AAV-B130 carrying a CAG-Cre-miR122-WPRE genome to Cav1^{fl/fl} mice (Asterholm, I. et al. (2012), Cell Metab 15, 171-185). Four weeks post-administration Applicants observed strong reduction of Caveolin-1 protein in the brain endothelial cells of AAV-B130-injected animals relative to saline-injected controls (FIG. 17D). As expected, Applicants saw a small population of cells that escaped transduction in the 1×10¹¹ vg dose regime with no apparent reduction in Caveolin-1 expression. By titrating dosage, experimenters could use AAV-B130 to achieve mosaic recombination—an approach which would be particularly useful to investigate the cell-autonomous function of blood-brain-barrier genes whose widespread loss throughout CNS endothelial cells is lethal, such as Claudin-5 (Nitta, T. et al. 2003), J Cell Biol 161, 653-660) or β-Catenin (Stenman, J. M. et al. (2008), Science (80). 322, 1247-1250; Liebner, S. et al. (2008), J Cell Biol 183, 409-417; Daneman, R. et al. (2009), Proc Natl Acad Sci USA 106, 641-646; Tran, K. A. et al. (2016), Circulation 133, 177-186).

In addition, Applicants verified that AAV-B130-mediated Cre delivery did not drive systemic loss of endothelial Caveolin-1. Protein levels in the heart microvasculature—an organ where endothelial Caveolin-1 is known to be robustly expressed (Zhao, Y. Y. et al. (2002), Proc Natl Acad Sci USA 99, 11375-11380; Kalucka, J. et al. (2020), Cell 180, 764-779) were not appreciably altered in AAV-B130-injected animals (FIG. 17E). This ability to rapidly perform CNS-directed loss-of-function experiments may make AAV-B130-mediated Cre delivery an attractive alternative to commonly used pan-endothelial transgenic drivers, such as CDH5:Cre^{ERT2} (Wang, Y. et al. (2010), Nature 465, 483-6) or TIE2:Cre (Kisanuki, Y. Y. et al. (2001), Dev Biol 230, 230-242).

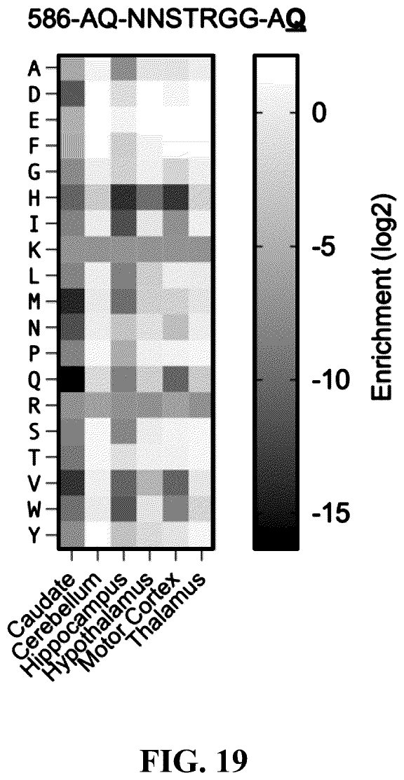

FIG. 19 shows the results of site-saturation mutagenesis of AAV-B130 at 597Q (AAV9 position 590Q) used to identify variants that outperform AAV-B130 in their ability to transduce cells in the marmoset brain. The heat map shows the mean enrichment of 10 replicates for each AAV-B130 variant in the indicated brain region. AAV-B130 Q597 to D, E, F, G, P, S, T, or Y variants are more enriched than AAV-B130 across most brain regions.

### DISCUSSION

Applicant's findings demonstrate that AAV-B130 is ideally-suited to accelerate neurovascular research, providing a rapid and easily-adaptable means to access CNS endothelial cells with clear advantages over existing vectors (Table 8). Unlike AAV-PHP.V1, at a 1×10¹¹ vg dose AAV-BI30's tropism within the CNS is almost exclusively limited to endothelial cells, obviating the need for complex workarounds—such as intersectional Cre-dependent approaches—to restrict cell-type specificity. And compared to AAV-BR1, AAV-B130 is more efficient and versatile, with particularly evident benefits for applications targeting retina, spinal cord, or cerebrovascular arteries and veins.

TABLE 8

| | | | | | | Enhanced | |
|---|---|---|---|---|---|---|---|
| | | | | | Enhanced | Transduction | |
| | | | SEQ | Dominant | Transduction | In Human | |
| | Parental | Peptide | ID | CNS | in BABLc/J | Endothelial | |
| Capsid | Vector | Insertion | NO: | Tropism | Strain? | Cell Lines? | Reference |
| BI30 | AAV9 | NNSTRGG | 1 | Endothelial Cells | Yes | Yes-(hBMVECs | |
| | | | | | | & hCMEC/D3) | |
| PHP.V1 | AAV9 | TALKPFL | 1100 | Endothelial Cells, | No | Yes-(HBMEC) | [1] |
| | | | | Astrocytes | | | |
| PHP.V2 | AAV9 | TTLKPFL | 1101 | Endothelial Cells, | Unknown | Unknown | [1] |
| | | | | Astrocytes | | | |
| PHP.eB | AAV- | [DG]TLAVPFK | 1102 | Neurons, | No | No-(hBMVECs | [2] |
| | PHP.B | | | Astrocytes, | | & hCMEC/D3) | |
| | | | | Oligodendrocytes, | | | |
| | | | | Endothelial Cells | | | |
| PHP.B | AAV9 | TLAVPFK | 1103 | Neurons, | No | Unknown | [3] |
| | | | | Astrocytes, | | | |
| | | | | Oligodendrocytes, | | | |
| | | | | Endothelial Cells | | | |
| BR1 | AAV2 | NRGTEWD | 1104 | Endothelial Cells | Unknown | No-(hCMEC/D3) | [4] |
| PPS | AAV2 | DSPAHPS | 1105 | Endothelial Cells | Unknown | No-(hCMEC/D3) | [5] |

References
[1] S. R. Kumar et al., “Multiplexed Cre-Dependent Selection Yields Systemic AAVs for Targeting
Distinct Brain Cell Types,” Nat Methods, vol. 17, pp. 541-550, 2020.
[2] K. Y. Chan et al., “Engineered AAVs for Efficient Noninvasive Gene Delivery to the Central
and Peripheral Nervous Systems,” Nat Neurosci, vol. 20, no. 8, pp. 1172-1179, 2017.
[3] B. E. Deverman et al., “Cre-Dependent Selection Yields AAV Variants for Widespread Gene
Transfer to the Adult Brain,” Nat Biotechnol, vol. 34, no. 2, pp. 204-209, 2016.
[4] J. Korbelin et al., “A Brain Microvasculature Endothelial Cell-Specific Viral Vector With
the Potential to Treat Neurovascular and Neurological Diseases,” EMBO Mol Med, vol. 8, no. 6,
pp. 609-25, 2016.
[5] Y. H. Chen, M. Chang, and B. L. Davidson, “Molecular Signatures of Disease Brain Endothelia
Provide New Sites for CNS-Directed Enzyme Therapy,” Nat Med, vol. 15, no 10, pp. 1215-1219, 2009.

While AAV-B1I30's tropism appears largely biased towards the CNS vasculature, Applicants observed transduction of liver hepatocytes and endothelial cells in the lung microvasculature, aorta, and interlobular vessels of the kidney. Importantly, peripheral endothelial transduction was restricted to these populations—most organs had limited endothelial cell transduction. For most research applications, AAV-B1I30's advantageous properties easily outweigh this caveat. However, in cases where CNS-specificity is critical, a Cre-dependent viral genome could be used in tandem with a CNS endothelial cell-specific transgenic driver—such as MFSD2A:Cre^{ERT2} (Pu, W. et al. (2016), Nat Commun 7, 13369) or SLCO1C1:Cre^{ERT2} (Ridder, D. A. et al. (2011), J Exp Med 208, 2615-2623), to minimize AAV-B1I30's peripheral transduction.

During our characterization of AAV-B130 in vivo, Applicants encountered unexpected dose-dependent hepatoxicity. By incorporating a hepatocyte-specific microRNA binding site into our viral genome, Applicants were able to effectively rescue toxicity without compromising the vector's on-target transduction efficiency. This microRNA-based strategy functions by suppressing translation of the AAV transgene in hepatocytes without affecting stages of transduction involving the capsid protein. As a result, the strategy's efficacy strongly argues against the possibility that the AAV-B130 capsid's modifications have resulted in de novo immunogenicity. Instead, the hepatotoxicity observed in our study most likely stems from cytotoxic protein overexpression of the viral transgene, a previously-characterized phenomenon (Sawada, Y. et al. (2010), Cerebellum 9, 291-302; Khabou, H., (2018), Hum Gene Ther 29, 1235-1241). Future engineering efforts could target additional surface features of the AAV-B130 capsid to limit hepatocyte transduction, similar to a recent study which generated a variant of the AAV-PHP.eB capsid de-targeted from peripheral organs (Goertsen, D. et al. (2021), Nat Neurosci doi:10.1038/s41593-021-00969-4). Alternatively, the development of robust endothelial-specific promoters would likely obviate the need to include microRNA binding sites in the viral genome. This latter approach could even be used to restrict transgene expression to one or more of the endothelial subpopulations transduced by AAV-B130 in a manner similar to vessel segment-specific transgenic drivers like BMX.Cre^{ERT2} (Ehling, M., et al. (2013), Development 140, 3051-61).

In contrast with a number of recently-discovered AAV vectors—including AAV-PHP.B (Hordeaux, J. et al. (2018), Mol Ther 26, 664-668) and AAV-PHP.V1 (Kumar, S. R. et al. (2020), Nat Methods 17, 541-550), AAV-B1I30's transduction profile was broadly similar between C57BL/6 and BALB/cJ mouse strains. Because the BALB/cJ strain's hypomorphic Ly6a allele has been directly linked to impaired CNS transduction by capsids of the AAV-PHP.B family following systemic administration (Huang, Q. et al. (2019), PLoS One 14, e0225206; Hordeaux, J. et al. (2019), Mol Ther 27, 912-921; Batista, A. R. et al. (2020), Hum Gene Ther 31, 90-102), this finding indicates that AAV-B1I30's mechanism of transduction is LY6A-independent. The apparent absence of a Ly6a homolog conserved across mammalian species (Loughner, C. L. et al. (2016), Hum Genomics 10, 10) has impeded deployment of engineered capsids in genetically intractable model organisms (Hordeaux, J. et al. (2018), Mol Ther 26, 664-668; Matsuzaki, Y. et al. (2018), Neurosci Lett 665, 182-188). Consistent with a LY6A-independent mechanism, Applicants found AAV-B130 efficiently transduced rat CNS endothelial cells in vivo and human cell lines in vitro. These results suggest that the vector may enable CNS endothelial cell transduction in a wide range of mouse strains and mammalian species. While Applicants have not yet delineated AAV-B1I30's mechanism of action, based on its tropism Applicants speculate that the virus may bind or enter cells using a surface receptor expressed on endothelial cells throughout the body but enriched in CNS and lung microvasculature. Moving forward, recent efforts to profile the transcriptomes of endothelial cells isolated from a wide variety of murine organs (Kalucka, J. et al. (2020), Cell 180, 764-779) could be leveraged to identify candidate receptors.

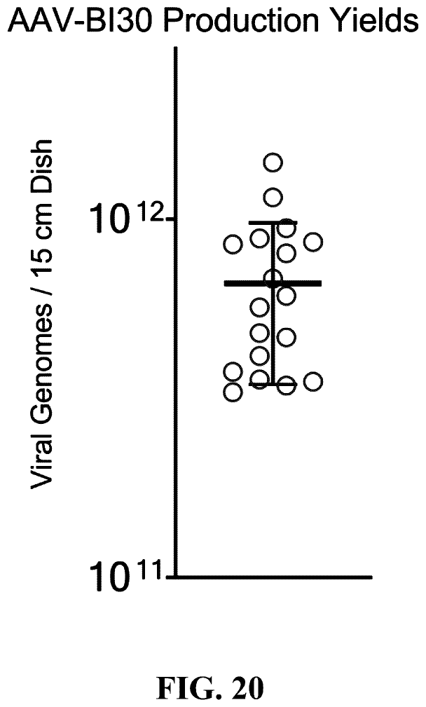

AAV-B1I30's high efficacy is likely understated by the binary quantification metric used throughout our analyses. Throughout brain, retina, and spinal cord Applicants observed a wide range of NLS-GFP intensities, suggesting that at a relatively low 1×10¹¹ vg dose Applicants delivered multiple viral genomes to a large number of CNS endothelial cells. As a result, AAV-BI30 appears exceptionally well-suited for a range of applications that require the delivery of multiple viral genomes to individual cells such as gene editing (Tabebordbar, M. et al. (2016), Science (80-.). 351, 407-411; Levy, J. M. et al. (2020), Nat BiomedEng 4, 97-110), live-imaging sensors, loss- and gain-of-function studies, or two-component tunable expression systems (Chan, K. Y. et al. (2017), Nat Neurosci 20, 1172-1179). Moreover, given that structural modifications to AAV capsids can dramatically reduce their manufacturability, it is important to note that when used to package a variety of genomes for this study—fluorescent reporters, bioluminescence producing enzymes, and gene editors, i.e., post-purification AAV-B130 yields were in line with other engineered and natural AAV vectors (FIG. 20). This versatility, combined with the capsid's conserved tropism across mouse strains and mammalian species, highlight AAV-B1I30's potential as a powerful tool to access CNS endothelial cells in vivo and catalyze our growing understanding of neurovascular processes in health and disease.

### Materials and Methods

### Animals

All procedures were approved by the Harvard University and Broad Institute of MIT and Harvard Institutional Animal Care and Use Committees (IACUC). The following commercially available mouse strains were used: C57BL/6NCrl (Charles River 027), C57BL/6J (Jackson Laboratory 000664), BALB/cJ (Jackson Laboratory 000651), and Ai9 (Jackson Laboratory 007909; originally generated by Madisen et al. (Madisen, L. et al. (2010), Nat Neurosci 13, 133-140)). CD® (Sprague Dawley) IGS rat was obtained from Charles River (Strain Code 001).

Cav1^{flox} mice were originally generated by Asterholm et al. (Asterholm, I. et al. (2012), Cell Metab 15, 171-185) and generously shared by Philipp Scherer. The line was genotyped using Phire Green Hot Start II DNA Polymerase (Thermo Fischer F124L) and the following primers: 5′-GTGCATCAGCCGCGTCTACTCC-3′ (SEQ ID NO: 1106) and 5′-GGCCGTAACCTGAATCTCTTCCCTTTG-3′ (SEQ ID NO: 1107). PCR reaction with genomic DNA samples produces ˜490 bp wild-type and ˜445 bp floxed products.

Recombinant AAV vectors were administered intravenously via the tail vein or retro-orbital sinus in young adult male or female animals. Mice were randomly assigned to groups based on predetermined sample sizes. No mice were excluded from the analyses. Experimenters were not blinded to sample groups.

### Virus Production and Titering

Recombinant AAVs were generated by triple transfection of HEK293T cells using polyethylenimine (PEI) and purified by ultracentrifugation over iodixanol gradients as previously described (Batista, A. R. et al. (2020), Hum Gene Ther 31, 90-102). Purified virus was incubated with 1000U/mL Turbonuclease (Sigma T4330-50KU) with 1× DNase I reaction buffer (NEB B0303S) at 37° C. for one hour. The endonuclease solution was inactivated with 0.5M, pH 8.0 EDTA at room temperature for 5 minutes and then at 70° C. for 10 minutes. AAV genomes were released by incubation with 100 g/mL Proteinase K (Qiagen, 19131) in 1M NaCl, 1% N-lauroylsarcosine, and in UltraPure DNase/RNase-Free water at 56° C. for 2 to 16 hours before heat inactivation at 95° C. for 10 minutes. The nuclease-resistant AAV genomes were diluted between 460-460,000× and 2 μL of the diluted samples were used as input in a ddPCR supermix for probes (Bio-Rad, 1863023) with 900 nM ITR2_Forward (5′-GGAACCCCTAGTGATGGAGTT-3′ (SEQ ID NO: 1108)), 900 nM ITR2_Reverse (5′-CGGCCTCAGTGAGCGA-3′(SEQ ID NO: 1109)), and 250 nM ITR2_Probe (5′-HEX-CACTCCCTC-ZEN-TCTGCGCGCTCG-IABkFQ-3′ (SEQ ID NO: 1110)). The ITR2_Probe contained the following modifications—5′ HEX dye, ZEN internal quencher, and 3′ Iowa Black fluorescent quencher (IDT, PrimeTime qPCR Probes). Droplets were generated using a QX100 Droplet Generator, transferred to thermocycler, and cycled according to the manufacturer's protocol with an annealing/extension of 58° C. for 1 minute. Finally, droplets were read on a QX100 Droplet Digital System to determine titers.

### In Vitro Transduction Assays

Multiple lots of mBMVEC and hBMVEC cells were obtained from CellBiologics (H-6023 & C57-6023) and maintained in endothelial cell media (H1168 & M1168). hCMEC/D3 cells were obtained from Millipore (SCC0066) and maintained in EndoGRO™-MV Complete Media (SCME004). All cells were handled according to the manufacturer's instructions. For the Luciferase assays, 5000 cells/well were seeded in 96 well plates (PerkinElmer, 6005680). One day later, AAV9, AAV-PHP.eB or AAV-B130 carrying pAAV-CAG-eGFP-p2A-luciferase was added at 20,000 vg/cell. 24 hours after transduction, a luciferase reporter assay was performed according to the manufacturer's instructions (PerkinElmer, 6066761) on an EnSpire plate reader (PerkinElmer). For flow cytometry, hCMEC/D3 cells were plated at 434,000 cells/well in 24 well plates and exposed to the indicated dose of AAV-B130 or AAV9. The media was exchanged for fresh media after 24 hours and transduction was assessed at 4 days post-administration on a Beckman CytoFLEX S Flow Cytometer.

### Capsid Library Generation

The mRNA selection vector (AAV9-CMV-Express) was designed to enrich for functional AAV capsid sequences by recovering capsid mRNA from transduced cells. AAV9-CMV-Express uses a ubiquitous CMV enhancer and AAV5 p41 gene regulatory elements to drive AAV9 Cap expression. The AAV9-Express plasmid was constructed by cloning the following elements into an AAV genome plasmid in the listed order: a cytomegalovirus (CMV) enhancer-promoter, a synthetic intron, and the AAV5 P41 promoter along with the 3′ end of the AAV2 Rep gene, which includes the splice donor sequences for the capsid RNA. The capsid gene splice donor sequence in AAV2 Rep was modified to a consensus donor sequence CAGGTAAGT. The AAV9 capsid gene sequence was synthesized with nucleotide changes at 1344, 1346, and 1347 (which introduces a K449R mutation) and at 1782 (which is a silent mutation) to introduce restriction enzyme recognition sites for NNK library PCR insert fragment cloning. The AAV2 polyadenylation sequence was replaced with a simian virus 40 (SV40) late polyadenylation signal.

The initial random 7-mer library was produced using 5′-CGGACTCAGACTATCAGCTCCC-3′ (SEQ ID NO: 1111) and 5′-GTATTCCTTGGTTTTGAACCCAACCGGTCTGCGCCTGTGCMNNMNNMNNMNNMNN MNNMNNTTGGGCACTCTGGTGGTTTGTG-3′ (SEQ ID NO: 1112) primers (IDT) to PCR amplify a modified AAV9 template (K449R) using Q5 Hot Start High-Fidelity 2× Master Mix (NEB, M0494S) following the manufacturer's protocol. The PCR products were cleaned up using AMPure XP beads (Beckman, A63881) following the manufacturer's protocol. The NNK PCR insert was assembled into a linearized mRNA selection vector (AAV9-CMV-Express) with NEBuilder HiFi DNA Assembly Master Mix (NEB, E2621L). Afterwards, Quick CIP (NEB, M0508S) was spiked into the reaction and incubated at 37° C. for 30 minutes to dephosphorylate unincorporated dNTPs. Finally, T5 Exonuclease (NEB M0663 S) was added to the reaction mixture and incubated at 37° C. for 30 minutes to remove unassembled products. The final assembled product was cleaned up using AMPure XP beads (Beckman, A63881) following the manufacturer's protocol quantified using the Qubit dsDNA HS Assay Kit (ThermoFisher Scientific, Q32851). Capsid variants chosen for secondary screening were synthesized as an oligopool (Agilent). Each capsid 7-mer was represented by two unique nucleotide sequences and cloned into the AAV9-CMV-Express backbone as described above.

### Capsid Library Screening

For in vivo screening, the AAV capsid library was injected at 1×10¹¹ vg/animal in either C57BL/6J or BALB/cJ mice. 21 days post-injection animals were culled, and tissue was extracted and flash-frozen for RNA isolation. For in vitro screening, 1×10¹¹ vg of the AAV capsid library was added to confluent BMVECs or hCMEC/D3 cells and cellular mRNA was collected 3 days post-administration. mRNA from in vivo and in vitro assays were recovered using TRIzol (Invitrogen, 15596026) followed by RNA cleanup with RNeasy Mini Kit (Qiagen, 74104). The recovered mRNA was next converted to cDNA using an oligo dT primer using Maxima H Minus Reverse Transcriptase (ThermoFisher, EP0751). To prepare AAV libraries for sequencing, qPCR was performed on the converted cDNA from each sample type to identify the minimum number of cycles necessary for amplification. Once cycle thresholds were determined SeqF1 (5′-CTTTCCCTACACGACGCTCTTCCGATCTNCCAACGAAGAAGAAATTAAAACTACTAA CCCG-3′ (SEQ ID NO: 1113)) and SeqR1 (5′-GGAGTTCAGACGTGTGCTCTTCCGATCTCATCTCTGTCCTGCCAAACCATACC-3′ (SEQ ID NO: 1114)) primers were used to attach Illumina Read 1 and Read 2 sequences using Q5 Hot Start High-Fidelity 2× Master Mix with an annealing temperature of 65° C. for 20 seconds and an extension time of 1 minute with a cycle number determined from the qPCR. These PCR products were purified using AMPure XP beads following the manufacturer's protocol and eluted in 25 μL UltraPure Water (ThermoScientific) and then 2 μL was used as input in a secondary PCR to attach Illumina adaptors and dual index primers (NEB, E7600S) for 5 cycles using Q5 HotStart-High-Fidelity 2× Master Mix with an annealing temperature of 65° C. for 20 seconds and an extension time of 1 minute. The second PCR products were purified using AMPure XP beads following the manufacturer's protocol and eluted in 25 μL UltraPure DNase/RNase-Free distilled water (ThermoScientific, 10977015). To quantify the PCR product for NGS, an Agilent High Sensitivity DNA Kit (Agilent, 5067-4626) was used with an Agilent 2100 Bioanalyzer system. The secondary PCR products were then pooled and diluted to 2-4 nM in 10 mM Tris-HCl, pH 8.5 and sequenced on an Illumina NextSeq 550 following the manufacturer's instructions using a NextSeq 500/550 Mid or High Output Kit (Illumina, 20024904 or 20024907). Reads were allocated as follows: 1l: 8, 12: 8, R1: 150, R2: 0.

### Cloning

The AAV-B130 rep-cap plasmid was generated by assembling an oligo (IDT) containing the AAV-B130 DNA sequence (5′-AACAACTCAACCCGCGGCGGC-3′ (SEQ ID NO: 1115)) into a synthesized kanamycin resistant rep-tTA-Cap helper (iCapK2-BI30; GenScript) containing a K449R point mutation in AAV9. The AAV-BR1 cap gene was synthesized and cloned into the same iCapK2 backbone. To generate pAAV-CAG-NLS-GFP-miR122-WPRE and pAAV-CAG-Cre-miR122-WPRE gene fragments were synthesized (GenScript) and cloned into pAAV-CAG-mTurquoise2, a gift from Viviana Gradinaru (Addgene #99122).

### Immunohistochemistry

Mice were deeply anesthetized with an intraperitoneal injection of a ketamine/xylazine solution and transcardially perfused with ˜15 mL of room-temperature PBS followed by ˜20 mL of ice-cold 4% PFA using a peristaltic pump set to a flow rate of ˜9 mL/min. Tissues were subsequently harvested and processed as follows: Pia Vasculature Whole Mounts. Brain was dissected out of the skull and partially immersed in PBS in a glass dish. A razor blade was used to make a cut along the sagittal midline followed by a cut along the horizontal axis to separate each hemisphere into dorsal and ventral pieces. Dorsal-facing brain samples were then transferred into a 48-well plate and post-fixed in 4% PFA on ice for 30 minutes. All subsequent wash steps were carried out in the plate, and care was taken to ensure the ventral surface of the brain sample always remained in contact with the bottom of the dish. Samples were washed 3× with PBS, blocked with a 10% Donkey Serum/0.5% PBST (Triton X-100) solution for 2 hours at room temperature, and then incubated with primary antibodies made up in blocking solution for 48 hours at 4° C. with agitation. Next, samples were washed 3× with 0.5% PBST and 1× with PBS. To capture images, brain sample was placed dorsal side down in a 2-well glass bottom slide (Ibidi 80287) partially filled with PBS such that the pia vasculature faced the objective on an inverted microscope. Retina Whole Mounts. Eyes were removed from the eye sockets and briefly post-fixed in room-temperature 4% PFA for 5 minutes. Next, retinas were isolated via fine dissection in PBS and further post-fixed in room-temperature 4% PFA for 30 minutes. Retinas were washed 3× with PBS, blocked with a 10% Donkey Serum/0.5% PBST solution for 1 hour at room temperature, and then incubated with primary antibodies made up in blocking solution overnight at 4° C. with agitation. Next, retinas were washed 3× with 0.5% PBST, 1× with PBS, and flat-mounted on glass coverslips. Aorta Whole Mounts. Thoracic aorta was grossly dissected and immersed in PBS. Fat, connective tissue, and arterial branches were subsequently removed via fine dissection. A post-fixation step was omitted. Aortas were washed 3× with PBS, blocked with a 10% Donkey Serum/0.5% PBST solution for 1 hour at room temperature, and then incubated with primary antibodies made up in blocking solution overnight at 4° C. with agitation. Next, aortas were washed 3× with 0.5% PBST and immersed in PBS. A single cut was made lengthwise along the vessels to expose the lumen and the aortas were flat-mounted enface on glass coverslips. Tissue Sections. Organs were post-fixed in 4% PFA at 4° C. overnight. Alternatively, brain was post-fixed 4 hours in 4% PFA on ice for Caveolin-1 immunostaining due to fixation-sensitivity of the antibody. Samples were then washed 3× with PBS to remove residual PFA. For cryosections, samples were cryopreserved in a 30% sucrose solution at 4° C. overnight, and then frozen in NEG-50 (Richard-Allan Scientific 6502). 18 μm (FIG. 4F; FIG. 15D; FIG. 17D; FIG. 9) or 30 μm (FIG. 1C & E [BALB/cJ]; FIGS. 17B, 17C and 17E; FIG. 5, FIG. 7, FIG. 8 [BALB/cJ], FIG. 10, FIG. 12) sections were cut using a cryostat. For vibratome sections, 60 μm (FIGS. 4B & 4E [rat]; FIG. 3, FIG. 8 [rat]) or 100 μm (FIG. 4D; FIG. 17A) sections were cut using a LeicaVT1200S vibratome. Sections were washed 3× in PBS, permeabilized in 0.5% PBST for 10 minutes, and then blocked with a 5% Donkey Serum/0.1% PBST solution for 1 hour at room temperature. Sections were subsequently incubated with primary antibodies made up in blocking solution overnight at 4° C., washed 3× with 0.1% PBST, and incubated with secondary antibodies and DAPI made up in blocking solution for 1 hour at room temperature. Finally, sections were washed 3× with 0.1% PBST, 1× with PBS, and coverslipped for imaging.

The following primary antibodies were used at the indicated concentrations: Rabbit anti-ERGºAlexa Fluor 647 (1:100; Abcam ab196149), Rabbit anti-GFPºAlexa Fluor 488 (1:200; Thermo Fisher Scientific A-21311), Mouse anti-α Smooth Muscle Actin—Cy3 (1:500; Sigma-Aldrich C6198), Mouse anti-α Smooth Muscle ActinºAlexa Fluor 647 (1:100; Santa Cruz Biotechnology sc-32251), Goat anti-CD31 (1:100; R&D Systems AF3628), Rat anti-ICAM2 (1:100; BD Biosciences 553326), Rabbit anti-Caveolin-1 (1:200; Cell Signaling Technology 3267), Mouse anti-RECA-1 (1:200; Abcam ab9774), and Chicken anti-GFP (1:1000; Aves Labs GFP-1020). In addition, Isolectin GS-1B4ºAlexa Fluor 568 (1:100; Invitrogen 121412) was used to stain vasculature in retina whole mounts. Specificity of the monoclonal anti-Cav1 antibody used in this study has been previously demonstrated by our group in Caveolin-1 knockout mice (Chow, B. W. et al. (2020), Nature 579, 106-110).

Donkey anti-Goat IgGºAF488 (1:250; Jackson ImmunoResearch 705-545-003), Donkey anti-Goat IgGºCy3 (1:250; Jackson ImmunoResearch 705-165-147), Donkey anti-Rat IgGºAF488 pre-adsorbed against Mouse IgG (1:250; Jackson ImmunoResearch 712-546-153), Donkey anti-Rat IgGºCy3 pre-adsorbed against Mouse IgG (1:250; Jackson ImmunoResearch 712-165-153), Donkey anti-Rabbit IgGºAF647 (1:250; Jackson ImmunoResearch 711-605-152), Donkey anti-Mouse IgGºAF546 highly cross-adsorbed (1:1000; Invitrogen A10036), and Goat anti-ChickenºAF488 (1:1000 Invitrogen, A-11039) were used in conjunction with unconjugated primary antibodies.

Representative images were acquired with a Leica TCS SP8 confocal microscope, a Keyence BZ-X810, or a Nikon Ti-E inverted microscope/Andor CSU-X1 spinning disc confocal with an Andor DU-888 EMCCD camera.

### Quantification of Viral Transduction Efficiency

### NLS-GFP Overexpression

Cell Profiler (McQuin, C. et al. (2018), PLoS Biol 16, e2005970) was used to construct unbiased, semi-automated or fully-automated analysis pipelines for all tissues examined. Viral transduction was assessed using a binary metric: GFP⁺ERG⁺ cells taken as a fraction of total ERG⁺ cells. Staining and imaging of AAV-B130 and AAV-BR1 samples was performed on the same day to ensure identical imaging conditions. All quantification was performed in C57BL/6NCrl mice. Retina, spinal cord, and brain data were collected from the same set of animals. Tissue-specific workflow was as follows: Brain Microvasculature. A single sagittal brain section was acquired from each animal and imaged in its entirety using an Olympus VS120 whole slide-scanning microscope with a UPLSAPO 20×/0.75 objective lens. An average of 15,310±3,929 (mean±s.d.) endothelial cells were identified by the analysis pipeline in each replicate. Pia Vasculature. Three ˜1164 μm×1164 μm FOVs of the pia vasculature were acquired for each animal using a Leica TCS SP8 confocal microscope with a HC PL APO 10×/0.40 objective lens. Relatively flat regions of the brain surface were sampled with shallow z-stacks to clearly separate pia vasculature from the underlying capillary plexus. Arteries and veins were manually identified using (i) the presence of α-Smooth Muscle Actin (high in arteries, low-to-absent in veins) and (ii) the nuclear morphology of endothelial cells (ellipsoidal in arteries, circular in veins). Artery-vein overlap regions were omitted from analysis. Average endothelial cell counts identified by analysis pipeline per animal were as follows: 1,197±182 (aECs), 1,908±197 (vECs). Retina Vasculature. Organization of the retinal vasculature is highly stereotyped; vessels form three distinct vascular plexuses. Blood enters via the retinal artery which emerges alongside the optic nerve, branching into spoke-like radial arteries that spread across the superficial plexus. These arteries elaborate to form a dense capillary network that constitute the majority of the intermediate and deep plexuses. Capillaries in the deep plexus finally merge into draining venules which ascend to the superficial plexus and form veins interleaved among the retina's arteries. To quantify viral transduction efficiency across the entirety of the retinal arterio-venous axis, four sub-regions of each retina whole-mount were sampled: radial arteries in the superficial plexus; capillaries in the intermediate plexus; capillaries in the deep plexus; and radial veins in the superficial plexus. Three ˜290 μm×290 μm FOVs of each of these vascular sub-regions were acquired for each animal using a Leica TCS SP8 confocal microscope with a HC PL APO CS2 20×/0.75 objective lens. Special care was taken when setting the z-axis bounds on each image in order to isolate signal from a single vascular plexus. Superficial plexus arteries and veins within a given FOV were manually identified using SMA expression and nuclear morphology as described for pia vasculature. All ERG⁺ nuclei within intermediate and deep plexus FOVs were counted—the vast majority of these endothelial cells belong to capillary microvessels. Average endothelial cell counts identified by analysis pipeline per animal were as follows: 155±12 (superficial plexus aECs), 157±13 (intermediate plexus ECs), 229±19 (deep plexus ECs), 102±11 (superficial plexus vECs). Spinal Cord Microvasculature. Imaging was performed using a whole-slide scanning microscope as described for the brain microvasculature. Images of two transverse sections were acquired for each animal and used to identify an average of 1,061±200 endothelial cells in each replicate. Non-Endothelial Transduction. (Fiji is Just) ImageJ's Cell Counter plugin was used to manually quantify non-endothelial transduction in cortex. These counts were collected from the aforementioned sagittal sections of brain used to quantify endothelial transduction efficiency in brain microvasculature (18 μm thick; imaged with an Olympus VS120 whole slide-scanning microscope). DAPI⁺GFP⁺ERG-nuclei were identified as instances of non-endothelial transduction. Ai9 Recombination Efficiency. Three ˜582 m×582 μm FOVs of the cortical microvasculature per replicate were acquired from 30 μm sagittal sections using a Leica TCS SP8 confocal microscope with a HC PL APO CS2 20×/0.75 objective lens. A modified Cell Profiler pipeline similar to those used to quantify AAV-mediated NLS-GFP overexpression was employed to count tdT⁺ ERG⁺ cells taken as a fraction of total ERG⁺ cells. An average of 481±73 endothelial cells were identified by the analysis pipeline per replicate.

### Cranial Window Implantation and Two-Photon Imaging

The cranial window implantation workflow was based on the protocol described by Goldey et al. (Goldey, G. J. et al. (2014), Nat Protoc 9, 2515-2538). The craniotomy was centered over somatosensory cortex at approximately 2 mm posterior and 2.5 mm lateral to bregma. The perimeter of the craniotomy was traced using a 4 mm circular biopsy punch (VWR 21909-140) marked with a surgical marker (Aspen Surgical 1000-00-PDG). A custom titanium headplate was then centered on this trace and bonded to the skull with dental cement (C&B Metabond; Parkell S396, S398, S371). Next, a micro-motor drill (Foredom, MH-170) outfitted with a 0.2 mm miniature carbide burr bit (Stoelting, 51451) was used to carefully remove the bone along the circumference of the craniotomy trace. The resulting circular bone flap was subsequently removed with fine forceps while continuously irrigating with saline so as to avoid damage to the pia vasculature. A cranial window—composed of a 4 mm round cover glass (Warner Instruments CS-4, 64-0724) glued to a 5 mm round cover glass (Warner Instruments CS-5R, 64-0700) with a UV-curable adhesive (Norland Products NOA68)—was carefully lowered onto the exposed brain and bonded to surrounding regions of the skull with dental cement. Finally, a well-composed of 0-Rings (USA Sealing, ZUSAH1X10 & ZUSAH1X10.5) adhered to one another with cyanoacrylate (3M Vetbond)—was constructed around the window to facilitate use of a water-immersion objective. Immediately prior to imaging, 1 mg of 70 kDa Dextran—Texas Red (Thermo Fisher D1830) was dissolved in 75 μL sterile saline and injected intravenously via the tail vein to visualize the vasculature. In vivo imaging was performed with a previously described custom-built two photon microscope (Chow, B. W. et al. (2020), Nature 579, 106-110). using a Mai Tai Ti:Sapphire (Spectra-Physics) laser tuned to 900 nm.

### Image Processing and Data Analysis

ImageJ and Adobe Illustrator were used to process images displayed in figures throughout the manuscript. In cases where AAV-B130 and AAV2-BR1 were directly compared, matched images were treated identically. GraphPad Prism 9.1.1 was used for statistical analysis and graph generation.

### REFERENCES

Deverman, B. E. et al. Cre-Dependent Selection Yields AAV Variants for Widespread Gene Transfer to the Adult Brain. Nat Biotechnol 34, 204-209 (2016).
Körbelin, J. et al. A Brain Microvasculature Endothelial Cell-Specific Viral Vector With the Potential to Treat Neurovascular and Neurological Diseases. EMBO Mol Med 8, 609-25 (2016).
Tervo, D. G. R. et al. A Designer AAV Variant Permits Efficient Retrograde Access to Projection Neurons. Neuron 92, 372-382 (2016).
Chan, K. Y. et al. Engineered AAVs for Efficient Noninvasive Gene Delivery to the Central and Peripheral Nervous Systems. Nat Neurosci 20, 1172-1179 (2017).
Hanlon, K. S. et al. Selection of an Efficient AAV Vector for Robust CNS Transgene Expression. Mol Ther Methods Clin Dev 15, 320-332 (2019).
Kumar, S. R. et al. Multiplexed Cre-Dependent Selection Yields Systemic AAVs for Targeting Distinct Brain Cell Yypes. Nat Methods 17, 541-550 (2020).
Nonnenmacher, M. et al. Rapid Evolution of Blood-Brain-Barrier-Penetrating AAV Capsids by RNA-Driven Biopanning. Mol Ther Methods Cin Dev 20, 366-378 (2020).
Sweeney, M. D., Kisler, K., Montagne, A., Toga, A. W. & Zlokovic, B. V. The Role of Brain Vasculature in Neurodegenerative Disorders. Nat Neurosci 21, 1318-1331 (2018).
Mastorakos, P. & McGavern, D. The Anatomy and Immunology of Vasculature in the Central Nervous System. Sci Immunol 4, eaav0492 (2019).
Vanlandewijck, M. et al. A Molecular Atlas of Cell Types and Zonation in the Brain Vasculature. Nature 554, 475-480 (2018).
Chen, B. R., Kozberg, M. G., Bouchard, M. B., Shaik, M. A. & Hillman, E. M. A Critical Role for the Vascular Endothelium in Functional Neurovascular Coupling in the Brain. J Am Hear. Assoc 3, e000787 (2014).
Longden, T. A. et al. Capillary K+—Sensing Initiates Retrograde Hyperpolarization to Increase Local Cerebral Blood Flow. Nat Neurosci 20, 717-726 (2017).
Chow, B. W. et al. Caveolae in CNS Arterioles Mediate Neurovascular Coupling. Nature 579, 106-110 (2020).
Ben-Zvi, A. et al. Mfsd2a is Critical for the Formation and Function of the Blood-Brain Barrier. Nature 509, 507-511 (2014).
Andreone, B. J. et al. Blood-Brain Barrier Permeability Is Regulated by Lipid Transport-Dependent Suppression of Caveolae-Mediated Transcytosis. Neuron 94, 581-594 (2017).
Chow, B. W. & Gu, C. Gradual Suppression of Transcytosis Governs Functional Blood-Retinal Barrier Formation. Neuron 93, 1325-1333.e3 (2017).
Kerfoot, S. M. & Kubes, P. Overlapping Roles of P-Selectin and Alpha 4 Integrin to Recruit Leukocytes to the Central Nervous System in Experimental Autoimmune Encephalomyelitis. J Immunol 169, 1000-1006 (2002).
Piccio, L. et al. Molecular Mechanisms Involved in Lymphocyte Recruitment in Inflamed Brain Microvessels: Critical Roles for P-Selectin Glycoprotein Ligand-1 and Heterotrimeric G(i)-Linked Receptors. J Immunol 168, 1940-1949 (2002).
Bartanusz, V., Jezova, D., Alajajian, B. & Digicaylioglu, M. The Blood-Spinal Cord Barrier: Morphology and Clinical Implications. Ann Neurol 70, 194-206 (2011).
Stahl, A. et al. The Mouse Retina as an Angiogenesis Model. Invest Ophthalmol Vis Sci 51, 2813-2826 (2010).
Newman, E. A. Functional Hyperemia and Mechanisms of Neurovascular Coupling in the Retinal Vasculature. J Cereb Blood Flow Metab 33, 1685-1695 (2013).
Tan, C. et al. Endothelium-Derived Semaphorin 3G Regulates Hippocampal Synaptic Structure and Plasticity via Neuropilin-2/PlexinA4. Neuron 101, 920-937.e13 (2019).
Santisteban, M. M. et al. Endothelium-Macrophage Crosstalk Mediates Blood-Brain Barrier Dysfunction in Hypertension. Hypertension 76, 795-807 (2020).
Liu, X. xiu et al. Endothelial Cdk5 Deficit Leads to the Development of Spontaneous Epilepsy Through CXCL1/CXCR2-Mediated Reactive Astrogliosis. JExpMed 217, (2020).
Dogbevia, G., Grasshoff, H., Othman, A., Penno, A. & Schwaninger, M. Brain Endothelial Specific Gene Therapy Improves Experimental Sandhoff Disease. J Cereb Blood Flow Metab 40, 1338-1350 (2020).
Chen, D. Y. et al. Endothelium-Derived Semaphorin 3G Attenuates Ischemic Retinopathy by Coordinating β-Catenin-Dependent Vascular Remodeling. J Clin Invest 131, e135296 (2021).
Nikolakopoulou, A. M. et al. Endothelial LRP1 Protects Against Neurodegeneration By Blocking Cyclophilin A. JExp Med 218, e20202207 (2021).
Cui, Y. et al. Brain Endothelial PTEN/AKT/NEDD4-2/MFSD2A Axis Regulates Blood-Brain Barrier Permeability. Cell Rep. 36, 109327 (2021).
Song, X. et al. Genome Editing with AAV-BR1-CRISPR in Postnatal Mouse Brain Endothelial Cells. Int J Biol Sci 18, 652-660 (2022).
Tabebordbar, M. et al. Directed Evolution of a Family of AAV Capsid Variants Enabling Potent Muscle-Directed Gene Delivery Across Species. Cell 184, 4919-4938.e22 (2021).
Huang, Q. et al. Delivering Genes Across the Blood-Brain Barrier: LY6A, A Novel Cellular Receptor for AAV-PHP.B Capsids. PLoS One 14, e0225206 (2019).
Hordeaux, J. et al. The GPI-Linked Protein LY6A Drives AAV-PHP.B Transport across the Blood-Brain Barrier. Mol Ther 27, 912-921 (2019).
Lagos-Quintana, M. et al. Identification of Tissue-Specific microRNAs from Mouse. Curr Biol 12, 735-739 (2002).
Landgraf, P. et al. A Mammalian microRNA Expression Atlas Based on Small RNA Library Sequencing. Cell 129, 1401-1414 (2007).
Suzuki, T. et al. miR-122a-Regulated Expression of a Suicide Gene Prevents Hepatotoxicity Without Altering Antitumor Effects in Suicide Gene Therapy. Mol Ther 16, 1719-1726 (2008).
Xie, J. et al. MicroRNA-Regulated, Systemically Delivered rAAV9: A Step Closer to CNS-Restricted Transgene Expression. Mol Ther 19, 526-535 (2011).
Geisler, A. et al. microRNA122-Regulated Transgene Expression Increases Specificity of Cardiac Gene Transfer Upon Intravenous Delivery of AAV9 Vectors. Gene Ther 18, 199-209 (2011).
Zhang, Y. et al. An RNA-Sequencing Transcriptome and Splicing Database of Glia, Neurons, and Vascular Cells of the Cerebral Cortex. JNeurosci 34, 11929-11947 (2014).
Hordeaux, J. et al. The Neurotropic Properties of AAV-PHP.B Are Limited to C57BL/6J Mice. Mol Ther 26, 664-668 (2018).
Matsuzaki, Y. et al. Intravenous Administration of the Adeno-Associated Virus-PHP.B Capsid Fails to Upregulate Transduction Efficiency in the Marmoset Brain. Neurosci Lett 665, 182-188 (2018).
dela Paz, N. G. & D′Amore, P. A. Arterial Versus Venous Endothelial Cells. Cell Tissue Res 335, 5-16 (2008).
McQuin, C. et al. CellProfiler 3.0: Next-Generation Image Processing for Biology. PLoSBiol 16, e2005970 (2018).
Shah, A. V., Birdsey, G. M. & Randi, A. M. Regulation of Endothelial Homeostasis, Vascular Development and Angiogenesis by the Transcription Factor ERG. Vasc. Pharmacol 86, 3-13 (2016).
Graßhoff, H. et al. Short Regulatory DNA Sequences to Target Brain Endothelial Cells for Gene Therapy. J Cereb Blood Flow Metab (2021). doi:10.1177/0271678X211039617.
Hill, R. A. et al. Regional Blood Flow in the Normal and Ischemic Brain Is Controlled by Arteriolar Smooth Muscle Cell Contractility and Not by Capillary Pericytes. Neuron 87, 95-110 (2015).
Hobson, B. & Denekamp, J. Endothelial Proliferation in Tumours and Normal Tissues: Continuous Labelling Studies. Br JCancer 49, 405-413 (1984).
Madisen, L. et al. A Robust and High-Throughput Cre Reporting and Characterization System for the Whole Mouse Brain. Nat Neurosci 13, 133-140 (2010).
Fra, A. M., Williamson, E., Simons, K. & Parton, R. G. De Novo Formation of Caveolae in Lymphocytes by Expression of VIP21-Caveolin. Proc Natl Acad Sci USA 92, 8655-8659 (1995).
Razani, B. et al. Caveolin-1 Null Mice are Viable but Show Evidence of Hyperproliferative and Vascular Abnormalities. J Biol Chem 276, 38121-38138 (2001).
Sadeghian, H. et al. Spreading Depolarizations Trigger Caveolin-1-Dependent Endothelial Transcytosis. Ann Neurol 84, 409-423 (2018).
Asterholm, I. W., Mundy, D. I., Weng, J., Anderson, R. G. W. & Scherer, P. E. Altered Mitochondrial Function and Metabolic Inflexibility Associated with Loss of Caveolin-1. Cell Metab 15, 171-185 (2012).
Nitta, T. et al. Size-Selective Loosening of the Blood-Brain Barrier in Claudin-5-Deficient Mice. J Cell Biol 161, 653-660 (2003).
Stenman, J. M. et al. Canonical Wnt Signaling Regulates Organ-Specific Assembly and Differentiation of CNS Vasculature. Science (80-.). 322, 1247-1250 (2008).
Liebner, S. et al. Wnt/Beta-Catenin Signaling Controls Development of the Blood-Brain Barrier. JCell Biol 183, 409-417 (2008).
Daneman, R. et al. Wnt/0-Catenin Signaling is Required for CNS, but not Non-CNS, Angiogenesis. Proc Natl Acad Sci USA 106, 641-646 (2009).
Tran, K. A. et al. Endothelial β-Catenin Signaling Is Required for Maintaining Adult Blood-Brain Barrier Integrity and Central Nervous System Homeostasis. Circulation 133, 177-186 (2016).
Zhao, Y. Y. et al. Defects in Caveolin-1 Cause Dilated Cardiomyopathy and Pulmonary Hypertension in Knockout Mice. Proc NatlAcadSci USA 99, 11375-11380 (2002).
Kalucka, J. et al. Single-Cell Transcriptome Atlas of Murine Endothelial Cells. Cell 180, 764-779 (2020).
Wang, Y. et al. Ephrin-B2 Controls VEGF-Induced Angiogenesis and Lymphangiogenesis. Nature 465, 483-6 (2010).
Kisanuki, Y. Y. et al. Tie2-Cre Transgenic Mice: A New Model for Endothelial Cell-Lineage Analysis In Vivo. Dev Biol 230, 230-242 (2001).
Pu, W. et al. Mfsd2a+ Hepatocytes Repopulate the Liver During Injury and Regeneration. Nat Commun 7, 13369 (2016).
Ridder, D. A. et al. TAK1 in Brain Endothelial Cells Mediates Fever and Lethargy. JExpMed 208, 2615-2623 (2011).
Sawada, Y. et al. High Transgene Expression by Lentiviral Vectors Causes Maldevelopment of Purkinje Cells In Vivo. Cerebellum 9, 291-302 (2010).
Khabou, H., Cordeau, C., Pacot, L., Fisson, S. & Dalkara, D. Dosage Thresholds and Influence of Transgene Cassette in Adeno-Associated Virus-Related Toxicity. Hum Gene Ther 29, 1235-1241 (2018).
Goertsen, D. et al. AAV Capsid Variants With Brain-Wide Transgene Expression and Decreased Liver Targeting After Intravenous Delivery in Mouse and Marmoset. Nat Neurosci (2021). doi:10.1038/s41593-021-00969-4
Ehling, M., Adams, S., Benedito, R. & Adams, R. H. Notch Controls Retinal Blood Vessel Maturation and Quiescence. Development 140, 3051-61 (2013)
Batista, A. R. et al. Ly6a Differential Expression in Blood-Brain Barrier Is Responsible for Strain Specific Central Nervous System Transduction Profile of AAV-PHP.B. Hum Gene Ther 31, 90-102 (2020).
Loughner, C. L. et al. Organization, Evolution and Functions of the Human and Mouse Ly6/uPAR Family Genes. Hum Genomics 10, 10 (2016).
Tabebordbar, M. et al. In Vivo Gene Editing in Dystrophic Mouse Muscle and Muscle Stem Cells. Science (80-.). 351, 407-411 (2016).
Levy, J. M. et al. Cytosine and Adenine Base Editing of the Brain, Liver, Retina, Heart and Skeletal Muscle of Mice Via Adeno-Associated Viruses. Nat Biomed Eng 4, 97-110 (2020).
DiMattia, M. A. et al. Structural Insight into the Unique Properties of Adeno-Associated Virus Serotype 9. J Virol 86, 6947-6958 (2012).
Goldey, G. J. et al. Removable Cranial Windows for Long-Term Imaging in Awake Mice. Nat Protoc 9, 2515-2538 (2014).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A composition comprising;
a targeting moiety effective to increase transduction of endothelial cells of the CNS vasculature, the targeting moiety comprising an n-mer motif, the n-mer motif comprising or consisting of X1-N-X3-X4-X5-X6-X7, wherein X5 is independently selected from K or R, and X1, X3, X4, X6 and X7 are independently selected from any amino acid, optionally wherein the overall charge of the n-mer motif at neutral pH is between 0 and +2; and
optionally further comprising a cargo coupled to or otherwise associated with the targeting moiety.

2. The composition of claim 1, wherein X1, X3, X4, X6, and X7 is independently selected from the following groups;
X1 is selected from the group consisting of G, M, T, S, N, D, L, H, P, I, V, Q, Y, W, F, A, E;
X3 is selected from N, S, T, H, D, A, Y, M, Q, E, R, G, V;
X4 is selected from T, V, I, A, M, S, H, W, N;
X6 is selected from N, S, G, D, P, T, H, Q, A, Y;
X7 is selected from T, Y, W, N, V, I, H, M, S, G, A, Q, F, D, P, R, L.

3. The composition of claim 1, wherein X1, X3, X4, X6, and X7 are independently selected from the following groups;
X1 is selected from the group consisting of G, M, T, S, N, D;
X3 is selected from the group consisting of N, S, T, H, D;
X4 is selected from the group consisting of T, V, I, A;
X6 is selected from the group consisting of N, S, G, D, P; and
X7 is selected from T, Y, W, N, V, I, H, M, S, G, A, Q, F, D, P, R, L.

4. The composition of claim 1, wherein X1 is R or K and X3, X4, X6 and X7 are D or E.

5. The composition of claim 1, wherein
X1 is not R, K, or C;
X3 is not W, F, K, C, I, P or, L;
X4 is not Y, G, P, D, C, Q, R, K, E, F, L, or R;
X6 is not R, I, W, V, F, C, L, E, or K; or
X7 is not C, K, E.

6. The composition of claim 1, wherein the n-mer motif is selected from one of Table 1 to Table 6.

7. The composition of claim 1, wherein the n-mer motif is NNSTRGG (SEQ ID NO: 1), GNSARNI (SEQ ID NO: 2), GNSVRDF (SEQ ID NO: 3), or a combination thereof.

8. The composition of any one of the preceding claims, wherein the targeting moiety is part of a viral capsid protein.

9. The composition of claim 8, wherein the targeting motif is located between two amino acids of the viral capsid protein such that the n-mer is external to a viral capsid.

10. The composition of claim 9, wherein the viral capsid protein is an AAV viral capsid protein.

11. The composition of claim 10, wherein the targeting motif is inserted between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

12. The composition of claim 11, wherein the engineered AAV capsid protein comprises one or more mutations.

13. The composition of claim 12, wherein the one or more mutations comprise K449R of AAV9, or in an analogous position in an AAV1, AAV2, AAV3, AAV4, AAV5, AAV6, AAV7, AAV8, AAV rh.74, AAV rh.10 capsid polypeptide.

14. The composition of any one of the preceding claims, wherein the cargo is a polynucleotide, one or more polypeptides, a ribonucleoprotein complex.

15. The composition of claim 14, wherein the polynucleotide encodes one or more polypeptides and/or a RNAi oligonucleotide.

16. The composition of claim 15, wherein the polynucleotide encodes one or more polypeptides.

17. The composition of claim 16, wherein the one or more polypeptides comprise enzymes or antibodies.

18. The composition of any one of the preceding claims, wherein the polynucleotide encodes a CRISPR-Cas system.

19. The composition of claim 14, wherein the cargo is a polynucleotide, the polynucleotide comprising one or more repeat elements that reduce or eliminate expression of the polynucleotide in a non-endothelial cell of the CNS vasculature.

20. The composition of claim 19, wherein the one or more repeat elements are the hepatocyte-selective miR-122 repeat element.

21. A vector system comprising one or more vectors encoding a targeting moiety comprising an n-mer, the n-mer comprising or consisting of the targeting moiety comprising an n-mer motif, the n-mer motif comprising or consisting of X1-N-X3-X4-X5-X6-X7, wherein X5 is independently selected from K or R, and X1, X3, X4, X6 and X7 are independently selected from any amino acid, optionally wherein the overall charge of the n-mer motif at neutral pH is between 0 and +2; and a cargo polynucleotide.

22. The vector of claim 21, wherein X1, X3, X4, X6, and X7 is independently selected from the following groups;
X1 is selected from the group consisting of G, M, T, S, N, D, L, H, P, I, V, Q, Y, W, F, A, E;
X3 is selected from N, S, T, H, D, A, Y, M, Q, E, R, G, V;
X4 is selected from T, V, I, A, M, S, H, W, N;
X6 is selected from N, S, G, D, P, T, H, Q, A, Y;
X7 is selected from T, Y, W, N, V, I, H, M, S, G, A, Q, F, D, P, R, L.

23. The vector of claim 21, wherein X1, X3, X4, X6, and X7 are independently selected from the following groups;
X1 is selected from the group consisting of G, M, T, S, N, D;
X3 is selected from the group consisting of N, S, T, H, D;
X4 is selected from the group consisting of T, V, I, A;
X6 is selected from the group consisting of N, S, G, D, P; and
X7 is selected from T, Y, W, N, V, I, H, M, S, G, A, Q, F, D, P, R, L.

24. The vector of claim 21, wherein X1 is R or K and X3, X4, X6 and X7 are D or E.

25. The vector of claim 21, wherein
X1 is not R, K, or C;
X3 is not W, F, K, C, I, P or, L;
X4 is not Y, G, P, D, C, Q, R, K, E, F, L, or R;
X6 is not R, I, W, V, F, C, L, E, or K; or
X7 is not C, K, E.

26. The vector system of claim 21, wherein the n-mer is selected from any one as listed in Tables 1-6, or any combination thereof.

27. The vector system of any one of claims 21-26, wherein vector encodes the targeting moiety within a viral capsid protein.

28. The vector system of claim 27, wherein the targeting moiety is located between two amino acids of the viral capsid protein such that the n-mer is external to the viral capsid.

29. The vector system of claim 28, wherein the viral capsid protein is a AAV viral capsid protein.

30. The vector system of claim 29, wherein the n-mer motif is inserted between amino acids 588 and 589 in an AAV9 capsid polypeptide, or in an analogous position in an AAV1, AAV2, AAV3, AAV4, AAV5, AAV6, AAV7, AAV8, AAV9, AAV rh.74, AAV rh. 10 capsid polypeptide.

31. The vector system of claim 29, wherein the AAV capsid protein comprises one or more mutations.

32. The vector system of claim 31, wherein the one or more mutations comprise K449R of AAV9, or in an analogous position in AAV1, AAV2, AAV3, AAV4, AAV5, AAV6, AAV7, AAV8, AAV rh.74, AAV rh.10 capsid polypeptide.

33. The vector system of claim 21, wherein the polynucleotide encodes a RNAi oligonucleotide.

34. The vector system of claim 33, wherein the polynucleotide encodes one or more polypeptides.

35. The vector system of claim 34, wherein the polypeptides include enzymes and antibodies.

36. The vector system of any one of claims 21-35, wherein the polynucleotide encodes a CRISPR-Cas system.

37. The vector system of claim 21, wherein the cargo polynucleotide further comprise one or more repeat elements that reduce or eliminate expression of the polynucleotide in a non-endothelial cell of the CNS vasculature.

38. The vector system of claim 37, wherein the one or more repeat elements are the hepatocyte-selective miR-122 repeat element.

39. A polypeptide encoded or produced by the vector system of any one of claims 21 to 38.

40. A particle produced by the vector system of any one of claims 21 to 38.

41. A cell comprising the composition, vector, polypeptide, or particle of any of the preceding claims.

42. A method of delivering a cargo to endothelial cells of the CNS vasculature comprising;
administering, in vivo or in vitro, the composition of any one of claims 1 to 20, or the vector of any one of claims 21 to 38.

43. The method of claim 42, wherein the cargo is an RNAi oligonucleotide, a polynucleotide encoding a polypeptide, or a polypeptide.

44. The method of claim 43, wherein the polypeptide includes an enzyme or antibody.

45. The method of claim 42, wherein the cargo is a Cas polypeptide, a guide molecule, or both.
